SEQUENCE LISTING (51 sequences)

SEQ ID NO: 1 - DNA, 20 bp, Artificial Sequence
  Synthetic phosphorothioate linked oligodeoxynucleotide
cttacgctgagtacttcgat

SEQ ID NO: 2 - DNA, 37 bp, Artificial Sequence
  Exemplary G-quadruplexes sequences, synthetic oligodeoxyribonucleotide
gggnnnnnnnngggnnnnnnnngggnnnnnnnngggn

SEQ ID NO: 3 - DNA, 61 bp, Artificial Sequence
  Synthetic phosphorothioate linked oligodeoxynucleotide
ggggggggggnnnnnnnggggggggggnnnnnnnggggggggggnnnnnnngggggggggg

SEQ ID NO: 4 - protein, 29 aa, Artificial Sequence
  Exemplary endosomolytic peptide
AALEALAEALEALAEALEALAEAAAAGGC

SEQ ID NO: 5 - protein, 30 aa, Artificial Sequence
  Exemplary endosomolytic peptide
AALAEALAEALAEALAEALAEALAAAAGGC

SEQ ID NO: 6 - protein, 15 aa, Artificial Sequence
  Exemplary endosomolytic peptide
ALEALAEALEALAEA

SEQ ID NO: 7 - protein, 22 aa, Artificial Sequence
  Exemplary endosomolytic peptide
GLFEAIEGFIENGWEGMIWDYG

SEQ ID NO: 8 - protein, 23 aa, Artificial Sequence
  Exemplary endosomolytic peptide
GLFGAIAGFIENGWEGMIDGWYG

SEQ ID NO: 9 - protein, 48 aa, Artificial Sequence
  Exemplary endosomolytic peptide
GLFEAIEGFIENGWEGMIDGWYGCGLFEAIEGFIENGWEGMIDGWYGC

SEQ ID NO: 10 - protein, 44 aa, Artificial Sequence
  Exemplary endosomolytic peptide
GLFEAIEGFIENGWEGMIDGGCGLFEAIEGFIENGWEGMIDGGC

SEQ ID NO: 11 - protein, 35 aa, Artificial Sequence
  Exemplary endosomolytic peptide
GLFGALAEALAEALAEHLAEALAEALEALAAGGSC

SEQ ID NO: 12 - protein, 34 aa, Artificial Sequence
  Exemplary endosomolytic peptide
GLFEAIEGFIENGWEGLAEALAEALEALAAGGSC

SEQ ID NO: 13 - protein, 41 aa, Artificial Sequence
  Exemplary endosomolytic peptide
GLFEAIEGFIENGWEGNIDGKGLFEAIEGFIENGWEGNIDG

SEQ ID NO: 14 - protein, 16 aa, Artificial Sequence
  Exemplary cell permeation peptide
RQIKIWFQNRRMKWKK

SEQ ID NO: 15 - protein, 14 aa, Artificial Sequence
  Exemplary cell permeation peptide
GRKKRRQRRRPPQC

SEQ ID NO: 16 - protein, 27 aa, Artificial Sequence
  Exemplary cell permeation peptide
GALFLGWLGAAGSTMGAWSQPKKKRKV

SEQ ID NO: 17 - protein, 18 aa, Artificial Sequence
  Exemplary cell permeation peptide
LLIILRRRIRKQAHAHSK

SEQ ID NO: 18 - protein, 26 aa, Artificial Sequence
  Exemplary cell permeation peptide
GWTLNSAGYLLKINLKALAALAKKIL

SEQ ID NO: 19 - protein, 18 aa, Artificial Sequence
  Exemplary cell permeation peptide
KLALKLALKALKAALKLA

SEQ ID NO: 20 - protein, 9 aa, Artificial Sequence
  Exemplary cell permeation peptide
RRRRRRRRR

SEQ ID NO: 21 - protein, 10 aa, Artificial Sequence
  Exemplary cell permeation peptide
KFFKFFKFFK

SEQ ID NO: 22 - protein, 37 aa, Artificial Sequence
  Exemplary cell permeation peptide
LLGDFFRKSKEKIGKEFKRIVQRIKDFLRNLVPRTES

SEQ ID NO: 23 - protein, 31 aa, Artificial Sequence
  Exemplary cell permeation peptide
SWLSKTAKKLENSAKKRISEGIAIAIQGGPR

SEQ ID NO: 24 - protein, 30 aa, Artificial Sequence
  Exemplary cell permeation peptide
ACYCRIPACIAGERRYGTCIYQGRLWAFCC

SEQ ID NO: 25 - protein, 36 aa, Artificial Sequence
  Exemplary cell permeation peptide
DHYNCVSSGGQCLYSACPIFTKIQGTCYRGKAKCCK

SEQ ID NO: 26 - protein, 12 aa, Artificial Sequence
  Exemplary cell permeation peptide
RKCRIVVIRVCR

SEQ ID NO: 27 - protein, 42 aa, Artificial Sequence
  Exemplary cell permeation peptide
RRRPRPPYLPRPRPPPFFPPRLPPRIPPGFPPRFPPRFPGKR

SEQ ID NO: 28 - protein, 13 aa, Artificial Sequence
  Exemplary cell permeation peptide
ILPWKWPWWPWRR

SEQ ID NO: 29 - protein, 16 aa, Artificial Sequence
  Exemplary hydrophobic MTS-containing peptide, RFGF
AAVALLPAVLLALLAP

SEQ ID NO: 30 - protein, 11 aa, Artificial Sequence
  Exemplary hydrophobic MTS-containing peptide, RFGF analogue
AALLPVLLAAP

SEQ ID NO: 31 - protein, 13 aa, Artificial Sequence
  Exemplary cell membrane delivery peptide, sequences from the HIV Tat protein
GRKKRRQRRRPPQ

SEQ ID NO: 32 - protein, 16 aa, Artificial Sequence
  Exemplary cell membrane delivery peptide, the Drosophila Antennapedia protein
RQIKIWFQNRRMKWKK

SEQ ID NO: 33 - DNA, 23 bp, Mus musculus
aagctggccctggacatggagat

SEQ ID NO: 34 - DNA, 19 bp, Artificial Sequence
  Combined DNA/RNA molecule, synthetic oligonucletide
gccuggaguuuauucggaa

SEQ ID NO: 35 - DNA, 21 bp, Artificial Sequence
  Combined DNA/RNA molecule, synthetic oligonucletide
uuccgaauaaacuccaggctt

SEQ ID NO: 36 - DNA, 27 bp, Artificial Sequence
  Combined DNA/RNA molecule, synthetic oligonucletide
gccuggaguuuauucggaagagucagc

SEQ ID NO: 37 - DNA, 49 bp, Artificial Sequence
  Combined DNA/RNA molecule, synthetic oligonucletide
tggagtgtctaccactatctgcugacucuuccgaauaaacuccaggccu

SEQ ID NO: 38 - DNA, 21 bp, Artificial Sequence
  Combined DNA/RNA molecule, synthetic oligonucletide
gccuggaguuuauucggaatt

SEQ ID NO: 39 - DNA, 20 bp, Artificial Sequence
  Combined DNA/RNA molecule, synthetic oligonucletide
cuuacgcugaguacuucgau

SEQ ID NO: 40 - DNA, 19 bp, Artificial Sequence
  Combined DNA/RNA molecule, synthetic oligonucletide
uuccgaauaaacuccaggc

SEQ ID NO: 41 - DNA, 20 bp, Artificial Sequence
  Synthetic phosphorothioate linked oligodeoxynucletide
nnnnnnnnnnnnnnnnnnnn

SEQ ID NO: 42 - DNA, 29 bp, Artificial Sequence
  Combined DNA/RNA molecule, synthetic oligonucletide
cuggcugaauuucagagcagggggggggg

SEQ ID NO: 43 - DNA, 21 bp, Artificial Sequence
  Combined DNA/RNA molecule, synthetic oligonucletide
ugcucugaaauucagccagtt

SEQ ID NO: 44 - DNA, 21 bp, Artificial Sequence
  Combined DNA/RNA molecule, synthetic oligonucletide
ugcucugaaauucagccagtt

SEQ ID NO: 45 - DNA, 21 bp, Artificial Sequence
  Combined DNA/RNA molecule, synthetic oligonucletide
gccuggaguuuauucggaatt

SEQ ID NO: 46 - DNA, 20 bp, Artificial Sequence
  Combined DNA/RNA molecule, synthetic oligonucletide
cttacgctgagtacttcgat

SEQ ID NO: 47 - DNA, 21 bp, Artificial Sequence
  Combined DNA/RNA molecule, synthetic oligonucletide
ggaucaucucaagucuuactt

SEQ ID NO: 48 - DNA, 20 bp, Artificial Sequence
  Combined DNA/RNA molecule, synthetic oligonucletide
gcccaagctggcatccgtca

SEQ ID NO: 49 - DNA, 19 bp, Artificial Sequence
  Combined DNA/RNA molecule, synthetic oligonucletide
ggaucaucucaagucuuac

SEQ ID NO: 50 - DNA, 21 bp, Artificial Sequence
  Combined DNA/RNA molecule, synthetic oligonucletide
guaagacuugagaugaucctt

SEQ ID NO: 51 - DNA, 21 bp, Artificial Sequence
  Combined DNA/RNA molecule, synthetic oligonucletide
ggaucaucucaagucuuactt

(19) United States
(12) Patent Application Publication
(10) Pub. No.: US 2011/0218334 A1
(43) Pub. Date: Sep. 8, 2011

(54) PHOSPHOROTHIOATE OLIGONUCLEOTIDES AND NON-NUCLEOSIDIC PHOSPHOROTHIOATES AS DELIVERY AGENTS FOR iRNA AGENTS

(75) Inventors: Martin Maier, Cambridge, MA (US); Muthiah Manoharan, Cambridge, MA (US); Kallanthottathil G. Rajeev, Cambridge, MA (US); Kevin Fitzgerald, Cambridge, MA (US); Antonin de Fougerolles, Cambridge, MA (US); Tatiane Novobrantseva, Cambridge, MA (US); Anna Borodovsky, Cambridge, MA (US); Maria Frank-Kamenetsky, Cambridge, MA (US)

(73) Assignee: ALNYLAM PHARMACEUTICALS, INC., Cambridge, MA (US)

(21) Appl. No.: 13/003,355

(22) PCT Filed: Jul. 10, 2009
(86) PCT No.: PCT/US2009/050216

§ 371 Date: May 25, 2011

Related U.S. Application Data
(60) Provisional application No. 61/079,971, filed on Jul. 11, 2008.

(51) Int. Cl. C07H 21/02

(52) U.S. Cl. 536/24.5

(57) ABSTRACT

The invention relates to use of single-stranded phosphorothioate oligonucleotides or non-nucleosidic phosphrothiaote as vehicles or carriers to modulate the biodistribution of iRNA agents.

### PRIORITY CLAIM

This application claims priority to PCT Application No. PCT/US09/50216, filed Jul. 10, 2009, which claims priority to U.S. Provisional Application No. 61/079,971, filed Jul. 11, 2008, both of which are herein incorporated by reference in their entirety.

### GOVERNMENT FUNDING

The work described herein was supported, in part, by under government DTRA Host Factor contract HDTRA1-07-C-0082. The United States government may have certain rights in the invention.

### TECHNICAL FIELD

The invention relates to compostions and methods useful in administering nucleic acid based therapies, for example use of singe-stranded oligonucleotides or non-nucleosidic phosohorothioates as delivery vehicles.

### BACKGROUND

Many diseases (e.g., cancers, hematopoietic disorders, endocrine disorders, and immune disorders) arise from the abnormal expression or activity of a particular gene or group of genes. Similarly, disease can result through expression of a mutant form of protein, as well as from expression of viral genes that have been integrated into the genome of their host. The therapeutic benefits of being able to selectively silence these abnormal or foreign genes are obvious.

Oligonucleotide compounds have important therapeutic applications in medicine. Oligonucleotides can be used to silence genes that are responsible for a particular disease. Gene-silencing prevents formation of a protein by inhibiting translation. Importantly, gene-silencing agents are a promising alternative to traditional small, organic compounds that inhibit the function of the protein linked to the disease. siRNA, antisense oligonucleotides, and micro-RNA are oligonucleotides that prevent the formation of proteins by gene-silencing.

RNA interference or “RNAi” is a term initially coined by Fire and co-workers to describe the observation that double-stranded RNA (dsRNA) can block gene expression when it is introduced into worms (Fire et al. (1998) Nature 391, 806-811). Short dsRNA directs gene-specific, post-transcriptional silencing in many organisms, including vertebrates, and has provided a new tool for studying gene function. RNAi is mediated by RNA-induced silencing complex (RISC), a sequence-specific, multicomponent nuclease that destroys messenger RNAs homologous to the silencing trigger. RISC is known to contain short RNAs (approximately 22 nucleotides) derived from the double-stranded RNA trigger, but the protein components of this activity remained unknown.

siRNA compounds are promising agents for a variety of diagnostic and therapeutic purposes. siRNA compounds can be used to identify the function of a gene. In addition, siRNA compounds offer enormous potential as a new type of pharmaceutical agent which acts by silencing disease-causing genes. Research is currently underway to develop interference RNA therapeutic agents for the treatment of many diseases including central-nervous-system diseases, inflammatory diseases, metabolic disorders, oncology, infectious diseases, and ocular disease.

siRNAs has been shown to be extremely effective as a potential anti-viral therapeutic with numerous published examples appearing recently. siRNA molecules directed against targets in the viral genome dramatically reduce viral titers by orders of magnitude in animal models of influenza (Ge et. al., Proc. Natl. Acd. Sci. USA, 101:8676-8681 (2004); Tompkins et. al., Proc. Natl. Acd. Sci. USA, 101:8682-8686 (2004); Thomas et. al., Expert Opin. Biol. Ther. 5:495-505 (2005)), respiratory synctial virus (RSV) (Bitko et. al., Nat. Med. 11:50-55 (2005)), hepatitis B virus (HBV) (Morrissey et. al., Nat. Biotechnol. 23:1002-1007 (2005)), hepatitis C virus (Kapadia, Proc. Natl. Acad. Sci. USA, 100:2014-2018 (2003); Wilson et. al., Proc. Natl. Acad. Sci. USA, 100:2783-2788 (2003)) and SARS coronavirus (Li et. al., Nat. Med. 11:944-951 (2005)).

The most commonly used siRNAs, typically about 22 nucleotides in length; undergo rapid filtration by the kidney owing to their small size. By coupling the short dsRNAs to other molecules such as peptides, antibodies or ligands, or encasing them in liposomes or other types of particles, it is possible to produce siRNA agents that have prolonged lifetime in animals. Although, in mammalian cells, long dsRNAs can also be used as RNAi agents, the long dsRNAs induce the interferon response, which is frequently injurious to the host. One approach would be to use longer dsRNA molecules that can be easily processed by an endogenous enzyme, such as dicer, to produce short dsRNAs that do not induce interferon response and are capable inducing RNA interference. These longer dsRNA can be modified to modulate the interferon response.

Antisense methodology is the complementary hybridization of relatively short oligonucleotides to mRNA or DNA such that the normal, essential functions, such as protein synthesis, of these intracellular nucleic acids are disrupted. Hybridization is the sequence-specific hydrogen bonding via Watson-Crick base pairs of oligonucleotides to RNA or single-stranded DNA. Such base pairs are said to be complementary to one another.

The naturally-occurring events that alter the expression level of the target sequence, discussed by Cohen (Oligonucleotides: Antisense Inhibitors of Gene Expression, CRC Press, Inc., 1989, Boca Raton, Fla.) are thought to be of two types. The first, hybridization arrest, describes the terminating event in which the oligonucleotide inhibitor binds to the target nucleic acid and thus prevents, by simple steric hindrance, the binding of essential proteins, most often ribosomes, to the nucleic acid. Methyl phosphonate oligonucleotides (Miller et al. (1987) Anti-Cancer Drug Design, 2:117-128), and α-anomer oligonucleotides are the two most extensively studied antisense agents which are thought to disrupt nucleic acid function by hybridization arrest.

Another means by which antisense oligonucleotides alter the expression level of target sequences is by hybridization to a target mRNA, followed by enzymatic cleavage of the targeted RNA by intracellular RNase H. A 2′-deoxyribofuranosyl oligonucleotide or oligonucleotide analog hybridizes with the targeted RNA and this duplex activates the RNase H enzyme to cleave the RNA strand, thus destroying the normal function of the RNA. Phosphorothioate oligonucleotides are the most prominent example of an antisense agent that operates by this type of antisense terminating event.

Numerous studies have shown that oligonucleotides containing multiple phosphorothioate linkages exhibit unique pharmacokinetics and relatively broad biodistribution after systemic as well as local administration (Phillips, J. A., S. J. Craig, et al. (1997) Biochem. Pharmacol. 54, 657-68; Yu, R. Z., R. S. Geary, et al. (2001) J. Pharm. Sci. 90, 182-193; Templin, M. V., A. A. Levin, et al. (2000) Antisense Nucleic Acid Drug Dev. 10, 359-68; Yu, R. Z., H. Zhang, et al. (2001) J. Pharmacol. Exp. Ther. 296, 388-95). Although the bioavailability and biodistribution depends to some extend on the sequence and modification of the oligonucleotide, the pharmacokinetic behavior has been found to be closely correlated to the number of phosphorothioate linkages in the oligomer (Watanabe, T. A., R. S. Geary, et al. (2006) Oligonucleotides 16, 169-180). However, possibilities to modify nucleic acid therapeutics with phosphorothioate linkages are limited because while the affinity of phosphorothioates for proteins is desired for biodistribution, it could become a hindrance due to the low availability of unbound nucleic acid therapeutic.

Despite advances in siRNA, antisense and other oligonucleotide based technologies, one of the major hurdles is delivery of these molecules into cell especially in vivo delivery of these molecules. Thus there is a need in the art for novel oligonucleotide and oligonucleotide conjugate constructs that help in delivering these molecules into cells both in vitro and in vivo.

### SUMMARY OF THE INVENTION

The invention is based on the use of single-stranded or double-stranded phosphorothioate oligonucleotides or non-nucleosidic phosphorothioates as vehicles or carriers to modulate the biodistribution of iRNA agents. In one aspect, the invention provides iRNA agents linked to an oligonucleotide phosphorothioate or non-nucleosidic phosphorothioate oligomer carrier.

The invention further provides a method for delivering an iRNA agent to a cell, the method comprising the steps of contacting the iRNA agent with a cell. In certain embodiments, the cell is a human cell. In certain embodiments, the cell is non-human primate cell. In certain embodiments, the cell is a non-primate cell.

The invention further provides compositions and kits useful in practicing the methods of the invention.

The details of one or more embodiments of the invention are set forth in the accompanying drawings and the description below. Other features and advantages of the invention will be apparent from the description and drawings, and from the claims. This application incorporates all cited references, patents, and patent applications by references in their entirety for all purposes.

### BRIEF DESCRIPTION OF FIGURES

FIG. 1. Some exemplary non-nucleic acid carriers containing phosphorothioate linkages.

FIG. 2. Schematic representation of siRNA-carrier oligomer constructs designed to be a substrate for Dicer cleavage.

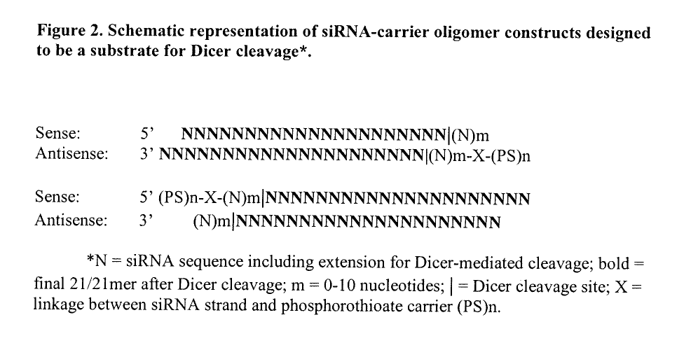

FIG. 3. Schematic of the oligonucleotide carrier approach.

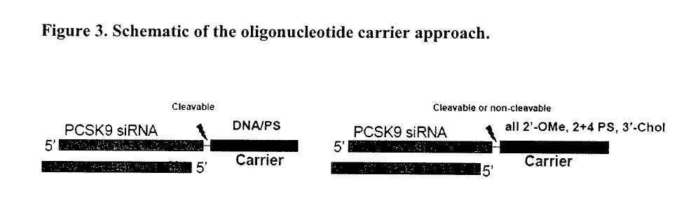

FIG. 4: Results of PCSK9 inhibition with carrier oligonucleotide conjugated siRNAs. (A) iv injection and (B) ip injection.

FIG. 5. Results of PCSK9 inhibition with carrier oligonucleotide conjugated siRNAs.

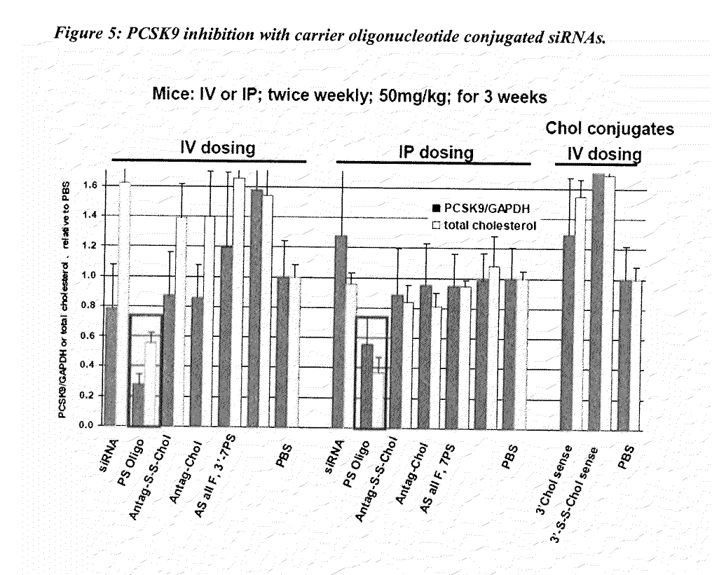

FIG. 6. Structure of the abasic (1′,2′-dideoxyribose) phosphorothioate carrier oligomer.

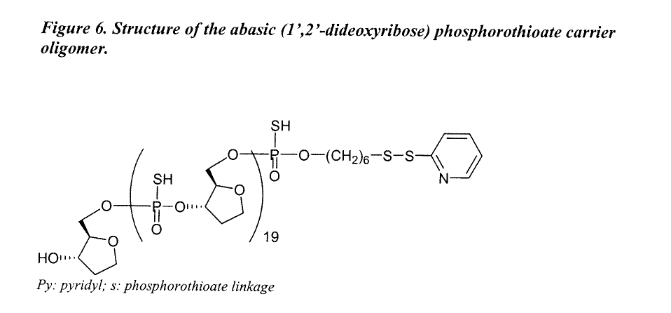

FIG. 7. INF-α responses of siRNA duplexes (A) Linear detection range 39-2500 pg/ml (B) linear detection range of 78/5000 pg/ml.

FIG. 8. Results of PCSK9 inhibition with carrier oligonucleotide conjugated siRNAs.

### DETAILED DESCRIPTION

The invention provides novel oligonucleotides and oligonucleotides conjugates for use as iRNA agents. The invention further provides methods of delivering these novel oligonucleotides and oligonucleotide conjugates to a cell.

The invention is based on the use of single-stranded or double-stranded phosphorothioate oligonucleotides or non-nucleosidic phosphorothioates as vehicles or carriers to modulate the biodistribution of iRNA agents. These oligonucleotides and the non-nucleosidic phosphorothioates are also referred to as the carrier oligomers. The oligonucleotide carrier oligomers are also referred to as carrier oligonucleotides. In one aspect, the invention provides iRNA agents linked to an oligonucleotide phosphorothioate or non-nucleosidic phosphorothioate oligomer carrier.

In one embodiment, the iRNA agent comprises at least one carrier oligomer.

In one embodiment, the iRNA agent comprises two or more carrier oligomers.

In one embodiment, the iRNA agent comprises two or more carrier oligomers that are all the same.

In one embodiment, the iRNA agent comprises two or more carrier oligomers that are all different from each other.

In one embodiment, the iRNA agent comprises three or more carrier oligomers some of which are the same and others that are different.

In one embodiment, the carrier oligomer comprises more than one phosphorothioates.

In one embodiment, the carrier oligomer is an oligonucleotide.

The length of the carrier oligonucleotide can range from 5 nucleotides to about 60 nucleotides in length. In one embodiment, the carrier oligonucleotide is between 5 to 50 nucleotides in length. In one preferred embodiment, the carrier oligonucleotide is between 5 to 40 nucleotides in length. In a more preferred embodiment, the carrier oligonucleotide is between 5 to 35 nucleotides in length. In an even more preferred embodiment, the carrier oligonucleotide is between 5 and 30 nucleotides in length.

In one embodiment, the carrier oligonucleotide is between 5 and 25 nucleotides in length. In a preferred embodiment the carrier oligonucleotide is 20 nucleotides in length.

In one embodiment, at least 10%, at least 20%, at least 30%, at least 40%, at least 50%, at least 60%, at least 70%, at least 80%, at least 90% or 100% of the backbone linkages in the carrier oligonucleotide are phosphorothioate linkages.

In one preferred embodiment, the carrier oligonucleotide has the sequence dCsdTsdTdsAsdCsdGsdCsdTsdGsdAsdGsdTsdAsdCsdTsdTsdCsdGsdAsdT (SEQ ID NO: 1).

In one embodiment, the carrier oligonucleotide comprises at least one backbone modifications in addition to the phosphorothioate, e.g. phosphorodithioate, methylphosphonate, phosphoramidate backbone linkage.

In one embodiment, the carrier oligonucleotide comprises at least one phosphorodithioate linkages.

In one embodiment, the carrier oligonucleotide comprises at least one methylthiophosphonate linkage.

In one embodiment, the carrier oligonucleotide comprises at least one phosphoramidate backbone linkage.

The scavenger receptors (SRs) are pattern recognition receptors with a common specificity of binding to polyanionic ligands. Class A SRs are homotrimeric type II transmembrane glycoproteins with an N-terminal cytoplasmic domain, an α-helical coiled domain, collagenous domain, and a C-terminal scavenger receptor cysteine-rich domain. Poly G oligonucleotides have been shown to be recognized by macrophage scavenger receptors, such as SR-A and CD36. Harshyne et al. have shown that poly(G) oligonucleotides and polyguanylic acid bind to SRs on dendritic cells. Prasad et al. have shown that a 10-mer poly(G) oligonucleotides binds to SRs on macrophages. Peasron et al. have demonstrated that poly(G) and poly(I) (I=inosine), but not poly(C) and poly(A), interact with SR-A in CHO cells. Pearson et al. attributed this difference in the ability of the poly(G) and poly(I) to form quadraplexes. Thus in one aspect the invention provides carrier oligomer linked iRNA agents which are preferentially delivered to macrophages and dendritic cells.

In one embodiment, the carrier is a poly(G) oligonucleotide. In a preferred embodiment, the carrier poly(G) oligonucleotide is 3-14 nucleotides, more preferably 5-10 nucleotides in length. In an even more preferred embodiment, the carrier poly(G) oligonucleotide is d(G)₅. In one preferred embodiment, the carrier poly(G) oligonucleotide is d(G)₁₀.

In one embodiment, the carrier is a poly(I) oligonucleotide.

In one embodiment, the carrier is a poly(C) oligonucleotide.

In one embodiment, the carrier is a poly(A) oligonucleotide.

In one embodiment, the carrier is a poly(U) oligonucleotide.

In one embodiment, the carrier is a poly(dT) oligonucleotide.

Certain sequences are known to form four stranded structures which are referred to as quadruplexes or tetraplexes. Many examples of sequences that form G-quadruplexes are known in the art, e.g. Todd et al Nucleic Acids Research (2005) 33(9):2901-2907 and Burge et al Nucleic Acids Research (2006) 34(19):5402-5415. A rule for predicting the formation of G-quadruplexes has been proposed, where sequences are predicted to fold based on the pattern d(G₃₊N₁₋₇G₃₊N₁₋₇G₃₊N₁₋₇G₃₊) (SEQ ID NO: 2), where N is any nucleobase (including guanine) (Huppert et al Nucleic Acids Rersearc (2005) 33(9): 2908-2916) This rule has been widely used in on-line algorithms to predict sequences that can form G-quadruplexes.

In one embodiment, the carrier is an oligonucleotide that forms a quadruplex structure, for example a G-quartet.

In one embodiment, the carrier oligonucleotide has the sequence d(GₚN_{q}GₚN_{q}GₚN_{q}Gₚ) (SEQ ID NO: 3); wherein:

p is independently for each occurrence 3-10;

q is independently for each occurrence 1-7; and

N is independently for each occurrence an optionally modified natural or non-natural nucleobase.

In one embodiment, at least one N is not guanine.

In one embodiment, the carrier oligonucleotide comprises at least one optionally substituted modified or non-natural nucleobase, for example optionally substituted difluorortoluoyl (DFT) or optionally substituted nitroindole. Copending U.S. application Ser. No. 11/186,915, filed Jul. 21, 2005, describes additional modified or non-natural nucleobases that are amenable to modifying the carrier oligonucleotide.

In one embodiment, the carrier oligonucleotide modulates the immune response, for example the oligonucleotide comprises a 5′-CpG-3′ dinucleotide.

In one embodiment, the carrier oligonucleotide comprises no 5′-CpG-3′ dinucleotides in the sequence.

In one embodiment, the carrier oligonucleotide comprises 5-methyl-cytosine in place of cytosine.

In one embodiment, the carrier oligonucleotide comprises 5-methyl-cytosine in place of cytosine in the 5′-CpG-3′ dinucleotides in the sequence.

In one embodiment, the carrier oligonucleotide comprises an optionally substituted modified or non-natural nucleotide in place of cytosine in the 5′-CpG-3′ dinucleotide in the sequence.

In one embodiment, the carrier oligonucleotide comprises sugar modifications, for example modification at the 2′-position of the ribose sugar, e.g. 2′-fluoro, 2′-O-methyl, 2′-methoxyethyl (2′-MOE). In one embodiment, the carrier oligonucleotide comprises at least one 2′-arabinose sugar. In one embodiment, the carrier oligonucleotide comprises at least one 2′-arabinose sugar wherein the 2′-OH has been replaced (e.g. 2′-arabinose-2′-fluoro) or is modified with an alkyl (e.g. 2′-arabinose-2′-O-methyl).

In one embodiment, the carrier oligonucleotide comprises a 2′-modified ribose sugar at the terminal end.

In one embodiment, the carrier oligonucleotide comprises 2′-modified ribose sugar at the terminal end which is not linked to the iRNA agent.

In one embodiment, the carrier oligonucleotide comprises at least one modification that enhances serum stability of the carrier oligonucleotide.

In one embodiment, the carrier oligonucleotide is fully DNA.

In one embodiment, the carrier oligonucleotide is fully RNA.

In one embodiment, the carrier oligonucleotide is a mix of DNA and RNA.

In one embodiment, the carrier oligonucleotide is double-stranded. The double-stranded oligonucleotide carrier can range in length from 5 to 50 base pairs. In a preferred embodiment, the double-stranded oligonucleotide carrier is 5 to 40 base pairs long. In an even more preferred embodiment, the double-stranded oligonucleotide carrier is 5 to 30 base pairs long. In a most preferred embodiment, the double-stranded oligonucleotide carrier is 5 to 20 base pairs long.

In one embodiment, at least one strand of the double-stranded oligonucleotide carrier comprises at least one modification described herein, for example a backbone modification, a nucleobase modification, a ribose sugar modification, a terminal modification or terminal overhangs.

In one embodiment, the two strands of a double-stranded carrier oligonucleotide are covalently linked together. The covalent linkage can be nucleotidyl (for example based on naturally occurring nucleotides such as A, G, C, T or U) or non-nucleotidyl (for example based on an alkyl linker such as C₄ or C₆ alkyl)

In one embodiment, the iRNA agent is processed by Dicer before loading into the RISC.

In one embodiment, the carrier oligomer is covalently linked to the iRNA agent directly, for example through a phosphate, phosphorothioate, phosphorodithioate, alkylphosphonate (e.g. methylphosphonate), amide, ester, disulfide, thioether, oxime and hydrazone linkage. When the carrier oligomer is linked directly to the iRNA agent the linking group can be a cleavable linking group. In one embodiment, the linking group between the carrier oligomer and the iRNA agent is a cleavable linking group.

In one embodiment, the carrier oligonucleotide is linked to the iRNA agent through a spacer. The spacer can preferably be selected from the group consisting of optionally substituted alkyl, alkenyl, alkynyl, cycloalkyl, heterocyclyl or heteroaryl.

In one embodiment, the spacer comprises a cleavable linking group, for example an group that is potentially biodegradable by enzymes present in the organism such as nucleases and proteases or cleavable at acidic pH or under reductive conditions, such as by glutathione present at high levels intracelullarly. Some exemplary cleavable linking groups include, but are not limited to, disulfides, amides, esters, peptide linkages and phosphodiesters. Copending U.S. application Ser. No. 10/985,426, filed Nov. 9, 2004, describes cleavable tethers that are amenable for use as spacers comprising cleavable groups.

The cleavable linking group can be internal to the spacer or may be present at one or both terminal ends of the spacer. In one embodiment, the cleavable linking group is between the carrier oligomer and the spacer. In one embodiment, the cleavable linking group is between the carrier oligomer and the iRNA agent. In one embodiment, the cleavable linking group is internal to the spacer.

In one embodiment, the spacer is —P(Z¹)(Z²)—O—(CH₂)ₙ¹—S—S—(CH₂)ₙ²—O—P(Z³)(Z⁴)—; wherein Z¹ and Z³ are independently O or S; Z² and Z⁴ are independently O, S, alkyl (e.g. methyl) or aminoalkyl; n¹ and n² are each independently 1-10. In one preferred embodiment, n¹ and n² each are 6. In one preferred embodiment, Z¹ and Z³ each are S and Z² and Z⁴ each are O. In an even more preferred embodiment, n¹ and n² each are 6; Z¹ and Z³ each are S; Z² and Z⁴ each are O.

In one embodiment, the carrier oligonucleotide is biologically inactive, i.e. it has no known biological function other than binding to a serum protein.

In one embodiment, the carrier oligonucleotide does not target any endogenous genes.

In one embodiment, the carrier oligomer does not target any endogenous nucleic acid sequence through sequence-specific Watson-Crick base pairings.

In one embodiment, the carrier oligonucleotide inhibits the expression of a target gene.

In one embodiment, the carrier oligomer targets, through sequence-specific Watson-Crick base pairings, an endogenous gene that is different from the target of the iRNA agent.

In one embodiment, the carrier oligomer targets, through sequence-specific Watson-Crick base pairings, an endogenous gene that is the target of the iRNA agent.

In one embodiment, the carrier oligonucleotide inhibits the function of a microRNA.

In one embodiment, the carrier oligonucleotide binds to a receptor through a sequence specific motif.

In one embodiment, the carrier oligonucleotide is linked to the 5′-end of the iRNA agent.

In one embodiment, the carrier oligonucleotide is linked to the 3′-end of the iRNA agent.

In one embodiment, the carrier oligonucleotide is linked to the 5′-end of the sense strand of a double-stranded iRNA agent.

In one embodiment, the carrier oligonucleotide is linked to the 3′-end of the sense strand of a double-stranded iRNA agent.

In one embodiment, the carrier oligonucleotide is linked to the 5′-end of the antisense strand of a double-stranded iRNA agent.

In one embodiment, the carrier oligonucleotide is linked to the 3′-end of the antisense strand of a double-stranded iRNA agent.

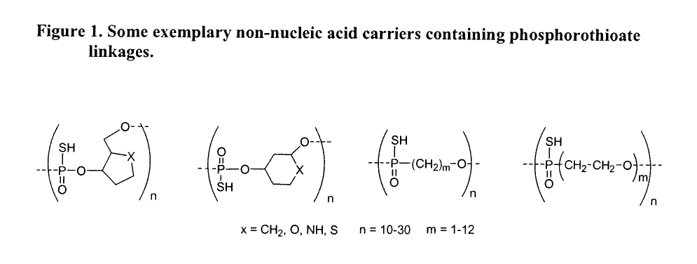

A non-nucleosidic oligomer comprises phosphorothioate functionalities tethered into an oligomeric structure through various spacers. The spacers can be, but are not limited to, abasic sugars, open chain spacers based on alkyl, alkenyl, alkynyl or ethylenoxide units or ring structures based on alkyl or ethylenoxide units, examples of some non-nucleosidic oligomers are shown in FIG. 1. The spacers can also be based on heterocyclyl and heteroaryls. The open chain spacers may be optionally inserted with N, O, or S.

In one embodiment, the carrier oligomer is a non-nucleosidic oligomer, for example the non-nucleosidic oligomers shown in FIG. 1.

Both the oligonucleotide carrier and the non-nucleosidic oligomer carrier can be synthesized separately from iRNA agent by methods known in the art and then linked with the iRNA agent post synthetically. On the other hand, the carrier oligomer (oligonucleotide or the non-nucleosidic oligomer) can be synthesized by extending the iRNA agent using the appropriate monomers.

### Ligands

A wide variety of entities can be coupled to the iRNA agent and/or the oligomer carrier. Preferred entities can be coupled to the iRNA agent and/or the oligomer carrier at various places, for example, 3′-end, 5′-end, and/or at an internal position.

Preferred moieties are ligands, which are coupled, preferably covalently, either directly or indirectly via an intervening tether. In preferred embodiments, the ligand is attached to the iRNA agent or the carrier oligomer via an intervening tether. The ligand or tethered ligand may be present on a monomer when said monomer is incorporated into the growing strand. In some embodiments, the ligand may be incorporated via coupling to a “precursor” monomer after said “precursor” monomer has been incorporated into the growing strand. For example, a monomer having, e.g., an amino-terminated tether (i.e., having no associated ligand), e.g., TAP-(CH₂)ₙNH₂ may be incorporated into a growing sense or antisense strand. In a subsequent operation, i.e., after incorporation of the precursor monomer into the strand, a ligand having an electrophilic group, e.g., a pentafluorophenyl ester or aldehyde group, can subsequently be attached to the precursor monomer by coupling the electrophilic group of the ligand with the terminal nucleophilic group of the precursor monomer's tether.

In preferred embodiments, a ligand alters the distribution, targeting or lifetime of an iRNA agent into which it is incorporated. In preferred embodiments a ligand provides an enhanced affinity for a selected target, e.g., molecule, cell or cell type, compartment, e.g., a cellular or organ compartment, tissue, organ or region of the body, as, e.g., compared to a species absent such a ligand. Preferred ligands will not take part in duplex pairing in a duplexed nucleic acid.

Preferred ligands can have endosomolytic properties. The endosomolytic ligands promote the lysis of the endosome and/or transport of the composition of the invention, or its components, from the endosome to the cytoplasm of the cell. The endosomolytic ligand may be a polyanionic peptide or peptidomimetic which shows pH-dependent membrane activity and fusogenicity. In certain embodiments, the endosomolytic ligand assumes its active conformation at endosomal pH. The “active” conformation is that conformation in which the endosomolytic ligand promotes lysis of the endosome and/or transport of the composition of the invention, or its components, from the endosome to the cytoplasm of the cell. Exemplary endosomolytic ligands include the GALA peptide (Subbarao et al., Biochemistry, 1987, 26: 2964-2972), the EALA peptide (Vogel et al., J. Am. Chem. Soc., 1996, 118: 1581-1586), and their derivatives (Turk et al., Biochem. Biophys. Acta, 2002, 1559: 56-68). In certain embodiments, the endosomolytic component may contain a chemical group (e.g., an amino acid) which will undergo a change in charge or protonation in response to a change in pH. The endosomolytic component may be linear or branched. Exemplary primary sequences of peptide based endosomolytic ligands are shown in table 1.

TABLE 1
List of peptides with endosomolytic activity.

| Name | Sequence (N to C) | Ref. |
|---|---|---|
| GALA | AALEALAEALEALAEALEALAEAAAAGGC | 1 |
| | (SEQ ID NO: 4) | |
| EALA | AALAEALAEALAEALAEALAEALAAAAGGC | 2 |
| | (SEQ ID NO: 5) | |
| | ALEALAEALEALAEA | 3 |
| | (SEQ ID NO: 6) | |
| INF-7 | GLFEAIEGFIENGWEGMIWDYG | 4 |
| | (SEQ ID NO: 7) | |
| Inf HA-2 | GLFGAIAGFIENGWEGMIDGWYG | 5 |
| | (SEQ ID NO: 8) | |
| diINF-7 | GLF EAI EGFI ENGW EGMI DGWYGC | 5 |
| | GLF EAI EGFI ENGW EGMI DGWYGC | |
| | (SEQ ID NO: 9) | |
| diINF3 | GLF EAI EGFI ENGW EGMI DGGC | 6 |
| | GLF EAI EGFI ENGW EGMI DGGC | |
| | (SEQ ID NO: 10) | |
| GLF | GLFGALAEALAEALAEHLAEALAEALEALAAGGSC | 6 |
| | (SEQ ID NO: 11) | |
| GALA-INF3 | GLFEAIEGFIENGWEGLAEALAEALEALAAGGSC | |
| | (SEQ ID NO: 12) | |
| INF-5 | GLF EAI EGFI ENGW EGnI DG K | 4 |
| | GLF EAI EGFI ENGW EGnI DG | |
| | (SEQ ID NO: 13) | |

n, norleucine
References
1. Subbarao et al., Biochemistry, 1987, 26: 2964-2972.
2. Vogel et al., J. Am. Chem. Soc., 1996, 118: 1581-1586
3. Turk, M. J., Reddy, J. A. et al. (2002). Characterization of a novel pH-sensitive peptide that enhances drug release from folate-targeted liposomes at endosomal pHs. Biochim. Biophys. Acta 1559, 56-68.
4. Plank, C. Oberhauser, B. Mechtler, K. Koch, C. Wagner, E. (1994). The influence of endosome-disruptive peptides on gene transfer using synthetic virus-like gene transfer systems, J. Biol. Chem. 269 12918-12924.
5. Mastrobattista, E., Koning, G. A. et al. (2002). Functional characterization of an endosome-disruptive peptide and its application in cytosolic delivery of immunoliposome-entrapped proteins. J. Biol. Chem. 277, 27135-43.
6. Oberhauser, B., Plank, C. et al. (1995). Enhancing endosomal exit of nucleic acids using pH-sensitive viral fusion peptides. Deliv. Strategies Antisense Oligonucleotide Ther. 247-66.

Preferred ligands can improve transport, hybridization, and specificity properties and may also improve nuclease resistance of the resultant natural or modified oligoribonucleotide, or a polymeric molecule comprising any combination of monomers described herein and/or natural or modified ribonucleotides.

Ligands in general can include therapeutic modifiers, e.g., for enhancing uptake; diagnostic compounds or reporter groups e.g., for monitoring distribution; cross-linking agents; and nuclease-resistance conferring moieties. General examples include lipids, steroids, vitamins, sugars, proteins, peptides, polyamines, and peptide mimics.

Ligands can include a naturally occurring substance, such as a protein (e.g., human serum albumin (HSA), low-density lipoprotein (LDL), high-density lipoprotein (HDL), or globulin); an carbohydrate (e.g., a dextran, pullulan, chitin, chitosan, inulin, cyclodextrin or hyaluronic acid); or a lipid. The ligand may also be a recombinant or synthetic molecule, such as a synthetic polymer, e.g., a synthetic polyamino acid, an oligonucleotide (e.g. an aptamer). Examples of polyamino acids include polyamino acid is a polylysine (PLL), poly L-aspartic acid, poly L-glutamic acid, styrene-maleic acid anhydride copolymer, poly(L-lactide-co-glycolied) copolymer, divinyl ether-maleic anhydride copolymer, N-(2-hydroxypropyl)methacrylamide copolymer (HMPA), polyethylene glycol (PEG), polyvinyl alcohol (PVA), polyurethane, poly(2-ethylacryllic acid), N-isopropylacrylamide polymers, or polyphosphazine. Example of polyamines include: polyethylenimine, polylysine (PLL), spermine, spermidine, polyamine, pseudopeptide-polyamine, peptidomimetic polyamine, dendrimer polyamine, arginine, amidine, protamine, cationic lipid, cationic porphyrin, quaternary salt of a polyamine, or an alpha helical peptide.

Ligands can also include targeting groups, e.g., a cell or tissue targeting agent, e.g., a lectin, glycoprotein, lipid or protein, e.g., an antibody, that binds to a specified cell type such as a kidney cell. A targeting group can be a thyrotropin, melanotropin, lectin, glycoprotein, surfactant protein A, Mucin carbohydrate, multivalent lactose, multivalent galactose, N-acetyl-galactosamine, N-acetyl-gulucosamine multivalent mannose, multivalent fucose, glycosylated polyaminoacids, multivalent galactose, transferrin, bisphosphonate, polyglutamate, polyaspartate, a lipid, cholesterol, a steroid, bile acid, folate, vitamin B12, biotin, an RGD peptide, an RGD peptide mimetic or an aptamer. Table 2 shows some examples of targeting ligands and their associated receptors.

TABLE 2
Targeting Ligands and their associated receptors

| Liver Cells | Ligand | Receptor |
|---|---|---|
| 1) Parenchymal | Galactose | ASGP-R |
| Cell (PC) | | (Asiologlycoprotein receptor) |
| (Hepatocytes) | Gal NAc | ASPG-R |
| | (n-acetyl-galactosamine) | Gal NAc Receptor |
| | Lactose | |
| | Asialofetuin | ASPG-r |
| 2) Sinusoidal | Hyaluronan | Hyaluronan receptor |
| Endothelial | Procollagen | Procollagen receptor |
| Cell (SEC) | Negatively charged | Scavenger receptors |
| | molecules | |
| | Mannose | Mannose receptors |
| | N-acetyl Glucosamine | Scavenger receptors |
| | Immunoglobulins | Fc Receptor |
| | LPS | CD14 Receptor |
| | Insulin | Receptor mediated transcytosis |
| | Transferrin | Receptor mediated transcytosis |
| | Albumins | Non-specific |
| | Sugar-Albumin conjugates | |
| | Mannose-6-phosphate | Mannose-6-phosphate receptor |
| 3) Kupffer | Mannose | Mannose receptors |
| Cell (KC) | Fucose | Fucose receptors |
| | Albumins | Non-specific |
| | Mannose-albumin | |
| | conjugates | |

Other examples of ligands include dyes, intercalating agents (e.g. acridines), cross-linkers (e.g. psoralene, mitomycin C), porphyrins (TPPC4, texaphyrin, Sapphyrin), polycyclic aromatic hydrocarbons (e.g., phenazine, dihydrophenazine), artificial endonucleases (e.g. EDTA), lipophilic molecules, e.g, cholesterol, cholic acid, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid, O3-(oleoyl)lithocholic acid, O3-(oleoyl)cholenic acid, dimethoxytrityl, or phenoxazine) and peptide conjugates (e.g., antennapedia peptide, Tat peptide), alkylating agents, phosphate, amino, mercapto, PEG (e.g., PEG-40K), MPEG, [MPEG]₂, polyamino, alkyl, substituted alkyl, radiolabeled markers, enzymes, haptens (e.g. biotin), transport/absorption facilitators (e.g., aspirin, vitamin E, folic acid), synthetic ribonucleases (e.g., imidazole, bisimidazole, histamine, imidazole clusters, acridine-imidazole conjugates, Eu3+ complexes of tetraazamacrocycles), dinitrophenyl, HRP, or AP.

Ligands can be proteins, e.g., glycoproteins, or peptides, e.g., molecules having a specific affinity for a co-ligand, or antibodies e.g., an antibody, that binds to a specified cell type such as a cancer cell, endothelial cell, or bone cell. Ligands may also include hormones and hormone receptors. They can also include non-peptidic species, such as lipids, lectins, carbohydrates, vitamins, cofactors, multivalent lactose, multivalent galactose, N-acetyl-galactosamine, N-acetyl-gulucosamine multivalent mannose, multivalent fucose, or aptamers. The ligand can be, for example, a lipopolysaccharide, an activator of p38 MAP kinase, or an activator of NF-κB.

The ligand can be a substance, e.g, a drug, which can increase the uptake of the iRNA agent into the cell, for example, by disrupting the cell's cytoskeleton, e.g., by disrupting the cell's microtubules, microfilaments, and/or intermediate filaments. The drug can be, for example, taxon, vincristine, vinblastine, cytochalasin, nocodazole, japlakinolide, latrunculin A, phalloidin, swinholide A, indanocine, or myoservin.

The ligand can increase the uptake of the iRNA agent into the cell by activating an inflammatory response, for example. Exemplary ligands that would have such an effect include tumor necrosis factor alpha (TNFalpha), interleukin-1 beta, or gamma interferon.

In one aspect, the ligand is a lipid or lipid-based molecule. Such a lipid or lipid-based molecule preferably binds a serum protein, e.g., human serum albumin (HSA). An HSA binding ligand allows for distribution of the conjugate to a target tissue, e.g., a non-kidney target tissue of the body. For example, the target tissue can be the liver, including parenchymal cells of the liver. Other molecules that can bind HSA can also be used as ligands. For example, neproxin or aspirin can be used. A lipid or lipid-based ligand can (a) increase resistance to degradation of the conjugate, (b) increase targeting or transport into a target cell or cell membrane, and/or (c) can be used to adjust binding to a serum protein, e.g., HSA.

A lipid based ligand can be used to modulate, e.g., control the binding of the conjugate to a target tissue. For example, a lipid or lipid-based ligand that binds to HSA more strongly will be less likely to be targeted to the kidney and therefore less likely to be cleared from the body. A lipid or lipid-based ligand that binds to HSA less strongly can be used to target the conjugate to the kidney.

In a preferred embodiment, the lipid based ligand binds HSA. Preferably, it binds HSA with a sufficient affinity such that the conjugate will be preferably distributed to a non-kidney tissue. However, it is preferred that the affinity not be so strong that the HSA-ligand binding cannot be reversed.

In another preferred embodiment, the lipid based ligand binds HSA weakly or not at all, such that the conjugate will be preferably distributed to the kidney. Other moieties that target to kidney cells can also be used in place of or in addition to the lipid based ligand.

In another aspect, the ligand is a moiety, e.g., a vitamin, which is taken up by a target cell, e.g., a proliferating cell. These are particularly useful for treating disorders characterized by unwanted cell proliferation, e.g., of the malignant or non-malignant type, e.g., cancer cells. Exemplary vitamins include vitamin A, E, and K. Other exemplary vitamins include are B vitamin, e.g., folic acid, B12, riboflavin, biotin, pyridoxal or other vitamins or nutrients taken up by cancer cells. Also included are HAS, low density lipoprotein (LDL) and high-density lipoprotein (HDL).

In another aspect, the ligand is a cell-permeation agent, preferably a helical cell-permeation agent. Preferably, the agent is amphipathic. An exemplary agent is a peptide such as tat or antennopedia. If the agent is a peptide, it can be modified, including a peptidylmimetic, invertomers, non-peptide or pseudo-peptide linkages, and use of D-amino acids. The helical agent is preferably an alpha-helical agent, which preferably has a lipophilic and a lipophobic phase.

The ligand can be a peptide or peptidomimetic. A peptidomimetic (also referred to herein as an oligopeptidomimetic) is a molecule capable of folding into a defined three-dimensional structure similar to a natural peptide. The attachment of peptide and peptidomimetics to iRNA agents can affect pharmacokinetic distribution of the iRNA, such as by enhancing cellular recognition and absorption. The peptide or peptidomimetic moiety can be about 5-50 amino acids long, e.g., about 5, 10, 15, 20, 25, 30, 35, 40, 45, or 50 amino acids long (see Table 3, for example).

TABLE 3
Exemplary Cell Permeation Peptides

| Cell Permeation | | |
|---|---|---|
| Peptide | Amino acid Sequence | Reference |
| Penetratin | RQIKIWFQNRRMKWKK (SEQ ID NO: 14) | Derossi et al., J. Biol. |
| | | Chem. 269: 10444, 1994 |
| Tat fragment | GRKKRRQRRRPPQC (SEQ ID NO: 15) | Vives et al., J. Biol. |
| (48-60) | | Chem., 272: 16010, 1997 |
| Signal Sequence- | GALFLGWLGAAGSTMGAWSQPKKKRKV | Chaloin et al., Biochem. |
| based peptide | (SEQ ID NO: 16) | Biophys. Res. Commun., |
| | | 243: 601, 1998 |
| PVEC | LLIILRRRIRKQAHAHSK(SEQ ID NO: 17) | Elmquist et al., Exp. |
| | | Cell Res., 269: 237, 2001 |
| Transportan | GWTLNSAGYLLKINLKALAALAKKIL | Pooga et al., FASEB J., |
| | (SEQ ID NO: 18) | 12:67, 1998 |
| Amphiphilic | KLALKLALKALKAALKLA (SEQ ID NO: 19) | Oehlke et al., Mol. |
| model peptide | | Ther., 2: 339, 2000 |
| Arg₉ | RRRRRRRRR (SEQ ID NO: 20) | Mitchell et al., J. Pept. |
| | | Res., 56: 318, 2000 |
| Bacterial cell | KFFKFFKFFK (SEQ ID NO: 21) | |
| wall permeating | | |
| LL-37 | LLGDFFRKSKEKIGKEFKRIVQRIKDFLRNLVPRTES | |
| | (SEQ ID NO: 22) | |
| Cecropin PI | SWLSKTAKKLENSAKKRISEGIAIAIQGGPR | |
| | (SEQ ID NO: 23) | |
| α-defensin | ACYCRIPACIAGERRYGTCIYQGRLWAFCC | |
| | (SEQ ID NO: 24) | |
| b-defensin | DHYNCVSSGGQCLYSACPIFTKIQGTCYRGKAKCCK | |
| | (SEQ ID NO: 25) | |
| Bactenecin | RKCRIVVIRVCR (SEQ ID NO: 26) | |
| PR-39 | RRRPRPPYLPRPRPPPFFPPRLPPRIPPGFPPRFPPRFPG | |
| | KR-NH2 (SEQ ID NO: 27) | |
| Indolicidin | ILPWKWPWWPWRR-NH2 (SEQ ID NO: 28) | |

A peptide or peptidomimetic can be, for example, a cell permeation peptide, cationic peptide, amphipathic peptide, or hydrophobic peptide (e.g., consisting primarily of Tyr, Trp or Phe). The peptide moiety can be a dendrimer peptide, constrained peptide or crosslinked peptide. In another alternative, the peptide moiety can include a hydrophobic membrane translocation sequence (MTS). An exemplary hydrophobic MTS-containing peptide is RFGF having the amino acid sequence AAVALLPAVLLALLAP (SEQ ID NO: 29). An RFGF analogue (e.g., amino acid sequence AALLPVLLAAP (SEQ ID NO: 30)) containing a hydrophobic MTS can also be a targeting moiety. The peptide moiety can be a “delivery” peptide, which can carry large polar molecules including peptides, oligonucleotides, and protein across cell membranes. For example, sequences from the HIV Tat protein (GRKKRRQRRRPPQ (SEQ ID NO: 31)) and the Drosophila Antennapedia protein (RQIKIWFQNRRMKWKK (SEQ ID NO: 32)) have been found to be capable of functioning as delivery peptides. A peptide or peptidomimetic can be encoded by a random sequence of DNA, such as a peptide identified from a phage-display library, or one-bead-one-compound (OBOC) combinatorial library (Lam et al., Nature, 354:82-84, 1991). Preferably the peptide or peptidomimetic tethered to an iRNA agent via an incorporated monomer unit is a cell targeting peptide such as an arginine-glycine-aspartic acid (RGD)-peptide, or RGD mimic. A peptide moiety can range in length from about 5 amino acids to about 40 amino acids. The peptide moieties can have a structural modification, such as to increase stability or direct conformational properties. Any of the structural modifications described below can be utilized.

An RGD peptide moiety can be used to target a tumor cell, such as an endothelial tumor cell or a breast cancer tumor cell (Zitzmann et al., Cancer Res., 62:5139-43, 2002). An RGD peptide can facilitate targeting of an iRNA agent to tumors of a variety of other tissues, including the lung, kidney, spleen, or liver (Aoki et al., Cancer Gene Therapy 8:783-787, 2001). Preferably, the RGD peptide will facilitate targeting of an iRNA agent to the kidney. The RGD peptide can be linear or cyclic, and can be modified, e.g., glycosylated or methylated to facilitate targeting to specific tissues. For example, a glycosylated RGD peptide can deliver an iRNA agent to a tumor cell expressing α_{V}β₃ (Haubner et al., Jour. Nucl. Med., 42:326-336, 2001).

Peptides that target markers enriched in proliferating cells can be used. E.g., RGD containing peptides and peptidomimetics can target cancer cells, in particular cells that exhibit an Iᵥθ₃ integrin. Thus, one could use RGD peptides, cyclic peptides containing RGD, RGD peptides that include D-amino acids, as well as synthetic RGD mimics. In addition to RGD, one can use other moieties that target the Iᵥ-θ₃ integrin ligand. Generally, such ligands can be used to control proliferating cells and angiogenesis. Preferred conjugates of this type ligands that targets PECAM-1, VEGF, or other cancer gene, e.g., a cancer gene described herein.

A “cell permeation peptide” is capable of permeating a cell, e.g., a microbial cell, such as a bacterial or fungal cell, or a mammalian cell, such as a human cell. A microbial cell-permeating peptide can be, for example, an α-helical linear peptide (e.g., LL-37 or Ceropin P1), a disulfide bond-containing peptide (e.g., α-defensin, β-defensin or bactenecin), or a peptide containing only one or two dominating amino acids (e.g., PR-39 or indolicidin). A cell permeation peptide can also include a nuclear localization signal (NLS). For example, a cell permeation peptide can be a bipartite amphipathic peptide, such as MPG, which is derived from the fusion peptide domain of HIV-1 gp41 and the NLS of SV40 large T antigen (Simeoni et al., Nucl. Acids Res. 31:2717-2724, 2003).

In one embodiment, a targeting peptide tethered to an iRNA agent and/or the carrier oligomer can be an amphipathic α-helical peptide. Exemplary amphipathic α-helical peptides include, but are not limited to, cecropins, lycotoxins, paradaxins, buforin, CPF, bombinin-like peptide (BLP), cathelicidins, ceratotoxins, S. clava peptides, hagfish intestinal antimicrobial peptides (HFIAPs), magainines, brevinins-2, dermaseptins, melittins, pleurocidin, H₂A peptides, Xenopus peptides, esculentinis-1, and caerins. A number of factors will preferably be considered to maintain the integrity of helix stability. For example, a maximum number of helix stabilization residues will be utilized (e.g., leu, ala, or lys), and a minimum number helix destabilization residues will be utilized (e.g., proline, or cyclic monomeric units. The capping residue will be considered (for example Gly is an exemplary N-capping residue and/or C-terminal amidation can be used to provide an extra H-bond to stabilize the helix. Formation of salt bridges between residues with opposite charges, separated by i±3, or i±4 positions can provide stability. For example, cationic residues such as lysine, arginine, homo-arginine, ornithine or histidine can form salt bridges with the anionic residues glutamate or aspartate.

Peptide and peptidomimetic ligands include those having naturally occurring or modified peptides, e.g., D or L peptides; α, β, or γ peptides; N-methyl peptides; azapeptides; peptides having one or more amide, i.e., peptide, linkages replaced with one or more urea, thiourea, carbamate, or sulfonyl urea linkages; or cyclic peptides.

The targeting ligand can be any ligand that is capable of targeting a specific receptor. Examples are: folate, GalNAc, galactose, mannose, mannose-6P, clusters of sugars such as GalNAc cluster, mannose cluster, galactose cluster, or an apatamer. A cluster is a combination of two or more sugar units. The targeting ligands also include integrin receptor ligands, Chemokine receptor ligands, transferrin, biotin, serotonin receptor ligands, PSMA, endothelin, GCPII, somatostatin, LDL and HDL ligands. The ligands can also be based on nucleic acid, e.g., an aptamer. The aptamer can be unmodified or have any combination of modifications disclosed herein.

Endosomal release agents include imidazoles, poly or oligoimidazoles, PEIs, peptides, fusogenic peptides, polycarboxylates, polyacations, masked oligo or poly cations or anions, acetals, polyacetals, ketals/polyketyals, orthoesters, polymers with masked or unmasked cationic or anionic charges, dendrimers with masked or unmasked cationic or anionic charges.

PK modulator stands for pharmacokinetic modulator. PK modulator include lipophiles, bile acids, steroids, phospholipid analogues, peptides, protein binding agents, PEG, vitamins etc. Exemplary PK modulator include, but are not limited to, cholesterol, fatty acids, cholic acid, lithocholic acid, dialkylglycerides, diacylglyceride, phospholipids, sphingolipids, naproxen, ibuprofen, vitamin E, biotin etc. Oligonucleotides that comprise a number of phosphorothioate linkages are also known to bind to serum protein, thus short oligonucleotides, e.g. oligonucleotides of about 5 bases, 10 bases, 15 bases or 20 bases, comprising multiple of phosphorothioate linkages in the backbone are also amenable to the present invention as ligands (e.g. as PK modulating ligands).

In addition, aptamers that bind serum components (e.g. serum proteins) are also amenable to the present invention as PK modulating ligands.

Other ligands amenable to the invention are described in copending applications U.S. Ser. No. 10/916,185, filed Aug. 10, 2004; U.S. Ser. No. 10/946,873, filed Sep. 21, 2004; U.S. Ser. No. 10/833,934, filed Aug. 3, 2007; U.S. Ser. No. 11/115,989 filed Apr. 27, 2005 and U.S. Ser. No. 11/944,227 filed Nov. 21, 2007, which are incorporated by reference in their entireties for all purposes.

When two or more ligands are present, the ligands can all have same properties, all have different properties or some ligands have the same properties while others have different properties. For example, a ligand can have targeting properties, have endosomolytic activity or have PK modulating properties. In a preferred embodiment, all the ligands have different properties.

In one embodiment, at least one ligand is conjugated to the carrier oligomer. In one embodiment, at least one ligand is conjugated to the iRNA agent. In one embodiment, at least one ligand is conjugated to the carrier oligomer and at least one ligand is conjugated to the iRNA agent.

Conjugate moieties can be attached to any position of the iRNA agent or the carrier oligomer. In some embodiments, conjugate moieties can be attached to the terminus such as a 5′ or 3′ terminal residue of the iRNA agent or the carrier oligomer. Conjugate moieties can also be attached to internal residues of the iRNA agent or the carrier oligomer. For double-stranded iRNA agents and carrier oligomers, conjugate moieties can be attached to one or both strands. In some embodiments, a double-stranded iRNA agent contains a conjugate moiety attached to the sense strand. In other embodiments, a double-stranded iRNA agent contains a conjugate moiety attached to the antisense strand.

In some embodiments, conjugate moieties can be attached to nucleobases, sugar moieties, or internucleosidic linkages of nucleic acid molecules. Conjugation to purine nucleobases or derivatives thereof can occur at any position including, endocyclic and exocyclic atoms. In some embodiments, the 2-, 6-, 7-, or 8-positions of a purine nucleobase are attached to a conjugate moiety. Conjugation to pyrimidine nucleobases or derivatives thereof can also occur at any position. In some embodiments, the 2-, 5-, and 6-positions of a pyrimidine nucleobase can be substituted with a conjugate moiety. Conjugation to sugar moieties of nucleosides can occur at any carbon atom. Example carbon atoms of a sugar moiety that can be attached to a conjugate moiety include the 2′, 3′, and 5′ carbon atoms. The 1′ position can also be attached to a conjugate moiety, such as in an abasic residue. Internucleosidic linkages can also bear conjugate moieties. For phosphorus-containing linkages (e.g., phosphodiester, phosphorothioate, phosphorodithiotate, phosphoroamidate, and the like), the conjugate moiety can be attached directly to the phosphorus atom or to an O, N, or S atom bound to the phosphorus atom. For amine- or amide-containing internucleosidic linkages (e.g., PNA), the conjugate moiety can be attached to the nitrogen atom of the amine or amide or to an adjacent carbon atom.

There are numerous methods for preparing conjugates of oligomeric compounds. Generally, an oligomeric compound is attached to a conjugate moiety by contacting a reactive group (e.g., OH, SH, amine, carboxyl, aldehyde, and the like) on the oligomeric compound with a reactive group on the conjugate moiety. In some embodiments, one reactive group is electrophilic and the other is nucleophilic.

For example, an electrophilic group can be a carbonyl-containing functionality and a nucleophilic group can be an amine or thiol. Methods for conjugation of nucleic acids and related oligomeric compounds with and without linking groups are well described in the literature such as, for example, in Manoharan in Antisense Research and Applications, Crooke and LeBleu, eds., CRC Press, Boca Raton, Fla., 1993, Chapter 17, which is incorporated herein by reference in its entirety.

Representative United States patents that teach the preparation of oligonucleotide conjugates include, but are not limited to, U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,149,782; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928; 5,672,662; 5,688,941; 5,714,166; 6,153,737; 6,172,208; 6,300,319; 6,335,434; 6,335,437; 6,395,437; 6,444,806; 6,486,308; 6,525,031; 6,528,631; 6,559,279; each of which is herein incorporated by reference.

### Cleavable Linking Groups

A cleavable linking group is one which is sufficiently stable outside the cell, but which upon entry into a target cell is cleaved to release the two parts the linker is holding together. In a preferred embodiment, the cleavable linking group is cleaved at least 10 times or more, preferably at least 100 times faster in the target cell or under a first reference condition (which can, e.g., be selected to mimic or represent intracellular conditions) than in the blood of a subject, or under a second reference condition (which can, e.g., be selected to mimic or represent conditions found in the blood or serum).

Cleavable linking groups are susceptible to cleavage agents, e.g., pH, redox potential or the presence of degradative molecules. Generally, cleavage agents are more prevalent or found at higher levels or activities inside cells than in serum or blood. Examples of such degradative agents include: redox agents which are selected for particular substrates or which have no substrate specificity, including, e.g., oxidative or reductive enzymes or reductive agents such as mercaptans, present in cells, that can degrade a redox cleavable linking group by reduction; esterases; endosomes or agents that can create an acidic environment, e.g., those that result in a pH of five or lower; enzymes that can hydrolyze or degrade an acid cleavable linking group by acting as a general acid, peptidases (which can be substrate specific), and phosphatases.

A cleavable linkage group, such as a disulfide bond can be susceptible to pH. The pH of human serum is 7.4, while the average intracellular pH is slightly lower, ranging from about 7.1-7.3. Endosomes have a more acidic pH, in the range of 5.5-6.0, and lysosomes have an even more acidic pH at around 5.0. Some spacers will have a linkage group that is cleaved at a preferred pH, thereby releasing the iRNA agent from the carrier oligomer inside the cell, or into the desired compartment of the cell.

A spacer can include a linking group that is cleavable by a particular enzyme. The type of linking group incorporated into a spacer can depend on the cell to be targeted by the iRNA agent. For example, an iRNA agent that targets an mRNA in liver cells can be linked to the carrier oligomer through a spacer that includes an ester group. Liver cells are rich in esterases, and therefore the tether will be cleaved more efficiently in liver cells than in cell types that are not esterase-rich. Cleavage of the spacer releases the iRNA agent from the carrier oligomer, thereby potentially enhancing silencing activity of the mRNA agent. Other cell-types rich in esterases include cells of the lung, renal cortex, and testis.

Spacers that contain peptide bonds can be used when the iRNA agents are targeting cell types rich in peptidases, such as liver cells and synoviocytes. For example, an iRNA agent targeted to synoviocytes, such as for the treatment of an inflammatory disease (e.g., rheumatoid arthritis), can be linked to a carrier oligomer through spacer that comprises a peptide bond.

In general, the suitability of a candidate cleavable linking group can be evaluated by testing the ability of a degradative agent (or condition) to cleave the candidate linking group. It will also be desirable to also test the candidate cleavable linking group for the ability to resist cleavage in the blood or when in contact with other non-target tissue, e.g., tissue the iRNA agent would be exposed to when administered to a subject. Thus one can determine the relative susceptibility to cleavage between a first and a second condition, where the first is selected to be indicative of cleavage in a target cell and the second is selected to be indicative of cleavage in other tissues or biological fluids, e.g., blood or serum. The evaluations can be carried out in cell free systems, in cells, in cell culture, in organ or tissue culture, or in whole animals. It may be useful to make initial evaluations in cell-free or culture conditions and to confirm by further evaluations in whole animals. In preferred embodiments, useful candidate compounds are cleaved at least 2, 4, 10 or 100 times faster in the cell (or under in vitro conditions selected to mimic intracellular conditions) as compared to blood or serum (or under in vitro conditions selected to mimic extracellular conditions).

### Redox Cleavable Linking Groups

One class of cleavable linking groups are redox cleavable linking groups that are cleaved upon reduction or oxidation. An example of reductively cleavable linking group is a disulphide linking group (—S—S—). To determine if a candidate cleavable linking group is a suitable “reductively cleavable linking group,” or for example is suitable for use with a particular iRNA moiety and particular targeting agent one can look to methods described herein. For example, a candidate can be evaluated by incubation with dithiothreitol (DTT), or other reducing agent using reagents know in the art, which mimic the rate of cleavage which would be observed in a cell, e.g., a target cell. The candidates can also be evaluated under conditions which are selected to mimic

blood or serum conditions. In a preferred embodiment, candidate compounds are cleaved by at most 10% in the blood. In preferred embodiments, useful candidate compounds are degraded at least 2, 4, 10 or 100 times faster in the cell (or under in vitro conditions selected to mimic intracellular conditions) as compared to blood (or under in vitro conditions selected to mimic extracellular conditions). The rate of cleavage of candidate compounds can be determined using standard enzyme kinetics assays under conditions chosen to mimic intracellular media and compared to conditions chosen to mimic extracellular media.

### Phosphate-Based Cleavable Linking Groups

Phosphate-based linking groups are cleaved by agents that degrade or hydrolyze the phosphate group. An example of an agent that cleaves phosphate groups in cells are enzymes such as phosphatases in cells. Examples of phosphate-based linking groups are —O—P(O)(ORk)-O—, —O—P(S)(ORk)-O—, —O—P(S)(SRk)-O—, —S—P(O)(ORk)-O—, —O—P(O)(ORk)-S—, —S—P(O)(ORk)-S—, —O—P(S)(ORk)-S—, —S—P(S)(ORk)-O—, —O—P(O)(Rk)-O—, —O—P(S)(Rk)-O—, —S—P(O)(Rk)-O—, —S—P(S)(Rk)-O—, —S—P(O)(Rk)-S—, —O—P(S)(Rk)-S—. Preferred embodiments are —O—P(O)(OH)—O—, —O—P(S)(OH)—O—, —O—P(S)(SH)—O—, —S—P(O)(OH)—O—, —O—P(O)(OH)—S—, —S—P(O)(OH)—S—, —O—P(S)(OH)—S—, —S—P(S)(OH)—O—, —O—P(O)(H)—O—, —O—P(S)(H)—O—, —S—P(O)(H)—O—, —S—P(S)(H)—O—, —S—P(O)(H)—S—, —O—P(S)(H)—S—. A preferred embodiment is —O—P(O)(OH)—O—. These candidates can be evaluated using methods analogous to those described above.

### Acid Cleavable Linking Groups

Acid cleavable linking groups are linking groups that are cleaved under acidic conditions. In preferred embodiments acid cleavable linking groups are cleaved in an acidic environment with a pH of about 6.5 or lower (e.g., about 6.0, 5.5, 5.0, or lower), or by agents such as enzymes that can act as a general acid. In a cell, specific low pH organelles, such as endosomes and lysosomes can provide a cleaving environment for acid cleavable linking groups. Examples of acid cleavable linking groups include but are not limited to hydrazones, esters, and esters of amino acids. Acid cleavable groups can have the general formula —C═NN—, C(O)O, or —OC(O). A preferred embodiment is when the carbon attached to the oxygen of the ester (the alkoxy group) is an aryl group, substituted alkyl group, or tertiary alkyl group such as dimethyl pentyl or t-butyl. These candidates can be evaluated using methods analogous to those described above.

### Ester-Based Linking Groups

Ester-based linking groups are cleaved by enzymes such as esterases and amidases in cells. Examples of ester-based cleavable linking groups include but are not limited to esters of alkylene, alkenylene and alkynylene groups. Ester cleavable linking groups have the general formula —C(O)O—, or —OC(O)—. These candidates can be evaluated using methods analogous to those described above.

### Peptide-Based Cleaving Groups

Peptide-based linking groups are cleaved by enzymes such as peptidases and proteases in cells. Peptide-based cleavable linking groups are peptide bonds formed between amino acids to yield oligopeptides (e.g., dipeptides, tripeptides etc.) and polypeptides. Peptide-based cleavable groups do not include the amide group (—C(O)NH—). The amide group can be formed between any alkylene, alkenylene or alkynylene. A peptide bond is a special type of amide bond formed between amino acids to yield peptides and proteins. The peptide based cleavage group is generally limited to the peptide bond (i.e., the amide bond) formed between amino acids yielding peptides and proteins and does not include the entire amide functional group. Peptide cleavable linking groups have the general formula —NHCHR¹C(O)NHCHR²C(O)—, where R¹ and R² are the R groups of the two adjacent amino acids. These candidates can be evaluated using methods analogous to those described above.

iRNA Agent Structure

An “iRNA agent” as used herein, is an unmodified RNA, modified RNA, or nucleoside surrogate, all of which are defined herein. While numerous modified RNAs and nucleoside surrogates are described, preferred examples include those which have greater resistance to nuclease degradation than do unmodified RNAs. Preferred examples include those which have a 2′ sugar modification, a modification in a single strand overhang, preferably a 3′ single strand overhang, or, particularly if single stranded, a 5′ modification which includes one or more phosphate groups or one or more analogs of a phosphate group.

An “iRNA agent” as used herein, is an RNA agent which can, or which can be cleaved into an RNA agent which can, down regulate the expression of a target gene, preferably an endogenous or pathogen target RNA. While not wishing to be bound by theory, an iRNA agent may act by one or more of a number of mechanisms, including post-transcriptional cleavage of a target mRNA sometimes referred to in the art as RNAi, or pre-transcriptional or pre-translational mechanisms. An iRNA agent can include a single strand or can include more than one strands, e.g., it can be a double stranded iRNA agent. If the iRNA agent is a single strand it is particularly preferred that it include a 5′ modification which includes one or more phosphate groups or one or more analogs of a phosphate group. If the iRNA agent is double stranded the double stranded region can include more than two or more strands, e.g, two strands, e.g. three strands, in the double stranded region.

The iRNA agent should include a region of sufficient homology to the target gene, and be of sufficient length in terms of nucleotides, such that the iRNA agent, or a fragment thereof, can mediate down regulation of the target gene. (For ease of exposition the term nucleotide or ribonucleotide is sometimes used herein in reference to one or more monomeric subunits of an RNA agent. It will be understood herein that the usage of the term “ribonucleotide” or “nucleotide”, herein can, in the case of a modified RNA or nucleotide surrogate, also refer to a modified nucleotide, or surrogate replacement moiety at one or more positions.) Thus, the iRNA agent is or includes a region which is at least partially, and in some embodiments fully, complementary to the target RNA. It is not necessary that there be perfect complementarity between the iRNA agent and the target, but the correspondence must be sufficient to enable the iRNA agent, or a cleavage product thereof, to direct sequence specific silencing, e.g., by RNAi cleavage of the target RNA, e.g., mRNA.

An iRNA agent will often be modified or include nucleoside surrogates in addition to the ribose replacement modification subunit (RRMS). Single stranded regions of an iRNA agent will often be modified or include nucleoside surrogates, e.g., the unpaired region or regions of a hairpin structure, e.g., a region which links two complementary regions, can have modifications or nucleoside surrogates. Modification to stabilize one or more 3′- or 5′-terminus of an iRNA agent, e.g., against exonucleases, or to favor the antisense sRNA agent to enter into RISC are also favored. Modifications can include C3 (or C6, C7, C12) amino linkers, thiol linkers, carboxyl linkers, non-nucleotidic spacers (C3, C6, C9, C12, abasic, triethylene glycol, hexaethylene glycol), special biotin or fluorescein reagents that come as phosphoramidites and that have another DMT-protected hydroxyl group, allowing multiple couplings during RNA synthesis.

A “single strand iRNA agent” as used herein, is an iRNA agent which is made up of a single molecule. It may include a duplexed region, formed by intra-strand pairing, e.g., it may be, or include, a hairpin or pan-handle structure. Single strand iRNA agents are preferably antisense with regard to the target molecule. In preferred embodiments single strand iRNA agents are 5′-phosphorylated or include a phosphoryl analog at the 5′-terminus. 5′-phosphate modifications include those which are compatible with RISC mediated gene silencing. Suitable modifications include: 5′-monophosphate ((HO)2(O)P—O-5′); 5′-diphosphate ((HO)2(O)P—O—P(HO)(O)—O-5′); 5′-triphosphate ((HO)2(O)P—O—(HO)(O)P—O—P(HO)(O)—O-5′); 5′-guanosine cap (7-methylated or non-methylated) (7m-G-O-5′-(HO)(O)P—O—(HO)(O)P—O—P(HO)(O)—O-5′); 5′-adenosine cap (Appp), and any modified or unmodified nucleotide cap structure (N—O-5′-(HO)(O)P—O—(HO)(O)P—O—P(HO)(O)—O-5′); 5′-monothiophosphate (phosphorothioate; (HO)2(S)P—O-5); 5′-monodithiophosphate (phosphorodithioate; (HO)(HS)(S)P—O-5′), 5′-phosphorothiolate ((HO)2(O)P—S-5′); any additional combination of oxygen/sulfur replaced monophosphate, diphosphate and triphosphates (e.g. 5′-alpha-thiotriphosphate, 5′-gamma-thiotriphosphate, etc.), 5′-phosphoramidates ((HO)2(O)P—NH-5′, (HO)(NH2)(O)P—O-5′), 5′-alkylphosphonates (R=alkyl=methyl, ethyl, isopropyl, propyl, etc., e.g. RP(OH)(O)—O-5′-, (OH)2(O)P-5′-CH2-), 5′-alkyletherphosphonates (R=alkylether=methoxymethyl (MeOCH2-), ethoxymethyl, etc., e.g. RP(OH)(O)—O-5′-). (These modifications can also be used with the antisense strand of a multi-strand iRNA agent.)

A “multi-strand iRNA agent” as used herein, is an iRNA agent which comprises two or more strands, for example a double-stranded iRNA agent. The strands form duplexed regions and may include a hairpin, pan-handle structure, loop or bulges. At least one strand of the iRNA agent is preferably antisense with regard to the target molecule.

It may be desirable to modify only one, only two or all strands of a multi-strand iRNA agent. In some cases they will have the same modification or the same class of modification but in other cases the different strand will have different modifications, e.g., in some cases it is desirable to modify only one strand. It may be desirable to modify only some strands, e.g., to inactivate them, e.g., strands can be modified in order to inactivate them and prevent formation of an active iRNA/protein or RISC. This can be accomplished by a modification which prevents 5′-phosphorylation of the strands, e.g., by modification with a 5′-O-methyl ribonucleotide (see Nykänen et al., (2001) ATP requirements and small interfering RNA structure in the RNA interference pathway. Cell 107, 309-321.) Other modifications which prevent phosphorylation can also be used, e.g., simply substituting the 5′-OH by H rather than O-Me. Alternatively, a large bulky group may be added to the 5′-phosphate turning it into a phosphodiester linkage, though this may be less desirable as phosphodiesterases can cleave such a linkage and release a functional iRNA 5′-end. Antisense strand modifications include 5′-phosphorylation as well as any of the other 5′ modifications discussed herein, particularly the 5′ modifications discussed above in the section on single stranded iRNA molecules.

In some cases, the different strands will include different modifications. Multiple different modifications can be included on each of the strands. The modifications on a given strand may differ from each other, and may also differ from the various modifications on other strands. For example, one strand may have a modification, e.g., a modification described herein, and a different strand may have a different modification, e.g., a different modification described herein. In other cases, one strand may have two or more different modifications, and the another strand may include a modification that differs from the at least two modifications on the other strand.

It is preferred that the strands be chosen such that the iRNA agent includes a single strand or unpaired region at one or both ends of the molecule. Thus, an iRNA agent contains two or more strands, preferable paired to contain an overhang, e.g., one or two 5′ or 3′ overhangs but preferably a 3′ overhang of 2-3 nucleotides. Most embodiments will have a 3′ overhang. Preferred iRNA agents will have single-stranded overhangs, preferably 3′ overhangs, of 1 or preferably 2 or 3 nucleotides in length at each end. The overhangs can be the result of one strand being longer than the other, or the result of two strands of the same length being staggered.

Preferred lengths for the duplexed regions between the strands are between 6 and 30 nucleotides in length. The preferred duplexed regions are between 15 and 30, most preferably 18, 19, 20, 21, 22, and 23 nucleotides in length. Other preferred duplexed regions are between 6 and 20 nucleotides, most preferably 6, 7, 8, 9, 10, 11 and 12 nucleotides in length. In multi-strand iRNA agents different duplexes formed may have different lengths, e.g. duplexed region formed between strand A and B may have a different length than duplexed region formed between strand A and C. In iRNA agents comprising more than two strands duplexed agents can resemble in length and structure the natural Dicer processed products from long dsRNAs. Embodiments in which the two or more strands of the iRNA agent are linked, e.g., covalently linked are also included. Hairpins or other single strand structures which provide the required double stranded region, and preferably a 3′ overhang are also within the invention.

As nucleic acids are polymers of subunits or monomers, many of the modifications described below occur at a position which is repeated within a nucleic acid, e.g., a modification of a base, or a phosphate moiety, or the non-bridging oxygen of a phosphate moiety. In some cases the modification will occur at all of the subject positions in the nucleic acid but in many, and in fact in most cases it will not. By way of example, a modification may only occur at a 3′ or 5′ terminal position, may only occur in the internal unpaired region, may only occur in a terminal regions, e.g. at a position on a terminal nucleotide or in the last 2, 3, 4, 5, or 10 nucleotides of a strand. A modification may occur in a double strand region, a single strand region, or in both. A modification may occur only in the double strand region of an RNA agent or may only occur in a single strand region of an RNA agent. E.g., a phosphorothioate modification at a non-bridging oxygen position may only occur at one or both termini, may only occur in a terminal regions, e.g., at a position on a terminal nucleotide or in the last 2, 3, 4, 5, or 10 nucleotides of a strand, or may occur in double strand and single strand regions, particularly at termini. The 5′ end or ends can be phosphorylated.

In some embodiments it is particularly preferred, e.g., to enhance stability, to include particular bases in overhangs, or to include modified nucleotides or nucleotide surrogates, in single strand overhangs, e.g., in a 5′ or 3′ overhang, or in both. E.g., it can be desirable to include purine nucleotides in overhangs. In some embodiments all or some of the bases in a 3′ or 5′ overhang will be modified, e.g., with a modification described herein. Modifications can include, e.g., the use of modifications at the 2′ OH group of the ribose sugar, e.g., the use of deoxyribonucleotides, e.g., deoxythymidine, instead of ribonucleotides, and modifications in the phosphate group, e.g., phosphothioate modifications. Overhangs need not be homologous with the target sequence.

Specific modifications are discussed in more detail below. Although, the modifications herein are described in context of an iRNA agent, these modifications are also amenable in modifying the carrier oligonucleotides of the invention.

### The Phosphate Group

The phosphate group is a negatively charged species. The charge is distributed equally over the two non-bridging oxygen atoms. However, the phosphate group can be modified by replacing one of the oxygens with a different substituent. One result of this modification to RNA phosphate backbones can be increased resistance of the oligoribonucleotide to nucleolytic breakdown. Thus while not wishing to be bound by theory, it can be desirable in some embodiments to introduce alterations which result in either an uncharged linker or a charged linker with unsymmetrical charge distribution.

Examples of modified phosphate groups include phosphorothioate, phosphoroselenates, borano phosphates, borano phosphate esters, hydrogen phosphonates, phosphoroamidates, alkyl or aryl phosphonates and phosphotriesters. Phosphorodithioates have both non-bridging oxygens replaced by sulfur. The phosphorus center in the phosphorodithioates is achiral which precludes the formation of oligoribonucleotides diastereomers. Diastereomer formation can result in a preparation in which the individual diastereomers exhibit varying resistance to nucleases. Further, the hybridization affinity of RNA containing chiral phosphate groups can be lower relative to the corresponding unmodified RNA species. Thus, while not wishing to be bound by theory, modifications to both non-bridging oxygens, which eliminate the chiral center, e.g. phosphorodithioate formation, may be desirable in that they cannot produce diastereomer mixtures. Thus, the non-bridging oxygens can be independently any one of S, Se, B, C, H, N, or OR (R is alkyl or aryl). Replacement of the non-bridging oxygens with sulfur is preferred.

The phosphate linker can also be modified by replacement of bridging oxygen, (i.e. oxygen that links the phosphate to the nucleoside), with nitrogen (bridged phosphoroamidates), sulfur (bridged phosphorothioates) and carbon (bridged methylenephosphonates). The replacement can occur at the either linking oxygen or at both the linking oxygens. When the bridging oxygen is the 3′-oxygen of a nucleoside, replacement with carbon is preferred. When the bridging oxygen is the 5′-oxygen of a nucleoside, replacement with nitrogen is preferred.

### Replacement of the Phosphate Group

The phosphate group can be replaced by non-phosphorus containing connectors. While not wishing to be bound by theory, it is believed that since the charged phosphodiester group is the reaction center in nucleolytic degradation, its replacement with neutral structural mimics should impart enhanced nuclease stability. Again, while not wishing to be bound by theory, it can be desirable, in some embodiment, to introduce alterations in which the charged phosphate group is replaced by a neutral moiety.

Examples of moieties which can replace the phosphate group include siloxane, carbonate, carboxymethyl, carbamate, amide, thioether, ethylene oxide linker, sulfonate, sulfonamide, thioformacetal, formacetal, oxime, methyleneimino, methylenemethylimino, methylenehydrazo, methylenedimethylhydrazo and methyleneoxymethylimino. Preferred replacements include the methylenecarbonylamino and methylenemethylimino groups.

Modified phosphate linkages where at least one of the oxygens linked to the phosphate has been replaced or the phosphate group has been replaced by a non-phosphorous group, are also referred to as “non phosphodiester backbone linkage.”

### Replacement of Ribophosphate Backbone

Oligonucleotide-mimicking scaffolds can also be constructed wherein the phosphate linker and ribose sugar are replaced by nuclease resistant nucleoside or nucleotide surrogates. While not wishing to be bound by theory, it is believed that the absence of a repetitively charged backbone diminishes binding to proteins that recognize polyanions (e.g. nucleases). Again, while not wishing to be bound by theory, it can be desirable in some embodiment, to introduce alterations in which the bases are tethered by a neutral surrogate backbone. Examples include the mophilino, cyclobutyl, pyrrolidine and peptide nucleic acid (PNA) nucleoside surrogates. A preferred surrogate is a PNA surrogate.

### The Sugar Group

A modified RNA can include modification of all or some of the sugar groups of the ribonucleic acid. E.g., the 2′ hydroxyl group (OH) can be modified or replaced with a number of different “oxy” or “deoxy” substituents. While not being bound by theory, enhanced stability is expected since the hydroxyl can no longer be deprotonated to form a 2′-alkoxide ion. The 2′-alkoxide can catalyze degradation by intramolecular nucleophilic attack on the linker phosphorus atom. Again, while not wishing to be bound by theory, it can be desirable to some embodiments to introduce alterations in which alkoxide formation at the 2′ position is not possible.

Examples of “oxy”-2′ hydroxyl group modifications include alkoxy or aryloxy (OR, e.g., R═H, alkyl, cycloalkyl, aryl, aralkyl, heteroaryl or sugar); polyethyleneglycols (PEG), O(CH₂CH₂O)ₙCH₂CH₂OR; “locked” nucleic acids (LNA) in which the 2′ hydroxyl is connected, e.g., by a methylene bridge, to the 4′ carbon of the same ribose sugar; O-AMINE (AMINE=NH₂; alkylamino, dialkylamino, heterocyclyl, arylamino, diaryl amino, heteroaryl amino, or diheteroaryl amino, ethylene diamine, polyamino) and aminoalkoxy, O(CH₂)ₙAMINE, (e.g., AMINE=NH₂; alkylamino, dialkylamino, heterocyclyl, arylamino, diaryl amino, heteroaryl amino, or diheteroaryl amino, ethylene diamine, polyamino). It is noteworthy that oligonucleotides containing only the methoxyethyl group (MOE), (OCH₂CH₂OCH₃, a PEG derivative), exhibit nuclease stabilities comparable to those modified with the robust phosphorothioate modification.

“Deoxy” modifications include hydrogen (i.e. deoxyribose sugars, which are of particular relevance to the overhang portions of partially ds RNA); halo (e.g., fluoro); amino (e.g. NH₂; alkylamino, dialkylamino, heterocyclyl, arylamino, diaryl amino, heteroaryl amino, diheteroaryl amino, or amino acid); NH(CH₂CH₂NH)ₙCH₂CH₂-AMINE (AMINE=NH₂; alkylamino, dialkylamino, heterocyclyl, arylamino, diaryl amino, heteroaryl amino, or diheteroaryl amino), —NHC(O)R (R=alkyl, cycloalkyl, aryl, aralkyl, heteroaryl or sugar), cyano; mercapto; alkyl-thio-alkyl; thioalkoxy; and alkyl, cycloalkyl, aryl, alkenyl and alkynyl, which may be optionally substituted with e.g., an amino functionality. Preferred substitutents are 2′-methoxyethyl, 2′-OCH3,2′-O-allyl, 2′-C-allyl, and 2′-fluoro.

The sugar group can also contain one or more carbons that possess the opposite stereochemical configuration than that of the corresponding carbon in ribose. Thus, a modified RNA can include nucleotides containing e.g., arabinose, as the sugar. Modified RNAs can also include “abasic” sugars, which lack a nucleobase at C-1′. These abasic sugars can also be further contain modifications at one or more of the constituent sugar atoms.

To maximize nuclease resistance, the 2′ modifications can be used in combination with one or more phosphate linker modifications (e.g., phosphorothioate). The so-called “chimeric” oligonucleotides are those that contain two or more different modifications.

The modification can also entail the wholesale replacement of a ribose structure with another entity at one or more sites in the iRNA agent.

### Terminal Modifications

The 3′ and 5′ ends of an oligonucleotide can be modified. Such modifications can be at the 3′ end, 5′ end or both ends of the molecule. They can include modification or replacement of an entire terminal phosphate or of one or more of the atoms of the phosphate group. E.g., the 3′ and 5′ ends of an oligonucleotide can be conjugated to other functional molecular entities such as labeling moieties, e.g., fluorophores (e.g., pyrene, TAMRA, fluorescein, Cy3 or Cy5 dyes) or protecting groups (based e.g., on sulfur, silicon, boron or ester). The functional molecular entities can be attached to the sugar through a phosphate group and/or a spacer. The terminal atom of the spacer can connect to or replace the linking atom of the phosphate group or the C-3′ or C-5′ O, N, S or C group of the sugar. Alternatively, the spacer can connect to or replace the terminal atom of a nucleotide surrogate (e.g., PNAs). These spacers or linkers can include e.g., —(CH₂)ₙ—, —(CH₂)ₙN—, —(CH₂)ₙO—, —(CH₂)ₙS—, O(CH₂CH₂O)ₙCH₂CH₂OH (e.g., n=3 or 6), abasic sugars, amide, carboxy, amine, oxyamine, oxyimine, thioether, disulfide, thiourea, sulfonamide, or morpholino, or biotin and fluorescein reagents. When a spacer/phosphate-functional molecular entity-spacer/phosphate array is interposed between two strands of iRNA agents, this array can substitute for a hairpin RNA loop in a hairpin-type RNA agent. The 3′ end can be an —OH group. While not wishing to be bound by theory, it is believed that conjugation of certain moieties can improve transport, hybridization, and specificity properties. Again, while not wishing to be bound by theory, it may be desirable to introduce terminal alterations that improve nuclease resistance. Other examples of terminal modifications include dyes, intercalating agents (e.g. acridines), cross-linkers (e.g. psoralene, mitomycin C), porphyrins (TPPC4, texaphyrin, Sapphyrin), polycyclic aromatic hydrocarbons (e.g., phenazine, dihydrophenazine), artificial endonucleases (e.g. EDTA), lipophilic carriers (e.g., cholesterol, cholic acid, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid, O3-(oleoyl)lithocholic acid, O3-(oleoyl)cholenic acid, dimethoxytrityl, or phenoxazine) and peptide conjugates (e.g., antennapedia peptide, Tat peptide), alkylating agents, phosphate, amino, mercapto, PEG (e.g., PEG-40K), MPEG, [MPEG]₂, polyamino, alkyl, substituted alkyl, radiolabeled markers, enzymes, haptens (e.g. biotin), transport/absorption facilitators (e.g., aspirin, vitamin E, folic acid), synthetic ribonucleases (e.g., imidazole, bisimidazole, histamine, imidazole clusters, acridine-imidazole conjugates, Eu3+ complexes of tetraazamacrocycles).

Terminal modifications can be added for a number of reasons, including as discussed elsewhere herein to modulate activity or to modulate resistance to degradation.

Terminal modifications useful for modulating activity include modification of the 5′ end with phosphate or phosphate analogs. E.g., in preferred embodiments iRNA agents, especially antisense strands, are 5′ phosphorylated or include a phosphoryl analog at the 5′ prime terminus. 5′-phosphate modifications include those which are compatible with RISC mediated gene silencing. Suitable modifications include: 5′-monophosphate ((HO)2(O)P—O-5′); 5′-diphosphate ((HO)2(O)P—O—P(HO)(O)—O-5′); 5′-triphosphate ((HO)2(O)P—O—(HO)(O)P—O—P(HO)(O)—O-5′); 5′-guanosine cap (7-methylated or non-methylated) (7m-G-O-5′-(HO)(O)P—O—(HO)(O)P—O—P(HO)(O)—O-5′); 5′-adenosine cap (Appp), and any modified or unmodified nucleotide cap structure (N—O-5′-(HO)(O)P—O—(HO)(O)P—O—P(HO)(O)—O-5′); 5′-monothiophosphate (phosphorothioate; (HO)2(S)P—O-5); 5′-monodithiophosphate (phosphorodithioate; (HO)(HS)(S)P—O-5′), 5′-phosphorothiolate ((HO)2(O)P—S-5′); any additional combination of oxygen/sulfur replaced monophosphate, diphosphate and triphosphates (e.g. 5′-alpha-thiotriphosphate, 5′-gamma-thiotriphosphate, etc.), 5′-phosphoramidates ((HO)2(O)P—NH-5′, (HO)(NH2)(O)P—O-5′), 5′-alkylphosphonates (R=alkyl=methyl, ethyl, isopropyl, propyl, etc., e.g. RP(OH)(O)—O-5′-, (OH)2(O)P-5′-CH2-), 5′-alkyletherphosphonates (R=alkylether=methoxymethyl (MeOCH2-), ethoxymethyl, etc., e.g. RP(OH)(O)—O-5′-).

Terminal modifications can also be useful for monitoring distribution, and in such cases the preferred groups to be added include fluorophores, e.g., fluorscein or an Alexa dye, e.g., Alexa 488. Terminal modifications can also be useful for enhancing uptake, useful modifications for this include cholesterol. Terminal modifications can also be useful for cross-linking an RNA agent to another moiety; modifications useful for this include mitomycin C.

### The Bases

Adenine, guanine, cytosine and uracil are the most common bases found in RNA. These bases can be modified or replaced to provide RNA's having improved properties. E.g., nuclease resistant oligoribonucleotides can be prepared with these bases or with synthetic and natural nucleobases (e.g., inosine, thymine, xanthine, hypoxanthine, nubularine, isoguanisine, or tubercidine) and any one of the above modifications. Alternatively, substituted or modified analogs of any of the above bases, e.g., “unusual bases”, “modified bases”, “non-natural bases” and “universal bases” described herein, can be employed. Examples include without limitation 2-aminoadenine, 6-methyl and other alkyl derivatives of adenine and guanine, 2-propyl and other alkyl derivatives of adenine and guanine, 5-halouracil and cytosine, 5-propynyl uracil and cytosine, 6-azo uracil, cytosine and thymine, 5-uracil (pseudouracil), 4-thiouracil, 5-halouracil, 5-(2-aminopropyl)uracil, 5-amino allyl uracil, 8-halo, amino, thiol, thioalkyl, hydroxyl and other 8-substituted adenines and guanines, 5-trifluoromethyl and other 5-substituted uracils and cytosines, 7-methylguanine, 5-substituted pyrimidines, 6-azapyrimidines and N-2, N-6 and O-6 substituted purines, including 2-aminopropyladenine, 5-propynyluracil and 5-propynylcytosine, dihydrouracil, 3-deaza-5-azacytosine, 2-aminopurine, 5-alkyluracil, 7-alkylguanine, 5-alkyl cytosine, 7-deazaadenine, N6, N6-dimethyladenine, 2,6-diaminopurine, 5-amino-allyl-uracil, N3-methyluracil, substituted 1,2,4-triazoles, 2-pyridinone, 5-nitroindole, 3-nitropyrrole, 5-methoxyuracil, uracil-5-oxyacetic acid, 5-methoxycarbonylmethyluracil, 5-methyl-2-thiouracil, 5-methoxycarbonylmethyl-2-thiouracil, 5-methylaminomethyl-2-thiouracil, 3-(3-amino-3-carboxypropyl)uracil, 3-methylcytosine, 5-methylcytosine, N⁴-acetyl cytosine, 2-thiocytosine, N6-methyladenine, N6-isopentyladenine, 2-methylthio-N6-isopentenyladenine, N-methylguanines, or O-alkylated bases. Further purines and pyrimidines include those disclosed in U.S. Pat. No. 3,687,808, those disclosed in the Concise Encyclopedia Of Polymer Science And Engineering, pages 858-859, Kroschwitz, J. I., ed. John Wiley & Sons, 1990, and those disclosed by Englisch et al., Angewandte Chemie, International Edition, 1991, 30, 613.

Generally, base changes are less preferred for promoting stability, but they can be useful for other reasons, e.g., some, e.g., 2,6-diaminopurine and 2 amino purine, are fluorescent. Modified bases can reduce target specificity. This should be taken into consideration in the design of iRNA agents.

### Cationic Groups

Modifications can also include attachment of one or more cationic groups to the sugar, base, and/or the phosphorus atom of a phosphate or modified phosphate backbone moiety. A cationic group can be attached to any atom capable of substitution on a natural, unusual or universal base. A preferred position is one that does not interfere with hybridization, i.e., does not interfere with the hydrogen bonding interactions needed for base pairing. A cationic group can be attached e.g., through the C2′ position of a sugar or analogous position in a cyclic or acyclic sugar surrogate. Cationic groups can include e.g., protonated amino groups, derived from e.g., O-AMINE (AMINE=NH₂; alkylamino, dialkylamino, heterocyclyl, arylamino, diaryl amino, heteroaryl amino, or diheteroaryl amino, ethylene diamine, polyamino); aminoalkoxy, e.g., O(CH₂)ₙAMINE, (e.g., AMINE=NH₂; alkylamino, dialkylamino, heterocyclyl, arylamino, diaryl amino, heteroaryl amino, or diheteroaryl amino, ethylene diamine, polyamino); amino (e.g. NH₂; alkylamino, dialkylamino, heterocyclyl, arylamino, diaryl amino, heteroaryl amino, diheteroaryl amino, or amino acid); or NH(CH₂CH₂NH)ₙCH₂CH₂-AMINE (AMINE=NH₂; alkylamino, dialkylamino, heterocyclyl, arylamino, diaryl amino, heteroaryl amino, or diheteroaryl amino).

Exemplary Modifications and Placement within an iRNA Agent

Some modifications may preferably be included on an iRNA agent at a particular location, e.g., at an internal position of a strand, or on the 5′ or 3′ end of a strand of an iRNA agent. A preferred location of a modification on an iRNA agent, may confer preferred properties on the agent. For example, preferred locations of particular modifications may confer optimum gene silencing properties, or increased resistance to endonuclease or exonuclease activity. A modification described herein and below may be the sole modification, or the sole type of modification included on multiple ribonucleotides, or a modification can be combined with one or more other modifications described herein and below. For example, a modification on one strand of a multi-strand iRNA agent can be different than a modification on another strand of the multi-strand iRNA agent. Similarly, two different modifications on one strand can differ from a modification on a different strand of the iRNA agent. Other additional unique modifications, without limitation, can be incorporates into strands of the iRNA agent.

An iRNA agent may include a backbone modification to any nucleotide on an iRNA strand. For example, an iRNA agent may include a phosphorothioate linkage or P-alkyl modification in the linkages between one or more nucleotides of an iRNA agent. The nucleotides can be terminal nucleotides, e.g., nucleotides at the last position of a sense or antisense strand, or internal nucleotides.

An iRNA agent can include a sugar modification, e.g., a 2′ or 3′ sugar modification. Exemplary sugar modifications include, for example, a 2 -O-methylated nucleotide, a 2′-deoxy nucleotide, (e.g., a 2′-deoxyfluoro nucleotide), a 2-O-methoxyethyl nucleotide, a 2′-O-NMA, a 2′-DMAEOE, a 2′-aminopropyl, 2′-hydroxy, or a 2′-ara-fluoro or a locked nucleic acid (LNA), extended nucleic acid (ENA), hexose nucleic acid (HNA), or cyclohexene nucleic acid (CeNA). A 2′ modification is preferably 2-OMe, and more preferably, 2′-deoxyfluoro. When the modification is 2% OMe, the modification is preferably on the sense strands. When the modification is a 2′-fluoro, and the modification may be on any strand of the iRNA agent. A 2′-ara-fluoro modification will preferably be on the sense strands of the iRNA agent. An iRNA agent may include a 3′ sugar modification, e.g., a 3-OMe modification. Preferably a 3-OMe modification is on the sense strand of the iRNA agent.

An iRNA agent may include a 5′-methyl-pyrimidine (e.g., a 5′-methyl-uridine modification or a 5′-methyl-cytodine) modification.

The modifications described herein can be combined onto a single iRNA agent. For example, an iRNA agent may have a phosphorothioate linkage and a 2′ sugar modification, e.g., a 2′-OMe or 2′-F modification. In another example, an iRNA agent may include at least one 5-Me-pyrimidine and a 2′-sugar modification, e.g., a 2′-F or 2% OMe modification.

An iRNA agent may include a nucleobase modification, such as a cationic modification, such as a 3′-abasic cationic modification. The cationic modification can be e.g., an alkylamino-dT (e.g., a C6 amino-dT), an allylamino conjugate, a pyrrolidine conjugate, a pthalamido, a porphyrin, or a hydroxyprolinol conjugate, on one or more of the terminal nucleotides of the iRNA agent. When an alkylamino-dT conjugate is attached to the terminal nucleotide of an iRNA agent, the conjugate is preferably attached to the 3′ end of the sense or antisense strand of an iRNA agent. When a pyrrolidine linker is attached to the terminal nucleotide of an iRNA agent, the linker is preferably attached to the 3′- or 5′-end of the sense strand, or the 3′-end of the antisense strand. When a pyrrolidine linker is attached to the terminal nucleotide of an iRNA agent, the linker is preferably on the 3′- or 5′-end of the sense strand, and not on the 5′-end of the antisense strand.

An iRNA agent may include at least one conjugate, such as a lipophile, a terpene, a protein binding agent, a vitamin, a carbohydrate, or a peptide. For example, the conjugate can be naproxen, nitroindole (or another conjugate that contributes to stacking interactions), folate, ibuprofen, or a C5 pyrimidine linker. The conjugate can also be a glyceride lipid conjugate (e.g., a dialkyl glyceride derivatives), vitamin E conjugate, or a thio-cholesterol. In generally, and except where noted to the contrary below, when a conjugate is on the terminal nucleotide of a sense or antisense strand, the conjugate is preferably on the 5′ or 3′ end of the sense strand or on the 5′ end of the antisense strand, and preferably the conjugate is not on the 3′ end of the antisense strand.

When the conjugate is naproxen, and the conjugate is on the terminal nucleotide of a sense or antisense strand, the conjugate is preferably on the 5′ or 3′ end of the sense or antisense strands. When the conjugate is cholesterol, and the conjugate is on the terminal nucleotide of a sense or antisense strand, the cholesterol conjugate is preferably on the 5′ or 3′ end of the sense strand and preferably not present on the antisense strand. Cholesterol may be conjugated to the iRNA agent by a pyrrolidine linker, serinol linker, hydroxyprolinol linker, or disulfide linkage. A dU-cholesterol conjugate may also be conjugated to the iRNA agent by a disulfide linkage. When the conjugate is cholanic acid, and the conjugate is on the terminal nucleotide of a sense or antisense strand, the cholanic acid is preferably attached to the 5′ or 3′ end of the sense strand, or the 3′ end of the antisense strand. In one embodiment, the cholanic acid is attached to the 3′ end of the sense strand and the 3′ end of the antisense strand.

One or more nucleotides of an iRNA agent may have a 2′-5′ linkage. Preferably, the 2′-5′ linkage is on the sense strand. When the 2′-5′ linkage is on the terminal nucleotide of an iRNA agent, the 2′-5′ linkage occurs on the 5′ end of the sense strand. The iRNA agent may include an L-sugar, preferably on the sense strand, and not on the antisense strand.

The iRNA agent may include a methylphosphonate modification. When the methylphosphonate is on the terminal nucleotide of an iRNA agent, the methylphosphonate is at the 3′ end of the sense or antisense strands of the iRNA agent.

An iRNA agent may be modified by replacing one or more ribonucleotides with deoxyribonucleotides. Preferably, adjacent deoxyribonucleotides are joined by phosphorothioate linkages, and the iRNA agent does not include more than four consecutive deoxyribonucleotides on the sense or the antisense strands.

An iRNA agent may include a difluorotoluoyl (DFT) modification, e.g., 2,4-difluorotoluoyl uracil, or a guanidine to inosine substitution.

The iRNA agent may include at least one 5′-uridine-adenine-3′ (5′-UA-3′) dinucleotide wherein the uridine is a 2′-modified nucleotide, or a terminal 5′-uridine-guanine-3′ (5′-UG-3′) dinucleotide, wherein the 5′-uridine is a 2′-modified nucleotide, or a terminal 5′-cytidine-adenine-3′ (5′-CA-3′) dinucleotide, wherein the 5′-cytidine is a 2′-modified nucleotide, or a terminal 5′-uridine-uridine-3′ (5′-UU-3′) dinucleotide, wherein the 5′-uridine is a 2′-modified nucleotide, or a terminal 5′-cytidine-cytidine-3′ (5′-CC-3′) dinucleotide, wherein the 5′-cytidine is a 2′-modified nucleotide, or a terminal 5′-cytidine-uridine-3′ (5′-CU-3′) dinucleotide, wherein the 5′-cytidine is a 2′-modified nucleotide, or a terminal 5′-uridine-cytidine-3′ (5′-UC-3′) dinucleotide, wherein the 5′-uridine is a 2′-modified nucleotide. The chemically modified nucleotide in the iRNA agent may be a 2-O-methylated nucleotide. In some embodiments, the modified nucleotide can be a 2′-deoxy nucleotide, a 2′-deoxyfluoro nucleotide, a 2-O-methoxyethyl nucleotide, a 2′-O-NMA, a 2′-DMAEOE, a 2′-aminopropyl, 2′-hydroxy, or a 2′-ara-fluoro, or a locked nucleic acid (LNA), extended nucleic acid (ENA), hexose nucleic acid (HNA), or cyclohexene nucleic acid (CeNA). The iRNA agents including these modifications are particularly stabilized against exonuclease activity, when the modified dinucleotide occurs on a terminal end of the sense or antisense strand of an iRNA agent, and are otherwise particularly stabilized against endonuclease activity.

An iRNA agent may have a single overhang, e.g., one end of the iRNA agent has a 3′ or 5′ overhang and the other end of the iRNA agent is a blunt end, or the iRNA agent may have a double overhang, e.g., both ends of the iRNA agent have a 3′ or 5′ overhang, such as a dinucleotide overhang. In another alternative, both ends of the iRNA agent may have blunt ends. The unpaired nucleotides may have at least one phosphorothioate dinucleotide linkage, and at least one of the unpaired nucleotides may be chemically modified in the 2′-position. The double strand region of the iRNA agent may include phosphorothioate dinucleotide linkages on one or both of the sense and antisense strands. Various strands of the multi-strand iRNA agent may be connected with a linker, e.g., a chemical linker such as hexaethylene glycol linker, a poly-(oxyphosphinico-oxy-1,3-propandiol) linker, an allyl linker, or a polyethylene glycol linker.

### Nuclease Resistant Monomers

An iRNA agent can include monomers which have been modified so as to inhibit degradation, e.g., by nucleases, e.g., endonucleases or exonucleases, found in the body of a subject. These monomers are referred to herein as NRMs, or nuclease resistance promoting monomers or modifications. In many cases these modifications will modulate other properties of the iRNA agent as well, e.g., the ability to interact with a protein, e.g., a transport protein, e.g., serum albumin, or a member of the RISC(RNA-induced Silencing Complex), or the ability of the first and second sequences to form a duplex with one another or to form a duplex with another sequence, e.g., a target molecule.

While not wishing to be bound by theory, it is believed that modifications of the sugar, base, and/or phosphate backbone in an iRNA agent can enhance endonuclease and exonuclease resistance, and can enhance interactions with transporter proteins and one or more of the functional components of the RISC complex. Preferred modifications are those that increase exonuclease and endonuclease resistance and thus prolong the half-life of the iRNA agent prior to interaction with the RISC complex, but at the same time do not render the iRNA agent resistant to endonuclease activity in the RISC complex. Again, while not wishing to be bound by any theory, it is believed that placement of the modifications at or near the 3′ and/or 5′ end of antisense strands can result in iRNA agents that meet the preferred nuclease resistance criteria delineated above. Again, still while not wishing to be bound by any theory, it is believed that placement of the modifications at e.g., the middle of a sense strand can result in iRNA agents that are relatively less likely to undergo off-targeting.

Modifications described herein can be incorporated into any RNA and RNA-like molecule described herein, e.g., an iRNA agent, a carrier oligonucleotide. An iRNA agent may include a duplex comprising a hybridized sense and antisense strand, in which the antisense strand and/or the sense strand may include one or more of the modifications described herein. The anti sense strand may include modifications at the 3′ end and/or the 5′ end and/or at one or more positions that occur 1-6 (e.g., 1-5, 1-4, 1-3, 1-2) nucleotides from either end of the strand. The sense strand may include modifications at the 3′ end and/or the 5′ end and/or at any one of the intervening positions between the two ends of the strand. The iRNA agent may also include a duplex comprising two hybridized antisense strands. The first and/or the second antisense strand may include one or more of the modifications described herein. Thus, one and/or both antisense strands may include modifications at the 3′ end and/or the 5′ end and/or at one or more positions that occur 1-6 (e.g., 1-5, 1-4, 1-3, 1-2) nucleotides from either end of the strand. Particular configurations are discussed below.

Modifications that can be useful for producing iRNA agents that meet the preferred nuclease resistance criteria delineated above can include one or more of the following chemical and/or stereochemical modifications of the sugar, base, and/or phosphate backbone:
(i) chiral (Sₚ) thioates. Thus, preferred NRMs include nucleotide dimers with an enriched or pure for a particular chiral form of a modified phosphate group containing a heteroatom at the nonbridging position, e.g., Sp or Rp, where this is the position normally occupied by the oxygen. The heteroatom can be S, Se, Nr₂, or Br₃. When the heteroatom is S, enriched or chirally pure Sp linkage is preferred. Enriched means at least 70, 80, 90, 95, or 99% of the preferred form. Such NRMs are discussed in more detail below;
(ii) attachment of one or more cationic groups to the sugar, base, and/or the phosphorus atom of a phosphate or modified phosphate backbone moiety. Thus, preferred NRMs include monomers at the terminal position derivatized at a cationic group. As the 5′ end of an antisense sequence should have a terminal —OH or phosphate group this NRM is preferably not used at the 5′ end of an anti-sense sequence. The group should be attached at a position on the base which minimizes interference with H bond formation and hybridization, e.g., away form the face which interacts with the complementary base on the other strand, e.g, at the 5′ position of a pyrimidine or a 7-position of a purine. These are discussed in more detail below;
(iii) nonphosphate linkages at the termini. Thus, preferred NRMs include Non-phosphate linkages, e.g., a linkage of 4 atoms which confers greater resistance to cleavage than does a phosphate bond. Examples include 3′ CH2-NCH₃—O—CH2-5′ and 3′ CH2-NH—(O═)—CH2-5′;
(iv) 3′-bridging thiophosphates and 5′-bridging thiophosphates. Thus, preferred NRM's can included these structures;
(v) L-RNA, 2′-5′ linkages, inverted linkages, a-nucleosides. Thus, other preferred NRM's include: L nucleosides and dimeric nucleotides derived from L-nucleosides; 2′-5′ phosphate, non-phosphate and modified phosphate linkages (e.g., thiophosphates, phosphoramidates and boronophosphates); dimers having inverted linkages, e.g., 3′-3′ or 5′-5′ linkages; monomers having an alpha linkage at the 1′ site on the sugar, e.g., the structures described herein having an alpha linkage;
(vi) conjugate groups. Thus, preferred NRM's can include e.g., a targeting moiety or a conjugated ligand described herein conjugated with the monomer, e.g., through the sugar, base, or backbone;
(vii) abasic linkages. Thus, preferred NRM's can include an abasic monomer, e.g., an abasic monomer as described herein (e.g., a nucleobaseless monomer); an aromatic or heterocyclic or polyheterocyclic aromatic monomer as described herein; and
(viii) 5′-phosphonates and 5′-phosphate prodrugs. Thus, preferred NRM's include monomers, preferably at the terminal position, e.g., the 5′ position, in which one or more atoms of the phosphate group is derivatized with a protecting group, which protecting group or groups, are removed as a result of the action of a component in the subject's body, e.g, a carboxyesterase or an enzyme present in the subject's body. E.g., a phosphate prodrug in which a carboxy esterase cleaves the protected molecule resulting in the production of a thioate anion which attacks a carbon adjacent to the 0 of a phosphate and resulting in the production of an unprotected phosphate.

One or more different NRM modifications can be introduced into an iRNA agent or into a sequence of an iRNA agent. An NRM modification can be used more than once in a sequence or in an iRNA agent. As some NRM's interfere with hybridization the total number incorporated, should be such that acceptable levels of iRNA agent duplex formation are maintained.

In some embodiments NRM modifications are introduced into the terminal the cleavage site or in the cleavage region of a sequence (a sense strand or sequence) which does not target a desired sequence or gene in the subject. This can reduce off-target silencing.

Evaluation of Candidate iRNA Agents

One can evaluate a candidate iRNA agent, e.g., a modified RNA, for a selected property by exposing the agent or modified molecule and a control molecule to the appropriate conditions and evaluating for the presence of the selected property. For example, resistance to a degradent can be evaluated as follows. A candidate modified RNA (and preferably a control molecule, usually the unmodified form) can be exposed to degradative conditions, e.g., exposed to a milieu, which includes a degradative agent, e.g., a nuclease. E.g., one can use a biological sample, e.g., one that is similar to a milieu, which might be encountered, in therapeutic use, e.g., blood or a cellular fraction, e.g., a cell-free homogenate or disrupted cells. The candidate and control could then be evaluated for resistance to degradation by any of a number of approaches. For example, the candidate and control could be labeled, preferably prior to exposure, with, e.g., a radioactive or enzymatic label, or a fluorescent label, such as Cy3 or Cy5. Control and modified RNA's can be incubated with the degradative agent, and optionally a control, e.g., an inactivated, e.g., heat inactivated, degradative agent. A physical parameter, e.g., size, of the modified and control molecules are then determined. They can be determined by a physical method, e.g., by polyacrylamide gel electrophoresis or a sizing column, to assess whether the molecule has maintained its original length, or assessed functionally. Alternatively, Northern blot analysis can be used to assay the length of an unlabeled modified molecule.

A functional assay can also be used to evaluate the candidate agent. A functional assay can be applied initially or after an earlier non-functional assay, (e.g., assay for resistance to degradation) to determine if the modification alters the ability of the molecule to silence gene expression. For example, a cell, e.g., a mammalian cell, such as a mouse or human cell, can be co-transfected with a plasmid expressing a fluorescent protein, e.g., GFP, and a candidate RNA agent homologous to the transcript encoding the fluorescent protein (see, e.g., WO 00/44914). For example, a modified dsRNA homologous to the GFP mRNA can be assayed for the ability to inhibit GFP expression by monitoring for a decrease in cell fluorescence, as compared to a control cell, in which the transfection did not include the candidate dsRNA, e.g., controls with no agent added and/or controls with a non-modified RNA added. Efficacy of the candidate agent on gene expression can be assessed by comparing cell fluorescence in the presence of the modified and unmodified dsRNA agents.

In an alternative functional assay, a candidate dsRNA agent homologous to an endogenous mouse gene, preferably a maternally expressed gene, such as c-mos, can be injected into an immature mouse oocyte to assess the ability of the agent to inhibit gene expression in vivo (see, e.g., WO 01/36646). A phenotype of the oocyte, e.g., the ability to maintain arrest in metaphase II, can be monitored as an indicator that the agent is inhibiting expression. For example, cleavage of c-mos mRNA by a dsRNA agent would cause the oocyte to exit metaphase arrest and initiate parthenogenetic development (Colledge et al. Nature 370: 65-68, 1994; Hashimoto et al. Nature, 370:68-71, 1994). The effect of the modified agent on target RNA levels can be verified by Northern blot to assay for a decrease in the level of target mRNA, or by Western blot to assay for a decrease in the level of target protein, as compared to a negative control. Controls can include cells in which with no agent is added and/or cells in which a non-modified RNA is added.

### REFERENCES

### General References

The oligoribonucleotides and oligoribonucleosides used in accordance with this invention may be synthesized with solid phase synthesis, see for example “Oligonucleotide synthesis, a practical approach”, Ed. M. J. Gait, IRL Press, 1984; “Oligonucleotides and Analogues, A Practical Approach”, Ed. F. Eckstein, IRL Press, 1991 (especially Chapter 1, Modern machine-aided methods of oligodeoxyribonucleotide synthesis, Chapter 2, Oligoribonucleotide synthesis, Chapter 3,2′-O-Methyloligoribonucleotides: synthesis and applications, Chapter 4, Phosphorothioate oligonucleotides, Chapter 5, Synthesis of oligonucleotide phosphorodithioates, Chapter 6, Synthesis of oligo-2′-deoxyribonucleoside methylphosphonates, and. Chapter 7, Oligodeoxynucleotides containing modified bases. Other particularly useful synthetic procedures, reagents, blocking groups and reaction conditions are described in Martin, P., Helv. Chim. Acta, 1995, 78, 486-504; Beaucage, S. L. and Iyer, R. P., Tetrahedron, 1992, 48, 2223-2311 and Beaucage, S. L. and Iyer, R. P., Tetrahedron, 1993, 49, 6123-6194, or references referred to therein.

Modification described in WO 00/44895, WO01/75164, or WO02/44321 can be used herein.

The disclosure of all publications, patents, and published patent applications listed herein are hereby incorporated by reference.

### Phosphate Group References

The preparation of phosphinate oligoribonucleotides is described in U.S. Pat. No. 5,508,270. The preparation of alkyl phosphonate oligoribonucleotides is described in U.S. Pat. No. 4,469,863. The preparation of phosphoramidite oligoribonucleotides is described in U.S. Pat. No. 5,256,775 or U.S. Pat. No. 5,366,878. The preparation of phosphotriester oligoribonucleotides is described in U.S. Pat. No. 5,023,243. The preparation of borano phosphate oligoribonucleotide is described in U.S. Pat. Nos. 5,130,302 and 5,177,198. The preparation of 3′-Deoxy-3′-amino phosphoramidate oligoribonucleotides is described in U.S. Pat. No. 5,476,925. 3′-Deoxy-3′-methylenephosphonate oligoribonucleotides is described in An, H, et al. J. Org. Chem. 2001, 66, 2789-2801. Preparation of sulfur bridged nucleotides is described in Sproat et al. Nucleosides Nucleotides 1988, 7,651 and Crosstick et al. Tetrahedron Lett. 1989, 30, 4693.

### Sugar Group References

Modifications to the 2′ modifications can be found in Verma, S. et al. Annu. Rev. Biochem. 1998, 67, 99-134 and all references therein. Specific modifications to the ribose can be found in the following references: 2′-fluoro (Kawasaki et. al., J. Med. Chem., 1993, 36, 831-841), 2′-MOE (Martin, P. Helv. Chim. Acta 1996, 79, 1930-1938), “LNA” (Wengel, J. Acc. Chem. Res. 1999, 32, 301-310).

### Replacement of the Phosphate Group References

Methylenemethylimino linked oligoribonucleosides, also identified herein as MMI linked oligoribonucleosides, methylenedimethylhydrazo linked oligoribonucleosides, also identified herein as MDH linked oligoribonucleosides, and methylenecarbonylamino linked oligonucleosides, also identified herein as amide-3 linked oligoribonucleosides, and methyleneaminocarbonyl linked oligonucleosides, also identified herein as amide-4 linked oligoribonucleosides as well as mixed backbone compounds having, as for instance, alternating MMI and PO or PS linkages can be prepared as is described in U.S. Pat. Nos. 5,378,825, 5,386,023, 5,489,677 and in published PCT applications PCT/US92/04294 and PCT/US92/04305 (published as WO 92/20822 WO and 92/20823, respectively). Formacetal and thioformacetal linked oligoribonucleosides can be prepared as is described in U.S. Pat. Nos. 5,264,562 and 5,264,564. Ethylene oxide linked oligoribonucleosides can be prepared as is described in U.S. Pat. No. 5,223,618. Siloxane replacements are described in Cormier, J. F. et al. Nucleic Acids Res. 1988, 16, 4583. Carbonate replacements are described in Tittensor, J. R. J. Chem. Soc. C 1971, 1933. Carboxymethyl replacements are described in Edge, M. D. et al. J. Chem. Soc. Perkin Trans. 11972, 1991. Carbamate replacements are described in Stirchak, E. P. Nucleic Acids Res. 1989, 17, 6129.

### Replacement of the Phosphate-Ribose Backbone References

Cyclobutyl sugar surrogate compounds can be prepared as is described in U.S. Pat. No. 5,359,044. Pyrrolidine sugar surrogate can be prepared as is described in U.S. Pat. No. 5,519,134. Morpholino sugar surrogates can be prepared as is described in U.S. Pat. Nos. 5,142,047 and 5,235,033, and other related patent disclosures. Peptide Nucleic Acids (PNAs) are known per se and can be prepared in accordance with any of the various procedures referred to in Peptide Nucleic Acids (PNA): Synthesis, Properties and Potential Applications, Bioorganic & Medicinal Chemistry, 1996, 4, 5-23. They may also be prepared in accordance with U.S. Pat. No. 5,539,083.

### Terminal Modification References

Terminal modifications are described in Manoharan, M. et al. Antisense and Nucleic Acid Drug Development 12, 103-128 (2002) and references therein.

### Bases References

N-2 substituted purine nucleoside amidites can be prepared as is described in U.S. Pat. No. 5,459,255. 3-Deaza purine nucleoside amidites can be prepared as is described in U.S. Pat. No. 5,457,191. 5,6-Substituted pyrimidine nucleoside amidites can be prepared as is described in U.S. Pat. No. 5,614,617. 5-Propynyl pyrimidine nucleoside amidites can be prepared as is described in U.S. Pat. No. 5,484,908. Additional references are disclosed in the above section on base modifications.

### Oligonucleotide Production

The oligonucleotide compounds of the invention can be prepared using solution-phase or solid-phase organic synthesis. Organic synthesis offers the advantage that the oligonucleotide strands comprising non-natural or modified nucleotides can be easily prepared. Any other means for such synthesis known in the art may additionally or alternatively be employed. It is also known to use similar techniques to prepare other oligonucleotides, such as the phosphorothioates, phosphorodithioates and alkylated derivatives. The double-stranded oligonucleotide compounds of the invention comprising non-natural nucleobases and optionally non-natural sugar moieties may be prepared using a two-step procedure. First, the individual strands of the double-stranded molecule are prepared separately. Then, the component strands are annealed.

Regardless of the method of synthesis, the iRNA preparation can be prepared in a solution (e.g., an aqueous and/or organic solution) that is appropriate for formulation. For example, the iRNA preparation can be precipitated and redissolved in pure double-distilled water, and lyophilized. The dried iRNA can then be resuspended in a solution appropriate for the intended formulation process.

Teachings regarding the synthesis of particular modified oligonucleotides may be found in the following U.S. patents or pending patent applications: U.S. Pat. Nos. 5,138,045 and 5,218,105, drawn to polyamine conjugated oligonucleotides; U.S. Pat. No. 5,212,295, drawn to monomers for the preparation of oligonucleotides having chiral phosphorus linkages; U.S. Pat. Nos. 5,378,825 and 5,541,307, drawn to oligonucleotides having modified backbones; U.S. Pat. No. 5,386,023, drawn to backbone-modified oligonucleotides and the preparation thereof through reductive coupling; U.S. Pat. No. 5,457,191, drawn to modified nucleobases based on the 3-deazapurine ring system and methods of synthesis thereof; U.S. Pat. No. 5,459,255, drawn to modified nucleobases based on N-2 substituted purines; U.S. Pat. No. 5,521,302, drawn to processes for preparing oligonucleotides having chiral phosphorus linkages; U.S. Pat. No. 5,539,082, drawn to peptide nucleic acids; U.S. Pat. No. 5,554,746, drawn to oligonucleotides having .beta.-lactam backbones; U.S. Pat. No. 5,571,902, drawn to methods and materials for the synthesis of oligonucleotides; U.S. Pat. No. 5,578,718, drawn to nucleosides having alkylthio groups, wherein such groups may be used as linkers to other moieties attached at any of a variety of positions of the nucleoside; U.S. Pat. Nos. 5,587,361 and 5,599,797, drawn to oligonucleotides having phosphorothioate linkages of high chiral purity; U.S. Pat. No. 5,506,351, drawn to processes for the preparation of 2′-O-alkyl guanosine and related compounds, including 2,6-diaminopurine compounds; U.S. Pat. No. 5,587,469, drawn to oligonucleotides having N-2 substituted purines; U.S. Pat. No. 5,587,470, drawn to oligonucleotides having 3-deazapurines; U.S. Pat. No. 5,223,168, and U.S. Pat. No. 5,608,046, both drawn to conjugated 4′-desmethyl nucleoside analogs; U.S. Pat. Nos. 5,602,240, and 5,610,289, drawn to backbone-modified oligonucleotide analogs; and U.S. Pat. Nos. 6,262,241, and 5,459,255, drawn to, inter alia, methods of synthesizing 2′-fluoro-oligonucleotides.

It is not necessary for all positions in a given compound to be uniformly modified, and in fact more than one type of modification may be incorporated in a single oligonucleotide compound or even in a single nucleotide thereof.

### Routes of Delivery

For ease of exposition the formulations, compositions and methods in this section are discussed largely with regard to unmodified iRNA agents. It should be understood, however, that these formulations, compositions and methods can be practiced with other iRNA agents, e.g., modified iRNA agents, and such practice is within the invention. A composition that includes an iRNA can be delivered to a subject by a variety of routes. Exemplary routes include: intravenous, topical, rectal, anal, vaginal, nasal, pulmonary, ocular.

The iRNA molecules of the invention can be incorporated into pharmaceutical compositions suitable for administration. Such compositions typically include one or more species of iRNA and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

The pharmaceutical compositions of the present invention may be administered in a number of ways depending upon whether local or systemic treatment is desired and upon the area to be treated. Administration may be topical (including ophthalmic, vaginal, rectal, intranasal, transdermal), oral or parenteral. Parenteral administration includes intravenous drip, subcutaneous, intraperitoneal or intramuscular injection, or intrathecal or intraventricular administration.

The route and site of administration may be chosen to enhance targeting. For example, to target muscle cells, intramuscular injection into the muscles of interest would be a logical choice. Lung cells might be targeted by administering the iRNA in aerosol form. The vascular endothelial cells could be targeted by coating a balloon catheter with the iRNA and mechanically introducing the DNA.

Formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable. Coated condoms, gloves and the like may also be useful.

Compositions for oral administration include powders or granules, suspensions or solutions in water, syrups, elixirs or non-aqueous media, tablets, capsules, lozenges, or troches. In the case of tablets, carriers that can be used include lactose, sodium citrate and salts of phosphoric acid. Various disintegrants such as starch, and lubricating agents such as magnesium stearate, sodium lauryl sulfate and talc, are commonly used in tablets. For oral administration in capsule form, useful diluents are lactose and high molecular weight polyethylene glycols. When aqueous suspensions are required for oral use, the nucleic acid compositions can be combined with emulsifying and suspending agents. If desired, certain sweetening and/or flavoring agents can be added.

Compositions for intrathecal or intraventricular administration may include sterile aqueous solutions which may also contain buffers, diluents and other suitable additives.

Formulations for parenteral administration may include sterile aqueous solutions which may also contain buffers, diluents and other suitable additives. Intraventricular injection may be facilitated by an intraventricular catheter, for example, attached to a reservoir. For intravenous use, the total concentration of solutes should be controlled to render the preparation isotonic.

For ocular administration, ointments or droppable liquids may be delivered by ocular delivery systems known to the art such as applicators or eye droppers. Such compositions can include mucomimetics such as hyaluronic acid, chondroitin sulfate, hydroxypropyl methylcellulose or poly(vinyl alcohol), preservatives such as sorbic acid, EDTA or benzylchronium chloride, and the usual quantities of diluents and/or carriers.

### Topical Delivery

For ease of exposition the formulations, compositions and methods in this section are discussed largely with regard to unmodified iRNA agents. It should be understood, however, that these formulations, compositions and methods can be practiced with other iRNA agents, e.g., modified iRNA agents, and such practice is within the invention. In a preferred embodiment, an iRNA agent is delivered to a subject via topical administration. “Topical administration” refers to the delivery to a subject by contacting the formulation directly to a surface of the subject. The most common form of topical delivery is to the skin, but a composition disclosed herein can also be directly applied to other surfaces of the body, e.g., to the eye, a mucous membrane, to surfaces of a body cavity or to an internal surface. As mentioned above, the most common topical delivery is to the skin. The term encompasses several routes of administration including, but not limited to, topical and transdermal. These modes of administration typically include penetration of the skin's permeability barrier and efficient delivery to the target tissue or stratum. Topical administration can be used as a means to penetrate the epidermis and dermis and ultimately achieve systemic delivery of the composition. Topical administration can also be used as a means to selectively deliver oligonucleotides to the epidermis or dermis of a subject, or to specific strata thereof; or to an underlying tissue.

The term “skin,” as used herein, refers to the epidermis and/or dermis of an animal. Mammalian skin consists of two major, distinct layers. The outer layer of the skin is called the epidermis. The epidermis is comprised of the stratum corneum, the stratum granulosum, the stratum spinosum, and the stratum basale, with the stratum corneum being at the surface of the skin and the stratum basale being the deepest portion of the epidermis. The epidermis is between 50 μm and 0.2 mm thick, depending on its location on the body.

Beneath the epidermis is the dermis, which is significantly thicker than the epidermis. The dermis is primarily composed of collagen in the form of fibrous bundles. The collagenous bundles provide support for, inter alia, blood vessels, lymph capillaries, glands, nerve endings and immunologically active cells.

One of the major functions of the skin as an organ is to regulate the entry of substances into the body. The principal permeability barrier of the skin is provided by the stratum corneum, which is formed from many layers of cells in various states of differentiation. The spaces between cells in the stratum corneum is filled with different lipids arranged in lattice-like formations that provide seals to further enhance the skins permeability barrier.

The permeability barrier provided by the skin is such that it is largely impermeable to molecules having molecular weight greater than about 750 Da. For larger molecules to cross the skin's permeability barrier, mechanisms other than normal osmosis must be used.

Several factors determine the permeability of the skin to administered agents. These factors include the characteristics of the treated skin, the characteristics of the delivery agent, interactions between both the drug and delivery agent and the drug and skin, the dosage of the drug applied, the form of treatment, and the post treatment regimen. To selectively target the epidermis and dermis, it is sometimes possible to formulate a composition that comprises one or more penetration enhancers that will enable penetration of the drug to a preselected stratum.

Transdermal delivery is a valuable route for the administration of lipid soluble therapeutics. The dermis is more permeable than the epidermis and therefore absorption is much more rapid through abraded, burned or denuded skin. Inflammation and other physiologic conditions that increase blood flow to the skin also enhance transdermal adsorption. Absorption via this route may be enhanced by the use of an oily vehicle (inunction) or through the use of one or more penetration enhancers. Other effective ways to deliver a composition disclosed herein via the transdermal route include hydration of the skin and the use of controlled release topical patches. The transdermal route provides a potentially effective means to deliver a composition disclosed herein for systemic and/or local therapy.

In addition, iontophoresis (transfer of ionic solutes through biological membranes under the influence of an electric field) (Lee et al., Critical Reviews in Therapeutic Drug Carrier Systems, 1991, p. 163), phonophoresis or sonophoresis (use of ultrasound to enhance the absorption of various therapeutic agents across biological membranes, notably the skin and the cornea) (Lee et al., Critical Reviews in Therapeutic Drug Carrier Systems, 1991, p. 166), and optimization of vehicle characteristics relative to dose position and retention at the site of administration (Lee et al., Critical Reviews in Therapeutic Drug Carrier Systems, 1991, p. 168) may be useful methods for enhancing the transport of topically applied compositions across skin and mucosal sites.

The compositions and methods provided may also be used to examine the function of various proteins and genes in vitro in cultured or preserved dermal tissues and in animals. The invention can be thus applied to examine the function of any gene. The methods of the invention can also be used therapeutically or prophylactically. For example, for the treatment of animals that are known or suspected to suffer from diseases such as psoriasis, lichen planus, toxic epidermal necrolysis, ertythema multiforme, basal cell carcinoma, squamous cell carcinoma, malignant melanoma, Paget's disease, Kaposi's sarcoma, pulmonary fibrosis, Lyme disease and viral, fungal and bacterial infections of the skin.

### Pulmonary Delivery

For ease of exposition the formulations, compositions and methods in this section are discussed largely with regard to unmodified iRNA agents. It should be understood, however, that these formulations, compositions and methods can be practiced with other iRNA agents, e.g., modified iRNA agents, and such practice is within the invention. A composition that includes an iRNA agent, e.g., a double-stranded iRNA agent, can be administered to a subject by pulmonary delivery. Pulmonary delivery compositions can be delivered by inhalation by the patient of a dispersion so that the composition, preferably iRNA, within the dispersion can reach the lung where it can be readily absorbed through the alveolar region directly into blood circulation. Pulmonary delivery can be effective both for systemic delivery and for localized delivery to treat diseases of the lungs.

Pulmonary delivery can be achieved by different approaches, including the use of nebulized, aerosolized, micellular and dry powder-based formulations. Delivery can be achieved with liquid nebulizers, aerosol-based inhalers, and dry powder dispersion devices. Metered-dose devices are preferred. One of the benefits of using an atomizer or inhaler is that the potential for contamination is minimized because the devices are self contained. Dry powder dispersion devices, for example, deliver drugs that may be readily formulated as dry powders. An iRNA composition may be stably stored as lyophilized or spray-dried powders by itself or in combination with suitable powder carriers. The delivery of a composition for inhalation can be mediated by a dosing timing element which can include a timer, a dose counter, time measuring device, or a time indicator which when incorporated into the device enables dose tracking, compliance monitoring, and/or dose triggering to a patient during administration of the aerosol medicament.

The term “powder” means a composition that consists of finely dispersed solid particles that are free flowing and capable of being readily dispersed in an inhalation device and subsequently inhaled by a subject so that the particles reach the lungs to permit penetration into the alveoli. Thus, the powder is said to be “respirable.” Preferably the average particle size is less than about 10 μm in diameter preferably with a relatively uniform spheroidal shape distribution. More preferably the diameter is less than about 7.5 μm and most preferably less than about 5.0 μm. Usually the particle size distribution is between about 0.1 μm and about 5 μm in diameter, particularly about 0.3 μm to about 5 μm.

The term “dry” means that the composition has a moisture content below about 10% by weight (% w) water, usually below about 5% w and preferably less it than about 3% w. A dry composition can be such that the particles are readily dispersible in an inhalation device to form an aerosol.

The term “therapeutically effective amount” is the amount present in the composition that is needed to provide the desired level of drug in the subject to be treated to give the anticipated physiological response.

The term “physiologically effective amount” is that amount delivered to a subject to give the desired palliative or curative effect.

The term “pharmaceutically acceptable carrier” means that the carrier can be taken into the lungs with no significant adverse toxicological effects on the lungs.

The types of pharmaceutical excipients that are useful as carrier include stabilizers such as human serum albumin (HSA), bulking agents such as carbohydrates, amino acids and polypeptides; pH adjusters or buffers; salts such as sodium chloride; and the like. These carriers may be in a crystalline or amorphous form or may be a mixture of the two.

Bulking agents that are particularly valuable include compatible carbohydrates, polypeptides, amino acids or combinations thereof. Suitable carbohydrates include monosaccharides such as galactose, D-mannose, sorbose, and the like; disaccharides, such as lactose, trehalose, and the like; cyclodextrins, such as 2-hydroxypropyl-.beta.-cyclodextrin; and polysaccharides, such as raffinose, maltodextrins, dextrans, and the like; alditols, such as mannitol, xylitol, and the like. A preferred group of carbohydrates includes lactose, threhalose, raffinose maltodextrins, and mannitol. Suitable polypeptides include aspartame. Amino acids include alanine and glycine, with glycine being preferred.

Additives, which are minor components of the composition of this invention, may be included for conformational stability during spray drying and for improving dispersibility of the powder. These additives include hydrophobic amino acids such as tryptophan, tyrosine, leucine, phenylalanine, and the like.

Suitable pH adjusters or buffers include organic salts prepared from organic acids and bases, such as sodium citrate, sodium ascorbate, and the like; sodium citrate is preferred.

Pulmonary administration of a micellar iRNA formulation may be achieved through metered dose spray devices with propellants such as tetrafluoroethane, heptafluoroethane, dimethylfluoropropane, tetrafluoropropane, butane, isobutane, dimethyl ether and other non-CFC and CFC propellants.

### Oral or Nasal Delivery

For ease of exposition the formulations, compositions and methods in this section are discussed largely with regard to unmodified iRNA agents. It should be understood, however, that these formulations, compositions and methods can be practiced with other iRNA agents, e.g., modified iRNA agents, and such practice is within the invention. Both the oral and nasal membranes offer advantages over other routes of administration. For example, drugs administered through these membranes have a rapid onset of action, provide therapeutic plasma levels, avoid first pass effect of hepatic metabolism, and avoid exposure of the drug to the hostile gastrointestinal (GI) environment. Additional advantages include easy access to the membrane sites so that the drug can be applied, localized and removed easily.

In oral delivery, compositions can be targeted to a surface of the oral cavity, e.g., to sublingual mucosa which includes the membrane of ventral surface of the tongue and the floor of the mouth or the buccal mucosa which constitutes the lining of the cheek. The sublingual mucosa is relatively permeable thus giving rapid absorption and acceptable bioavailability of many drugs. Further, the sublingual mucosa is convenient, acceptable and easily accessible.

The ability of molecules to permeate through the oral mucosa appears to be related to molecular size, lipid solubility and peptide protein ionization. Small molecules, less than 1000 daltons appear to cross mucosa rapidly. As molecular size increases, the permeability decreases rapidly. Lipid soluble compounds are more permeable than non-lipid soluble molecules. Maximum absorption occurs when molecules are un-ionized or neutral in electrical charges. Therefore charged molecules present the biggest challenges to absorption through the oral mucosae.

A pharmaceutical composition of iRNA may also be administered to the buccal cavity of a human being by spraying into the cavity, without inhalation, from a metered dose spray dispenser, a mixed micellar pharmaceutical formulation as described above and a propellant. In one embodiment, the dispenser is first shaken prior to spraying the pharmaceutical formulation and propellant into the buccal cavity.

### Devices

For ease of exposition the devices, formulations, compositions and methods in this section are discussed largely with regard to unmodified iRNA agents. It should be understood, however, that these devices, formulations, compositions and methods can be practiced with other iRNA agents, e.g., modified iRNA agents, and such practice is within the invention. An iRNA agent, e.g., a double-stranded iRNA agent, or sRNA agent, (e.g., a precursor, e.g., a larger iRNA agent which can be processed into a sRNA agent, or a DNA which encodes an iRNA agent, e.g., a double-stranded iRNA agent, or sRNA agent, or precursor thereof) can be disposed on or in a device, e.g., a device which implanted or otherwise placed in a subject. Exemplary devices include devices which are introduced into the vasculature, e.g., devices inserted into the lumen of a vascular tissue, or which devices themselves form a part of the vasculature, including stents, catheters, heart valves, and other vascular devices. These devices, e.g., catheters or stents, can be placed in the vasculature of the lung, heart, or leg.

Other devices include non-vascular devices, e.g., devices implanted in the peritoneum, or in organ or glandular tissue, e.g., artificial organs. The device can release a therapeutic substance in addition to a iRNA, e.g., a device can release insulin.

Other devices include artificial joints, e.g., hip joints, and other orthopedic implants.

In one embodiment, unit doses or measured doses of a composition that includes iRNA are dispensed by an implanted device. The device can include a sensor that monitors a parameter within a subject. For example, the device can include pump, e.g., and, optionally, associated electronics.

Tissue, e.g., cells or organs, such as the kidney, can be treated with an iRNA agent ex vivo and then administered or implanted in a subject.

The tissue can be autologous, allogeneic, or xenogeneic tissue. For example, tissue (e.g., kidney) can be treated to reduce graft v. host disease. In other embodiments, the tissue is allogeneic and the tissue is treated to treat a disorder characterized by unwanted gene expression in that tissue, such as in the kidney. In another example, tissue containing hematopoietic cells, e.g., bone marrow hematopoietic cells, can be treated to inhibit unwanted cell proliferation.

Introduction of treated tissue, whether autologous or transplant, can be combined with other therapies.

In some implementations, the iRNA treated cells are insulated from other cells, e.g., by a semi-permeable porous barrier that prevents the cells from leaving the implant, but enables molecules from the body to reach the cells and molecules produced by the cells to enter the body. In one embodiment, the porous barrier is formed from alginate.

In one embodiment, a contraceptive device is coated with or contains an iRNA agent. Exemplary devices include condoms, diaphragms, IUD (implantable uterine devices, sponges, vaginal sheaths, and birth control devices. In one embodiment, the iRNA is chosen to inactive sperm or egg. In another embodiment, the iRNA is chosen to be complementary to a viral or pathogen RNA, e.g., an RNA of an STD. In some instances, the iRNA composition can include a spermicidal agent.

### Formulations

The iRNA agents described herein can be formulated for administration to a subject. For ease of exposition the formulations, compositions and methods in this section are discussed largely with regard to unmodified iRNA agents. It should be understood, however, that these formulations, compositions and methods can be practiced with other iRNA agents, e.g., modified iRNA agents, and such practice is within the invention.

A formulated iRNA composition can assume a variety of states. In some examples, the composition is at least partially crystalline, uniformly crystalline, and/or anhydrous (e.g., less than 80, 50, 30, 20, or 10% water). In another example, the iRNA is in an aqueous phase, e.g., in a solution that includes water.

The aqueous phase or the crystalline compositions can, e.g., be incorporated into a delivery vehicle, e.g., a liposome (particularly for the aqueous phase) or a particle (e.g., a microparticle as can be appropriate for a crystalline composition). Generally, the iRNA composition is formulated in a manner that is compatible with the intended method of administration.

In particular embodiments, the composition is prepared by at least one of the following methods: spray drying, lyophilization, vacuum drying, evaporation, fluid bed drying, or a combination of these techniques; or sonication with a lipid, freeze-drying, condensation and other self-assembly.

An iRNA preparation can be formulated in combination with another agent, e.g., another therapeutic agent or an agent that stabilizes a iRNA, e.g., a protein that complexes with iRNA to form an iRNP. Still other agents include chelators, e.g., EDTA (e.g., to remove divalent cations such as Mg²⁺), salts, RNAse inhibitors (e.g., a broad specificity RNAse inhibitor such as RNAsin) and so forth.

In one embodiment, the mRNA preparation includes another iRNA agent, e.g., a second iRNA that can mediated RNAi with respect to a second gene, or with respect to the same gene. Still other preparation can include at least 3, 5, ten, twenty, fifty, or a hundred or more different iRNA species. Such iRNAs can mediated RNAi with respect to a similar number of different genes.

In one embodiment, the iRNA preparation includes at least a second therapeutic agent (e.g., an agent other than a RNA or a DNA). For example, an iRNA composition for the treatment of a viral disease, e.g. HIV, might include a known antiviral agent (e.g., a protease inhibitor or reverse transcriptase inhibitor). In another example, an iRNA composition for the treatment of a cancer might further comprise a chemotherapeutic agent.

### Pharmaceutical Compositions

In one embodiment, the invention relates to a pharmaceutical composition containing a modified iRNA agent, as described in the preceding sections, and a pharmaceutically acceptable carrier, as described below. A pharmaceutical composition including the modified iRNA agent is useful for treating a disease caused by expression of a target gene. In this aspect of the invention, the iRNA agent of the invention is formulated as described below. The pharmaceutical composition is administered in a dosage sufficient to inhibit expression of the target gene.

The pharmaceutical compositions of the present invention are administered in dosages sufficient to inhibit the expression or activity of the target gene. Compositions containing the iRNA agent of the invention can be administered at surprisingly low dosages. A maximum dosage of 5 mg iRNA agent per kilogram body weight per day may be sufficient to inhibit or completely suppress the expression or activity of the target gene.

In general, a suitable dose of modified iRNA agent will be in the range of 0.001 to 500 milligrams per kilogram body weight of the recipient per day (e.g., about 1 microgram per kilogram to about 500 milligrams per kilogram, about 100 micrograms per kilogram to about 100 milligrams per kilogram, about 1 milligrams per kilogram to about 75 milligrams per kilogram, about 10 micrograms per kilogram to about 50 milligrams per kilogram, or about 1 microgram per kilogram to about 50 micrograms per kilogram). The pharmaceutical composition may be administered once per day, or the iRNA agent may be administered as two, three, four, five, six or more sub-doses at appropriate intervals throughout the day. In that case, the iRNA agent contained in each sub-dose must be correspondingly smaller in order to achieve the total daily dosage. The dosage unit can also be compounded for delivery over several days, e.g., using a conventional sustained release formulation which provides sustained release of the iRNA agent over a several day period. Sustained release formulations are well known in the art. In this embodiment, the dosage unit contains a corresponding multiple of the daily dose.

The skilled artisan will appreciate that certain factors may influence the dosage and timing required to effectively treat a subject, including but not limited to the severity of the infection or disease, previous treatments, the general health and/or age of the subject, and other diseases present. Moreover, treatment of a subject with a therapeutically effective amount of a composition can include a single treatment or a series of treatments. Estimates of effective dosages and in vivo half-lives for the individual iRNA agent encompassed by the invention can be made using conventional methodologies or on the basis of in vivo testing using an appropriate animal model, as described elsewhere herein.

Advances in mouse genetics have generated a number of mouse models for the study of various human diseases. For example, mouse repositories can be found at The Jackson Laboratory, Charles River Laboratories, Taconic, Harlan, Mutant Mouse Regional Resource Centers (MMRRC) National Network and at the European Mouse Mutant Archive. Such models may be used for in vivo testing of iRNA agent, as well as for determining a therapeutically effective dose.

The pharmaceutical compositions encompassed by the invention may be administered by any means known in the art including, but not limited to oral or parenteral routes, including intravenous, intramuscular, intraperitoneal, subcutaneous, transdermal, airway (aerosol), ocular, rectal, vaginal and topical (including buccal and sublingual) administration. In preferred embodiments, the pharmaceutical compositions are administered by intravenous or intraparenteral infusion or injection. The pharmaceutical compositions can also be administered intraparenchymally, intrathecally, and/or by stereotactic injection.

For oral administration, the iRNA agent useful in the invention will generally be provided in the form of tablets or capsules, as a powder or granules, or as an aqueous solution or suspension.

Tablets for oral use may include the active ingredients mixed with pharmaceutically acceptable excipients such as inert diluents, disintegrating agents, binding agents, lubricating agents, sweetening agents, flavoring agents, coloring agents and preservatives. Suitable inert diluents include sodium and calcium carbonate, sodium and calcium phosphate, and lactose, while corn starch and alginic acid are suitable disintegrating agents. Binding agents may include starch and gelatin, while the lubricating agent, if present, will generally be magnesium stearate, stearic acid or talc. If desired, the tablets may be coated with a material such as glyceryl monostearate or glyceryl distearate, to delay absorption in the gastrointestinal tract.

Capsules for oral use include hard gelatin capsules in which the active ingredient is mixed with a solid diluent, and soft gelatin capsules wherein the active ingredient is mixed with water or an oil such as peanut oil, liquid paraffin or olive oil.

For intramuscular, intraperitoneal, subcutaneous and intravenous use, the pharmaceutical compositions of the invention will generally be provided in sterile aqueous solutions or suspensions, buffered to an appropriate pH and isotonicity. Suitable aqueous vehicles include Ringer's solution and isotonic sodium chloride. In a preferred embodiment, the carrier consists exclusively of an aqueous buffer. In this context, “exclusively” means no auxiliary agents or encapsulating substances are present which might affect or mediate uptake of iRNA agent in the cells that harbor the target gene or virus. Such substances include, for example, micellar structures, such as liposomes or capsids, as described below. Although microinjection, lipofection, viruses, viroids, capsids, capsoids, or other auxiliary agents are required to introduce iRNA agent into cell cultures, surprisingly these methods and agents are not necessary for uptake of iRNA agent in vivo. The iRNA agent of the present invention are particularly advantageous in that they do not require the use of an auxiliary agent to mediate uptake of the iRNA agent into the cell, many of which agents are toxic or associated with deleterious side effects. Aqueous suspensions according to the invention may include suspending agents such as cellulose derivatives, sodium alginate, polyvinyl-pyrrolidone and gum tragacanth, and a wetting agent such as lecithin. Suitable preservatives for aqueous suspensions include ethyl and n-propyl p-hydroxybenzoate.

The pharmaceutical compositions can also include encapsulated formulations to protect the iRNA agent against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811; PCT publication WO 91/06309; and European patent publication EP-A-43075, which are incorporated by reference herein.

Toxicity and therapeutic efficacy of iRNA agent can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. iRNA agents that exhibit high therapeutic indices are preferred.

The data obtained from cell culture assays and animal studies can be used in formulating a range of dosage for use in humans. The dosages of compositions of the invention are preferably within a range of circulating concentrations that include the ED50 with little or no toxicity. The dosage may vary within this range depending upon the dosage form employed and the route of administration utilized. For any iRNA agent used in the method of the invention, the therapeutically effective dose can be estimated initially from cell culture assays. A dose may be formulated in animal models to achieve a circulating plasma concentration range of the iRNA agent or, when appropriate, of the polypeptide product of a target sequence (e.g., achieving a decreased concentration of the polypeptide) that includes the IC50 (i.e., the concentration of the test iRNA agent which achieves a half-maximal inhibition of symptoms) as determined in cell culture. Such information can be used to more accurately determine useful doses in humans. Levels in plasma may be measured, for example, by high performance liquid chromatography.

In addition to their administration individually or as a plurality, as discussed above, iRNA agents relating to the invention can be administered in combination with other known agents effective in treating viral infections and diseases. In any event, the administering physician can adjust the amount and timing of iRNA agent administration on the basis of results observed using standard measures of efficacy known in the art or described herein.

### Methods for Inhibiting Expression of a Target Gene

In yet another aspect, the invention relates to a method for inhibiting the expression of a target gene in a cell or organism. In one embodiment, the method includes administering the inventive iRNA agent or a pharmaceutical composition containing the iRNA agent to a cell or an organism, such as a mammal, such that expression of the target gene is silenced. Because of their surprisingly improved stability and bioavailability, the iRNA agent of the present invention effectively inhibit expression or activity of target genes at surprisingly low dosages. Compositions and methods for inhibiting the expression of a target gene using iRNA agent can be performed as described in the preceding sections, particularly Sections 4 and 5.

In this embodiment, a pharmaceutical composition containing the iRNA agent may be administered by any means known in the art including, but not limited to oral or parenteral routes, including intravenous, intramuscular, intraperitoneal, subcutaneous, transdermal, airway (aerosol), ocular, rectal, vaginal, and topical (including buccal and sublingual) administration. In preferred embodiments, the pharmaceutical compositions are administered by intravenous or intraparenteral infusion or injection. The pharmaceutical compositions can also be administered intraparenchymally, intrathecally, and/or by stereotactic injection.

The methods for inhibiting the expression of a target gene can be applied to any gene one wishes to silence, thereby specifically inhibiting its expression, provided the cell or organism in which the target gene is expressed includes the cellular machinery which effects RNA interference. Examples of genes which can be targeted for silencing include, without limitation, developmental genes including but not limited to adhesion molecules, cyclin kinase inhibitors, Wnt family members, Pax family members, Winged helix family members, Hox family members, cytokines/lymphokines and their receptors, growth/differentiation factors and their receptors, and neurotransmitters and their receptors; (2) oncogenes including but not limited to ABLI, BCL1, BCL2, BCL6, CBFA2, CBL, CSFIR, ERBA, ERBB, EBRB2, ETS1, ETS1, ETV6, FGR, FOS, FYN, HCR, HRAS, JUN, KRAS, LCK, LYN, MDM2, MLL, MYB, MYC, MYCL1, MYCN, NRAS, PIM1, PML, RET, SRC, TAL1, TCL3 and YES; (3) tumor suppresser genes including but not limited to APC, BRCA1, BRCA2, MADH4, MCC, NF1, NF2, RB1, TP53 and WT1; and (4) enzymes including but not limited to ACP desaturases and hydroxylases, ADP-glucose pyrophorylases, ATPases, alcohol dehydrogenases, amylases, amyloglucosidases, catalases, cellulases, cyclooxygenases, decarboxylases, dextrinases, DNA and RNA polymerases, galactosidases, glucanases, glucose oxidases, GTPases, helicases, hemicellulases, integrases, invertases, isomerases, kinases, lactases, lipases, lipoxygenases, lysozymes, pectinesterases, peroxidases, phosphatases, phospholipases, phosphorylases, polygalacturonases, proteinases and peptideases, pullanases, recombinases, reverse transcriptases, topoisomerases, and xylanases.

In addition to in vivo gene inhibition, the skilled artisan will appreciate that the iRNA agent of the present invention are useful in a wide variety of in vitro applications. Such in vitro applications, include, for example, scientific and commercial research (e.g., elucidation of physiological pathways, drug discovery and development), and medical and veterinary diagnostics. In general, the method involves the introduction of the iRNA agent into a cell using known techniques (e.g., absorption through cellular processes, or by auxiliary agents or devices, such as electroporation and lipofection), then maintaining the cell for a time sufficient to obtain degradation of an mRNA transcript of the target gene.

### Methods for Treating Diseases Caused by Expression of a Target Gene

In one embodiment, the invention relates to a method for treating a subject having a disease or at risk of developing a disease caused by the expression of a target gene. In this embodiment, iRNA agents can act as novel therapeutic agents for controlling one or more of cellular proliferative and/or differentiative disorders, disorders associated with bone metabolism, immune disorders, hematopoietic disorders, cardiovascular disorders, liver disorders, viral diseases, or metabolic disorders. The method includes administering a pharmaceutical composition of the invention to the patient (e.g., a human), such that expression of the target gene is silenced. Because of their high efficiency and specificity, the iRNA agent of the present invention specifically target mRNA of target genes of diseased cells and tissues, as described below, and at surprisingly low dosages. The pharmaceutical compositions are formulated as described in the preceding section, which is hereby incorporated by reference herein.

Examples of genes which can be targeted for treatment include, without limitation, an oncogene (Hanahan, D. and R. A. Weinberg, Cell (2000) 100:57; and Yokota, J., Carcinogenesis (2000) 21(3):497-503); a cytokine gene (Rubinstein, M., et al., Cytokine Growth Factor Rev. (1998) 9(2):175-81); a idiotype (Id) protein gene (Benezra, R., et al., Oncogene (2001) 20(58):8334-41; Norton, J. D., J. Cell Sci. (2000) 113(22):3897-905); a prion gene (Prusiner, S. B., et al., Cell (1998) 93(3):337-48; Safar, J., and S. B. Prusiner, Prog. Brain Res. (1998) 117:421-34); a gene that expresses molecules that induce angiogenesis (Gould, V. E. and B. M. Wagner, Hum. Pathol. (2002) 33(11):1061-3); adhesion molecules (Chothia, C. and E. Y. Jones, Annu. Rev. Biochem. (1997) 66:823-62; Parise, L. V., et al., Semin. Cancer Biol. (2000) 10(6):407-14); cell surface receptors (Deller, M. C., and Y. E. Jones, Curr. Opin. Struct. Biol. (2000) 10(2):213-9); genes of proteins that are involved in metastasizing and/or invasive processes (Boyd, D., Cancer Metastasis Rev. (1996) 15(1):77-89; Yokota, J., Carcinogenesis (2000) 21(3):497-503); genes of proteases as well as of molecules that regulate apoptosis and the cell cycle (Matrisian, L. M., Curr. Biol. (1999) 9(20):R776-8; Krepela, E., Neoplasma (2001) 48(5):332-49; Basbaum and Werb, Curr. Opin. Cell Biol. (1996) 8:731-738; Birkedal-Hansen, et al., Crit. Rev. Oral Biol. Med. (1993) 4:197-250; Mignatti and Rifkin, Physiol. Rev. (1993) 73:161-195; Stetler-Stevenson, et al., Annu. Rev. Cell Biol. (1993) 9:541-573; Brinkerhoff, E., and L. M. Matrisan, Nature Reviews (2002) 3:207-214; Strasser, A., et al., Annu. Rev. Biochem. (2000) 69:217-45; Chao, D. T. and S. J. Korsmeyer, Annu. Rev. Immunol. (1998) 16:395-419; Mullauer, L., et al., Mutat. Res. (2001) 488(3):211-31; Fotedar, R., et al., Prog. Cell Cycle Res. (1996) 2:147-63; Reed, J. C., Am. J. Pathol. (2000) 157(5):1415-30; D'Ari, R., Bioassays (2001) 23(7):563-5); genes that express the EGF receptor; Mendelsohn, J. and J. Baselga, Oncogene (2000) 19(56):6550-65; Normanno, N., et al., Front. Biosci. (2001) 6:D685-707); and the multi-drug resistance 1 gene, MDR1 gene (Childs, S., and V. Ling, Imp. Adv. Oncol. (1994) 21-36).

In the prevention of disease, the target gene may be one which is required for initiation or maintenance of the disease, or which has been identified as being associated with a higher risk of contracting the disease. In the treatment of disease, the iRNA agent can be brought into contact with the cells or tissue exhibiting the disease. For example, iRNA agent substantially identical to all or part of a mutated gene associated with cancer, or one expressed at high levels in tumor cells, may be brought into contact with or introduced into a cancerous cell or tumor gene.

Examples of cellular proliferative and/or differentiative disorders include cancer, e.g., a carcinoma, sarcoma, metastatic disorder or hematopoietic neoplastic disorder, such as a leukemia. A metastatic tumor can arise from a multitude of primary tumor types, including but not limited to those of prostate, colon, lung, breast and liver origin. As used herein, the terms “cancer,” “hyperproliferative,” and “neoplastic” refer to cells having the capacity for autonomous growth, i.e., an abnormal state or condition characterized by rapidly proliferating cell growth. These terms are meant to include all types of cancerous growths or oncogenic processes, metastatic tissues or malignantly transformed cells, tissues, or organs, irrespective of histopathologic type or stage of invasiveness. Proliferative disorders also include hematopoietic neoplastic disorders, including diseases involving hyperplastic/neoplastic cells of hematopoietic origin, e.g., arising from myeloid, lymphoid or erythroid lineages, or precursor cells thereof.

The pharmaceutical compositions of the present invention can also be used to treat a variety of immune disorders, in particular those associated with overexpression or aberrant expression of a gene or expression of a mutant gene. Examples of hematopoietic disorders or diseases include, without limitation, autoimmune diseases (including, for example, diabetes mellitus, arthritis (including rheumatoid arthritis, juvenile rheumatoid arthritis, osteoarthritis, psoriatic arthritis), multiple sclerosis, encephalomyelitis, myasthenia gravis, systemic lupus erythematosis, autoimmune thyroiditis, dermatitis (including atopic dermatitis and eczematous dermatitis), psoriasis, Sjogren's Syndrome, Crohn's disease, aphthous ulcer, iritis, conjunctivitis, keratoconjunctivitis, ulcerative colitis, asthma, allergic asthma, cutaneous lupus erythematosus, scleroderma, vaginitis, proctitis, drug eruptions, leprosy reversal reactions, erythema nodosum leprosum, autoimmune uveitis, allergic encephalomyelitis, acute necrotizing hemorrhagic encephalopathy, idiopathic bilateral progressive sensorineural hearing, loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Graves' disease, sarcoidosis, primary biliary cirrhosis, uveitis posterior, and interstitial lung fibrosis), graft-versus-host disease, cases of transplantation, and allergy.

In another embodiment, the invention relates to methods for treating viral diseases, including but not limited to hepatitis C, hepatitis B, herpes simplex virus (HSV), HIV-AIDS, poliovirus, and smallpox virus. iRNA agent of the invention are prepared as described herein to target expressed sequences of a virus, thus ameliorating viral activity and replication. The iRNA agents can be used in the treatment and/or diagnosis of viral infected tissue, both animal and plant. Also, such iRNA agent can be used in the treatment of virus-associated carcinoma, such as hepatocellular cancer.

For example, the iRNA agent of the present invention are useful for treating a subject having an infection or a disease associated with the replication or activity of a (+) strand RNA virus having a 3′-UTR, such as HCV. In this embodiment, the iRNA agent can act as novel therapeutic agents for inhibiting replication of the virus. The method includes administering a pharmaceutical composition of the invention to the patient (e.g., a human), such that viral replication is inhibited. Examples of (+) strand RNA viruses which can be targeted for inhibition include, without limitation, picornaviruses, caliciviruses, nodaviruses, coronaviruses, arteriviruses, flaviviruses, and togaviruses. Examples of picornaviruses include enterovirus (poliovirus 1), rhinovirus (human rhinovirus 1A), hepatovirus (hepatitis A virus), cardiovirus (encephalomyocarditis virus), aphthovirus (foot-and-mouth disease virus O), and parechovirus (human echovirus 22). Examples of caliciviruses include vesiculovirus (swine vesicular exanthema virus), lagovirus (rabbit hemorrhagic disease virus), “Norwalk-like viruses” (Norwalk virus), “Sapporo-like viruses” (Sapporo virus), and “hepatitis E-like viruses” (hepatitis E virus). Betanodavirus (striped jack nervous necrosis virus) is the representative nodavirus. Coronaviruses include coronavirus (avian infections bronchitis virus) and torovirus (Berne virus). Arterivirus (equine arteritis virus) is the representative arteriviridus. Togavirises include alphavirus (Sindbis virus) and rubivirus (Rubella virus). Finally, the flaviviruses include flavivirus (Yellow fever virus), pestivirus (bovine diarrhea virus), and hepacivirus (hepatitis C virus). In a preferred embodiment, the virus is hepacivirus, the hepatitis C virus. Although the foregoing list exemplifies vertebrate viruses, the present invention encompasses the compositions and methods for treating infections and diseases caused by any (+) strand RNA virus having a 3′-UTR, regardless of the host. For example, the invention encompasses the treatment of plant diseases caused by sequiviruses, comoviruses, potyviruses, sobemovirus, luteoviruses, tombusviruses, tobavirus, tobravirus, bromoviruses, and closteroviruses.

The pharmaceutical compositions encompassed by the invention may be administered by any means known in the art including, but not limited to, oral or parenteral routes, including intravenous, intramuscular, intraperitoneal, subcutaneous, transdermal, airway (aerosol), ocular, rectal, vaginal, and topical (including buccal and sublingual) administration. In preferred embodiments, the pharmaceutical compositions are administered by intravenous or intraparenteral infusion or injection. The pharmaceutical compositions can also be administered intraparenchymally, intrathecally, and/or by stereotactic injection.

### Genes and Diseases

In one aspect, the invention features, a method of treating a subject at risk for or afflicted with unwanted cell proliferation, e.g., malignant or nonmalignant cell proliferation. The method includes:

providing an iRNA agent, e.g., iRNA agent described herein, e.g., an iRNA having a structure described herein, where the iRNA is homologous to and can silence, e.g., by cleavage, a gene which promotes unwanted cell proliferation;

administering an iRNA agent, e.g., iRNA agent described herein to a subject, preferably a human subject,

thereby treating the subject.

In a preferred embodiment the gene is a growth factor or growth factor receptor gene, a kinase, e.g., a protein tyrosine, serine or threonine kinase gene, an adaptor protein gene, a gene encoding a G protein superfamily molecule, or a gene encoding a transcription factor.

In a preferred embodiment the iRNA agent silences the PDGF beta gene, and thus can be used to treat a subject having or at risk for a disorder characterized by unwanted PDGF beta expression, e.g., testicular and lung cancers.

In another preferred embodiment the iRNA agent silences the Erb-B gene, and thus can be used to treat a subject having or at risk for a disorder characterized by unwanted Erb-B expression, e.g., breast cancer.

In a preferred embodiment the iRNA agent silences the Src gene, and thus can be used to treat a subject having or at risk for a disorder characterized by unwanted Src expression, e.g., colon cancers.

In a preferred embodiment the iRNA agent silences the CRK gene, and thus can be used to treat a subject having or at risk for a disorder characterized by unwanted CRK expression, e.g., colon and lung cancers.

In a preferred embodiment the iRNA agent silences the GRB2 gene, and thus can be used to treat a subject having or at risk for a disorder characterized by unwanted GRB2 expression, e.g., squamous cell carcinoma.

In another preferred embodiment the iRNA agent silences the RAS gene, and thus can be used to treat a subject having or at risk for a disorder characterized by unwanted RAS expression, e.g., pancreatic, colon and lung cancers, and chronic leukemia.

In another preferred embodiment the iRNA agent silences the MEKK gene, and thus can be used to treat a subject having or at risk for a disorder characterized by unwanted MEKK expression, e.g., squamous cell carcinoma, melanoma or leukemia.

In another preferred embodiment the iRNA agent silences the JNK gene, and thus can be used to treat a subject having or at risk for a disorder characterized by unwanted JNK expression, e.g., pancreatic or breast cancers.

In a preferred embodiment the iRNA agent silences the RAF gene, and thus can be used to treat a subject having or at risk for a disorder characterized by unwanted RAF expression, e.g., lung cancer or leukemia.

In a preferred embodiment the iRNA agent silences the Erk1/2 gene, and thus can be used to treat a subject having or at risk for a disorder characterized by unwanted Erk1/2 expression, e.g., lung cancer.

In another preferred embodiment the iRNA agent silences the PCNA(p21) gene, and thus can be used to treat a subject having or at risk for a disorder characterized by unwanted PCNA expression, e.g., lung cancer.

In a preferred embodiment the iRNA agent silences the MYB gene, and thus can be used to treat a subject having or at risk for a disorder characterized by unwanted MYB expression, e.g., colon cancer or chronic myelogenous leukemia.

In a preferred embodiment the iRNA agent silences the c-MYC gene, and thus can be used to treat a subject having or at risk for a disorder characterized by unwanted c-MYC expression, e.g., Burkitt's lymphoma or neuroblastoma.

In another preferred embodiment the iRNA agent silences the JUN gene, and thus can be used to treat a subject having or at risk for a disorder characterized by unwanted JUN expression, e.g., ovarian, prostate or breast cancers.

In another preferred embodiment the iRNA agent silences the FOS gene, and thus can be used to treat a subject having or at risk for a disorder characterized by unwanted FOS expression, e.g., skin or prostate cancers.

In a preferred embodiment the iRNA agent silences the BCL-2 gene, and thus can be used to treat a subject having or at risk for a disorder characterized by unwanted BCL-2 expression, e.g., lung or prostate cancers or Non-Hodgkin lymphoma.

In a preferred embodiment the iRNA agent silences the Cyclin D gene, and thus can be used to treat a subject having or at risk for a disorder characterized by unwanted Cyclin D expression, e.g., esophageal and colon cancers.

In a preferred embodiment the iRNA agent silences the VEGF gene, and thus can be used to treat a subject having or at risk for a disorder characterized by unwanted VEGF expression, e.g., esophageal and colon cancers.

In a preferred embodiment the iRNA agent silences the EGFR gene, and thus can be used to treat a subject having or at risk for a disorder characterized by unwanted EGFR expression, e.g., breast cancer.

In another preferred embodiment the iRNA agent silences the Cyclin A gene, and thus can be used to treat a subject having or at risk for a disorder characterized by unwanted Cyclin A expression, e.g., lung and cervical cancers.

In another preferred embodiment the iRNA agent silences the Cyclin E gene, and thus can be used to treat a subject having or at risk for a disorder characterized by unwanted Cyclin E expression, e.g., lung and breast cancers.

In another preferred embodiment the iRNA agent silences the WNT-1 gene, and thus can be used to treat a subject having or at risk for a disorder characterized by unwanted WNT-1 expression, e.g., basal cell carcinoma.

In another preferred embodiment the iRNA agent silences the beta-catenin gene, and thus can be used to treat a subject having or at risk for a disorder characterized by unwanted beta-catenin expression, e.g., adenocarcinoma or hepatocellular carcinoma.

In another preferred embodiment the iRNA agent silences the c-MET gene, and thus can be used to treat a subject having or at risk for a disorder characterized by unwanted c-MET expression, e.g., hepatocellular carcinoma.

In another preferred embodiment the iRNA agent silences the PKC gene, and thus can be used to treat a subject having or at risk for a disorder characterized by unwanted PKC expression, e.g., breast cancer.

In a preferred embodiment the iRNA agent silences the NFKB gene, and thus can be used to treat a subject having or at risk for a disorder characterized by unwanted NFKB expression, e.g., breast cancer.

In a preferred embodiment the iRNA agent silences the STAT3 gene, and thus can be used to treat a subject having or at risk for a disorder characterized by unwanted STAT3 expression, e.g., prostate cancer.

In another preferred embodiment the iRNA agent silences the survivin gene, and thus can be used to treat a subject having or at risk for a disorder characterized by unwanted survivin expression, e.g., cervical or pancreatic cancers.

In another preferred embodiment the iRNA agent silences the Her2/Neu gene, and thus can be used to treat a subject having or at risk for a disorder characterized by unwanted Her2/Neu expression, e.g., breast cancer.

In another preferred embodiment the iRNA agent silences the topoisomerase I gene, and thus can be used to treat a subject having or at risk for a disorder characterized by unwanted topoisomerase I expression, e.g., ovarian and colon cancers.

In a preferred embodiment the iRNA agent silences the topoisomerase II alpha gene, and thus can be used to treat a subject having or at risk for a disorder characterized by unwanted topoisomerase II expression, e.g., breast and colon cancers.

In a preferred embodiment the iRNA agent silences mutations in the p73 gene, and thus can be used to treat a subject having or at risk for a disorder characterized by unwanted p73 expression, e.g., colorectal adenocarcinoma.

In a preferred embodiment the iRNA agent silences mutations in the p21(WAF1/CIP1) gene, and thus can be used to treat a subject having or at risk for a disorder characterized by unwanted p21(WAF1/CIP1) expression, e.g., liver cancer.

In a preferred embodiment the iRNA agent silences mutations in the p27(KIP1) gene, and thus can be used to treat a subject having or at risk for a disorder characterized by unwanted p27(KIP1) expression, e.g., liver cancer.

In a preferred embodiment the iRNA agent silences mutations in the PPM1D gene, and thus can be used to treat a subject having or at risk for a disorder characterized by unwanted PPM1D expression, e.g., breast cancer.

In a preferred embodiment the iRNA agent silences mutations in the RAS gene, and thus can be used to treat a subject having or at risk for a disorder characterized by unwanted RAS expression, e.g., breast cancer.

In another preferred embodiment the iRNA agent silences mutations in the caveolin I gene, and thus can be used to treat a subject having or at risk for a disorder characterized by unwanted caveolin I expression, e.g., esophageal squamous cell carcinoma.

In another preferred embodiment the iRNA agent silences mutations in the MIB I gene, and thus can be used to treat a subject having or at risk for a disorder characterized by unwanted MIB I expression, e.g., male breast carcinoma (MBC).

In another preferred embodiment the iRNA agent silences mutations in the MTAI gene, and thus can be used to treat a subject having or at risk for a disorder characterized by unwanted MTAI expression, e.g., ovarian carcinoma.

In another preferred embodiment the iRNA agent silences mutations in the M68 gene, and thus can be used to treat a subject having or at risk for a disorder characterized by unwanted M68 expression, e.g., human adenocarcinomas of the esophagus, stomach, colon, and rectum.

In preferred embodiments the iRNA agent silences mutations in tumor suppressor genes, and thus can be used as a method to promote apoptotic activity in combination with chemotherapeutics.

In a preferred embodiment the iRNA agent silences mutations in the p53 tumor suppressor gene, and thus can be used to treat a subject having or at risk for a disorder characterized by unwanted p53 expression, e.g., gall bladder, pancreatic and lung cancers.

In a preferred embodiment the iRNA agent silences mutations in the p53 family member DN-p63, and thus can be used to treat a subject having or at risk for a disorder characterized by unwanted DN-p63 expression, e.g., squamous cell carcinoma

In a preferred embodiment the iRNA agent silences mutations in the pRb tumor suppressor gene, and thus can be used to treat a subject having or at risk for a disorder characterized by unwanted pRb expression, e.g., oral squamous cell carcinoma

In a preferred embodiment the iRNA agent silences mutations in the APC1 tumor suppressor gene, and thus can be used to treat a subject having or at risk for a disorder characterized by unwanted APC1 expression, e.g., colon cancer.

In a preferred embodiment the iRNA agent silences mutations in the BRCA1 tumor suppressor gene, and thus can be used to treat a subject having or at risk for a disorder characterized by unwanted BRCA1 expression, e.g., breast cancer.

In a preferred embodiment the iRNA agent silences mutations in the PTEN tumor suppressor gene, and thus can be used to treat a subject having or at risk for a disorder characterized by unwanted PTEN expression, e.g., hamartomas, gliomas, and prostate and endometrial cancers.

In a preferred embodiment the iRNA agent silences MLL fusion genes, e.g., MLL-AF9, and thus can be used to treat a subject having or at risk for a disorder characterized by unwanted MLL fusion gene expression, e.g., acute leukemias.

In another preferred embodiment the iRNA agent silences the BCR/ABL fusion gene, and thus can be used to treat a subject having or at risk for a disorder characterized by unwanted BCR/ABL fusion gene expression, e.g., acute and chronic leukemias.

In another preferred embodiment the iRNA agent silences the TEL/AML1 fusion gene, and thus can be used to treat a subject having or at risk for a disorder characterized by unwanted TEL/AML1 fusion gene expression, e.g., childhood acute leukemia.

In another preferred embodiment the iRNA agent silences the EWS/FLI1 fusion gene, and thus can be used to treat a subject having or at risk for a disorder characterized by unwanted EWS/FLI1 fusion gene expression, e.g., Ewing Sarcoma.

In another preferred embodiment the iRNA agent silences the TLS/FUS1 fusion gene, and thus can be used to treat a subject having or at risk for a disorder characterized by unwanted TLS/FUS1 fusion gene expression, e.g., Myxoid liposarcoma.

In another preferred embodiment the iRNA agent silences the PAX3/FKHR fusion gene, and thus can be used to treat a subject having or at risk for a disorder characterized by unwanted PAX3/FKHR fusion gene expression, e.g., Myxoid liposarcoma.

In another preferred embodiment the iRNA agent silences the AML1/ETO fusion gene, and thus can be used to treat a subject having or at risk for a disorder characterized by unwanted AML1/ETO fusion gene expression, e.g., acute leukemia.

In another aspect, the invention features, a method of treating a subject, e.g., a human, at risk for or afflicted with a disease or disorder that may benefit by angiogenesis inhibition e.g., cancer. The method includes:

providing an iRNA agent, e.g., an iRNA agent having a structure described herein, which iRNA agent is homologous to and can silence, e.g., by cleavage, a gene which mediates angiogenesis; administering the iRNA agent to a subject, thereby treating the subject.

In a preferred embodiment the iRNA agent silences the alpha v-integrin gene, and thus can be used to treat a subject having or at risk for a disorder characterized by unwanted alpha V integrin, e.g., brain tumors or tumors of epithelial origin.

In a preferred embodiment the iRNA agent silences the Flt-1 receptor gene, and thus can be used to treat a subject having or at risk for a disorder characterized by unwanted Flt-1 receptors, eg. Cancer and rheumatoid arthritis.

In a preferred embodiment the iRNA agent silences the tubulin gene, and thus can be used to treat a subject having or at risk for a disorder characterized by unwanted tubulin, eg. Cancer and retinal neovascularization.

In a preferred embodiment the iRNA agent silences the tubulin gene, and thus can be used to treat a subject having or at risk for a disorder characterized by unwanted tubulin, eg. Cancer and retinal neovascularization.

In another aspect, the invention features a method of treating a subject infected with a virus or at risk for or afflicted with a disorder or disease associated with a viral infection. The method includes:

providing an iRNA agent, e.g., and iRNA agent having a structure described herein, which iRNA agent is homologous to and can silence, e.g., by cleavage, a viral gene of a cellular gene which mediates viral function, e.g., entry or growth; administering the iRNA agent to a subject, preferably a human subject, thereby treating the subject.

Thus, the invention provides for a method of treating patients infected by the Human Papilloma Virus (HPV) or at risk for or afflicted with a disorder mediated by HPV, e.g, cervical cancer. HPV is linked to 95% of cervical carcinomas and thus an antiviral therapy is an attractive method to treat these cancers and other symptoms of viral infection.

In a preferred embodiment, the expression of a HPV gene is reduced. In another preferred embodiment, the HPV gene is one of the group of E2, E6, or E7.

In a preferred embodiment the expression of a human gene that is required for HPV replication is reduced.

The invention also includes a method of treating patients infected by the Human Immunodeficiency Virus (HIV) or at risk for or afflicted with a disorder mediated by HIV, e.g., Acquired Immune Deficiency Syndrome (AIDS).

In a preferred embodiment, the expression of a HIV gene is reduced. In another preferred embodiment, the HIV gene is CCR5, Gag, or Rev.

In a preferred embodiment the expression of a human gene that is required for HIV replication is reduced. In another preferred embodiment, the gene is CD4 or Tsg101.

The invention also includes a method for treating patients infected by the Hepatitis B Virus (HBV) or at risk for or afflicted with a disorder mediated by HBV, e.g., cirrhosis and heptocellular carcinoma.

In a preferred embodiment, the expression of a HBV gene is reduced. In another preferred embodiment, the targeted HBV gene encodes one of the group of the tail region of the HBV core protein, the pre-cregious (pre-c) region, or the cregious (c) region. In another preferred embodiment, a targeted HBV-RNA sequence is comprised of the poly(A) tail.

In preferred embodiment the expression of a human gene that is required for HBV replication is reduced.

The invention also provides for a method of treating patients infected by the Hepatitis A Virus (HAV), or at risk for or afflicted with a disorder mediated by HAV.

In a preferred embodiment the expression of a human gene that is required for HAV replication is reduced.

The present invention provides for a method of treating patients infected by the Hepatitis C Virus (HCV), or at risk for or afflicted with a disorder mediated by HCV, e.g., cirrhosis

In a preferred embodiment, the expression of a HCV gene is reduced.

In another preferred embodiment the expression of a human gene that is required for HCV replication is reduced.

The present invention also provides for a method of treating patients infected by the any of the group of Hepatitis Viral strains comprising hepatitis D, E, F, G, or H, or patients at risk for or afflicted with a disorder mediated by any of these strains of hepatitis.

In a preferred embodiment, the expression of a Hepatitis, D, E, F, G, or H gene is reduced.

In another preferred embodiment the expression of a human gene that is required for hepatitis D, E, F, G or H replication is reduced.

Methods of the invention also provide for treating patients infected by the Respiratory Syncytial Virus (RSV) or at risk for or afflicted with a disorder mediated by RSV, e.g, lower respiratory tract infection in infants and childhood asthma, pneumonia and other complications, e.g., in the elderly.

In a preferred embodiment, the expression of a RSV gene is reduced. In another preferred embodiment, the targeted HBV gene encodes one of the group of genes N, L, or P.

In a preferred embodiment the expression of a human gene that is required for RSV replication is reduced.

Methods of the invention provide for treating patients infected by the Herpes Simplex Virus (HSV) or at risk for or afflicted with a disorder mediated by HSV, e.g, genital herpes and cold sores as well as life-threatening or sight-impairing disease mainly in immunocompromised patients.

In a preferred embodiment, the expression of a HSV gene is reduced. In another preferred embodiment, the targeted HSV gene encodes DNA polymerase or the helicase-primase.

In a preferred embodiment the expression of a human gene that is required for HSV replication is reduced.

The invention also provides a method for treating patients infected by the herpes Cytomegalovirus (CMV) or at risk for or afflicted with a disorder mediated by CMV, e.g., congenital virus infections and morbidity in immunocompromised patients.

In a preferred embodiment, the expression of a CMV gene is reduced.

In a preferred embodiment the expression of a human gene that is required for CMV replication is reduced.

Methods of the invention also provide for a method of treating patients infected by the herpes Epstein Barr Virus (EBV) or at risk for or afflicted with a disorder mediated by EBV, e.g., NK/T-cell lymphoma, non-Hodgkin lymphoma, and Hodgkin disease.

In a preferred embodiment, the expression of a EBV gene is reduced.

In a preferred embodiment the expression of a human gene that is required for EBV replication is reduced.

Methods of the invention also provide for treating patients infected by Kaposi's Sarcoma-associated Herpes Virus (KSHV), also called human herpesvirus 8, or patients at risk for or afflicted with a disorder mediated by KSHV, e.g., Kaposi's sarcoma, multicentric Castleman's disease and AIDS-associated primary effusion lymphoma.

In a preferred embodiment, the expression of a KSHV gene is reduced.

In a preferred embodiment the expression of a human gene that is required for KSHV replication is reduced.

The invention also includes a method for treating patients infected by the JC Virus (JCV) or a disease or disorder associated with this virus, e.g., progressive multifocal leukoencephalopathy (PML).

In a preferred embodiment, the expression of a JCV gene is reduced.

In preferred embodiment the expression of a human gene that is required for JCV replication is reduced.

Methods of the invention also provide for treating patients infected by the myxovirus or at risk for or afflicted with a disorder mediated by myxovirus, e.g., influenza.

In a preferred embodiment, the expression of a myxovirus gene is reduced.

In a preferred embodiment the expression of a human gene that is required for myxovirus replication is reduced.

Methods of the invention also provide for treating patients infected by the rhinovirus or at risk for of afflicted with a disorder mediated by rhinovirus, e.g., the common cold.

In a preferred embodiment, the expression of a rhinovirus gene is reduced.

In preferred embodiment the expression of a human gene that is required for rhinovirus replication is reduced.

Methods of the invention also provide for treating patients infected by the coronavirus or at risk for of afflicted with a disorder mediated by coronavirus, e.g., the common cold.

In a preferred embodiment, the expression of a coronavirus gene is reduced.

In preferred embodiment the expression of a human gene that is required for coronavirus replication is reduced.

Methods of the invention also provide for treating patients infected by the flavivirus West Nile or at risk for or afflicted with a disorder mediated by West Nile Virus.

In a preferred embodiment, the expression of a West Nile Virus gene is reduced. In another preferred embodiment, the West Nile Virus gene is one of the group comprising E, NS3, or NS5.

In a preferred embodiment the expression of a human gene that is required for West Nile Virus replication is reduced.

Methods of the invention also provide for treating patients infected by the St. Louis Encephalitis flavivirus, or at risk for or afflicted with a disease or disorder associated with this virus, e.g., viral haemorrhagic fever or neurological disease.

In a preferred embodiment, the expression of a St. Louis Encephalitis gene is reduced.

In a preferred embodiment the expression of a human gene that is required for St. Louis Encephalitis virus replication is reduced.

Methods of the invention also provide for treating patients infected by the Tick-borne encephalitis flavivirus, or at risk for or afflicted with a disorder mediated by Tick-borne encephalitis virus, e.g., viral haemorrhagic fever and neurological disease.

In a preferred embodiment, the expression of a Tick-borne encephalitis virus gene is reduced.

In a preferred embodiment the expression of a human gene that is required for Tick-borne encephalitis virus replication is reduced.

Methods of the invention also provide for methods of treating patients infected by the Murray Valley encephalitis flavivirus, which commonly results in viral haemorrhagic fever and neurological disease.

In a preferred embodiment, the expression of a Murray Valley encephalitis virus gene is reduced.

In a preferred embodiment the expression of a human gene that is required for Murray Valley encephalitis virus replication is reduced.

The invention also includes methods for treating patients infected by the dengue flavivirus, or a disease or disorder associated with this virus, e.g., dengue haemorrhagic fever.

In a preferred embodiment, the expression of a dengue virus gene is reduced.

In a preferred embodiment the expression of a human gene that is required for dengue virus replication is reduced.

Methods of the invention also provide for treating patients infected by the Simian Virus 40 (SV40) or at risk for or afflicted with a disorder mediated by SV40, e.g., tumorigenesis.

In a preferred embodiment, the expression of a SV40 gene is reduced.

In a preferred embodiment the expression of a human gene that is required for SV40 replication is reduced.

The invention also includes methods for treating patients infected by the Human T Cell Lymphotropic Virus (HTLV), or a disease or disorder associated with this virus, e.g., leukemia and myelopathy.

In a preferred embodiment, the expression of a HTLV gene is reduced. In another preferred embodiment the HTLV1 gene is the Tax transcriptional activator.

In a preferred embodiment the expression of a human gene that is required for HTLV replication is reduced.

Methods of the invention also provide for treating patients infected by the Moloney-Murine Leukemia Virus (Mo-MuLV) or at risk for or afflicted with a disorder mediated by Mo-MuLV, e.g., T-cell leukemia.

In a preferred embodiment, the expression of a Mo-MuLV gene is reduced.

In a preferred embodiment the expression of a human gene that is required for Mo-MuLV replication is reduced.

Methods of the invention also provide for treating patients infected by the encephalomyocarditis virus (EMCV) or at risk for or afflicted with a disorder mediated by EMCV, e.g. myocarditis. EMCV leads to myocarditis in mice and pigs and is capable of infecting human myocardial cells. This virus is therefore a concern for patients undergoing xenotransplantation.

In a preferred embodiment, the expression of a EMCV gene is reduced.

In a preferred embodiment the expression of a human gene that is required for EMCV replication is reduced.

The invention also includes a method for treating patients infected by the measles virus (MV) or at risk for or afflicted with a disorder mediated by MV, e.g. measles.

In a preferred embodiment, the expression of a MV gene is reduced.

In a preferred embodiment the expression of a human gene that is required for MV replication is reduced.

The invention also includes a method for treating patients infected by the Vericella zoster virus (VZV) or at risk for or afflicted with a disorder mediated by VZV, e.g. chicken pox or shingles (also called zoster).

In a preferred embodiment, the expression of a VZV gene is reduced.

In a preferred embodiment the expression of a human gene that is required for VZV replication is reduced.

The invention also includes a method for treating patients infected by an adenovirus or at risk for or afflicted with a disorder mediated by an adenovirus, e.g. respiratory tract infection.

In a preferred embodiment, the expression of an adenovirus gene is reduced.

In a preferred embodiment the expression of a human gene that is required for adenovirus replication is reduced.

The invention includes a method for treating patients infected by a yellow fever virus (YFV) or at risk for or afflicted with a disorder mediated by a YFV, e.g. respiratory tract infection.

In a preferred embodiment, the expression of a YFV gene is reduced. In another preferred embodiment, the preferred gene is one of a group that includes the E, NS2A, or NS3 genes.

In a preferred embodiment the expression of a human gene that is required for YFV replication is reduced.

Methods of the invention also provide for treating patients infected by the poliovirus or at risk for or afflicted with a disorder mediated by poliovirus, e.g., polio.

In a preferred embodiment, the expression of a poliovirus gene is reduced.

In a preferred embodiment the expression of a human gene that is required for poliovirus replication is reduced.

Methods of the invention also provide for treating patients infected by a poxvirus or at risk for or afflicted with a disorder mediated by a poxvirus, e.g., smallpox

In a preferred embodiment, the expression of a poxvirus gene is reduced.

In a preferred embodiment the expression of a human gene that is required for poxvirus replication is reduced.

In another, aspect the invention features methods of treating a subject infected with a pathogen, e.g., a bacterial, amoebic, parasitic, or fungal pathogen. The method includes:

providing a iRNA agent, e.g., a siRNA having a structure described herein, where siRNA is homologous to and can silence, e.g., by cleavage of a pathogen gene;

administering the iRNA agent to a subject, preferably a human subject,

thereby treating the subject.

The target gene can be one involved in growth, cell wall synthesis, protein synthesis, transcription, energy metabolism, e.g., the Krebs cycle, or toxin production.

Thus, the present invention provides for a method of treating patients infected by a plasmodium that causes malaria.

In a preferred embodiment, the expression of a plasmodium gene is reduced. In another preferred embodiment, the gene is apical membrane antigen 1 (AMA1).

In a preferred embodiment the expression of a human gene that is required for plasmodium replication is reduced.

The invention also includes methods for treating patients infected by the Mycobacterium ulcerans, or a disease or disorder associated with this pathogen, e.g. Buruli ulcers.

In a preferred embodiment, the expression of a Mycobacterium ulcerans gene is reduced.

In a preferred embodiment the expression of a human gene that is required for Mycobacterium ulcerans replication is reduced.

The invention also includes methods for treating patients infected by the Mycobacterium tuberculosis, or a disease or disorder associated with this pathogen, e.g. tuberculosis.

In a preferred embodiment, the expression of a Mycobacterium tuberculosis gene is reduced.

In a preferred embodiment the expression of a human gene that is required for Mycobacterium tuberculosis replication is reduced.

The invention also includes methods for treating patients infected by the Mycobacterium leprae, or a disease or disorder associated with this pathogen, e.g. leprosy.

In a preferred embodiment, the expression of a Mycobacterium leprae gene is reduced.

In a preferred embodiment the expression of a human gene that is required for Mycobacterium leprae replication is reduced.

The invention also includes methods for treating patients infected by the bacteria Staphylococcus aureus, or a disease or disorder associated with this pathogen, e.g. infections of the skin and muscous membranes.

In a preferred embodiment, the expression of a Staphylococcus aureus gene is reduced.

In a preferred embodiment the expression of a human gene that is required for Staphylococcus aureus replication is reduced.

The invention also includes methods for treating patients infected by the bacteria Streptococcus pneumoniae, or a disease or disorder associated with this pathogen, e.g. pneumonia or childhood lower respiratory tract infection.

In a preferred embodiment, the expression of a Streptococcus pneumoniae gene is reduced.

In a preferred embodiment the expression of a human gene that is required for Streptococcus pneumoniae replication is reduced.

The invention also includes methods for treating patients infected by the bacteria Streptococcus pyogenes, or a disease or disorder associated with this pathogen, e.g. Strep throat or Scarlet fever.

In a preferred embodiment, the expression of a Streptococcus pyogenes gene is reduced.

In a preferred embodiment the expression of a human gene that is required for Streptococcus pyogenes replication is reduced.

The invention also includes methods for treating patients infected by the bacteria Chlamydia pneumoniae, or a disease or disorder associated with this pathogen, e.g. pneumonia or childhood lower respiratory tract infection

In a preferred embodiment, the expression of a Chlamydia pneumoniae gene is reduced.

In a preferred embodiment the expression of a human gene that is required for Chlamydia pneumoniae replication is reduced.

The invention also includes methods for treating patients infected by the bacteria Mycoplasma pneumoniae, or a disease or disorder associated with this pathogen, e.g. pneumonia or childhood lower respiratory tract infection

In a preferred embodiment, the expression of a Mycoplasma pneumoniae gene is reduced.

In a preferred embodiment the expression of a human gene that is required for Mycoplasma pneumoniae replication is reduced.

In one aspect, the invention features, a method of treating a subject, e.g., a human, at risk for or afflicted with a disease or disorder characterized by an unwanted immune response, e.g., an inflammatory disease or disorder, or an autoimmune disease or disorder. The method includes:

providing an iRNA agent, e.g., an iRNA agent having a structure described herein, which iRNA agent is homologous to and can silence, e.g., by cleavage, a gene which mediates an unwanted immune response;

administering the iRNA agent to a subject,

thereby treating the subject.

In a preferred embodiment the disease or disorder is an ischemia or reperfusion injury, e.g., ischemia or reperfusion injury associated with acute myocardial infarction, unstable angina, cardiopulmonary bypass, surgical intervention e.g., angioplasty, e.g., percutaneous transluminal coronary angioplasty, the response to a transplanted organ or tissue, e.g., transplanted cardiac or vascular tissue; or thrombolysis.

In a preferred embodiment the disease or disorder is restenosis, e.g., restenosis associated with surgical intervention e.g., angioplasty, e.g., percutaneous transluminal coronary angioplasty.

In a preferred embodiment the disease or disorder is Inflammatory Bowel Disease, e.g., Crohn Disease or Ulcerative Colitis.

In a preferred embodiment the disease or disorder is inflammation associated with an infection or injury.

In a preferred embodiment the disease or disorder is asthma, lupus, multiple sclerosis, diabetes, e.g., type II diabetes, arthritis, e.g., rheumatoid or psoriatic.

In particularly preferred embodiments the iRNA agent silences an integrin or co-ligand thereof, e.g., VLA4, VCAM, ICAM.

In particularly preferred embodiments the iRNA agent silences a selectin or co-ligand thereof, e.g., P-selectin, E-selectin (ELAM), I-selectin, P-selectin glycoprotein-1 (PSGL-1).

In particularly preferred embodiments the iRNA agent silences a component of the complement system, e.g., C3, C5, C3aR, C5aR, C3 convertase, C5 convertase.

In particularly preferred embodiments the iRNA agent silences a chemokine or receptor thereof, e.g., TNFI, TNFJ, IL-1I, IL-1J, IL-2, IL-2R, IL-4, IL-4R, IL-5, IL-6, IL-8, TNFRI, TNFRII, IgE, SCYA11, CCR3.

In other embodiments the iRNA agent silences GCSF, Gro1, Gro2, Gro3, PF4, MIG, Pro-Platelet Basic Protein (PPBP), MIP-11, MIP-1J, RANTES, MCP-1, MCP-2, MCP-3, CMBKR1, CMBKR2, CMBKR3, CMBKR5, AIF-1, I-309.

In one aspect, the invention features, a method of treating a subject, e.g., a human, at risk for or afflicted with acute pain or chronic pain. The method includes:

providing an iRNA agent, which iRNA is homologous to and can silence, e.g., by cleavage, a gene which mediates the processing of pain;

administering the iRNA to a subject,

thereby treating the subject.

In particularly preferred embodiments the iRNA agent silences a component of an ion channel.

In particularly preferred embodiments the iRNA agent silences a neurotransmitter receptor or ligand.

In one aspect, the invention features, a method of treating a subject, e.g., a human, at risk for or afflicted with a neurological disease or disorder. The method includes:

providing an iRNA agent which iRNA is homologous to and can silence, e.g., by cleavage, a gene which mediates a neurological disease or disorder;

administering the iRNA agent to a subject,

thereby treating the subject.

In a preferred embodiment the disease or disorder is Alzheimer's Disease or Parkinson Disease.

In particularly preferred embodiments the iRNA agent silences an amyloid-family gene, e.g., APP; a presenilin gene, e.g., PSEN1 and PSEN2, or I-synuclein.

In a preferred embodiment the disease or disorder is a neurodegenerative trinucleotide repeat disorder, e.g., Huntington disease, dentatorubral pallidoluysian atrophy or a spinocerebellar ataxia, e.g., SCA1, SCA2, SCA3 (Machado-Joseph disease), SCA7 or SCA8.

In particularly preferred embodiments the iRNA agent silences HD, DRPLA, SCA1, SCA2, MJD1, CACNL1A4, SCA7, SCA8.

The loss of heterozygosity (LOH) can result in hemizygosity for sequence, e.g., genes, in the area of LOH. This can result in a significant genetic difference between normal and disease-state cells, e.g., cancer cells, and provides a useful difference between normal and disease-state cells, e.g., cancer cells. This difference can arise because a gene or other sequence is heterozygous in euploid cells but is hemizygous in cells having LOH. The regions of LOH will often include a gene, the loss of which promotes unwanted proliferation, e.g., a tumor suppressor gene, and other sequences including, e.g., other genes, in some cases a gene which is essential for normal function, e.g., growth. Methods of the invention rely, in part, on the specific cleavage or silencing of one allele of an essential gene with an iRNA agent of the invention. The iRNA agent is selected such that it targets the single allele of the essential gene found in the cells having LOH but does not silence the other allele, which is present in cells which do not show LOH. In essence, it discriminates between the two alleles, preferentially silencing the selected allele. In essence polymorphisms, e.g., SNPs of essential genes that are affected by LOH, are used as a target for a disorder characterized by cells having LOH, e.g., cancer cells having LOH.

E.g., one of ordinary skill in the art can identify essential genes which are in proximity to tumor suppressor genes, and which are within a LOH region which includes the tumor suppressor gene. The gene encoding the large subunit of human RNA polymerase II, POLR2A, a gene located in close proximity to the tumor suppressor gene p53, is such a gene. It frequently occurs within a region of LOH in cancer cells. Other genes that occur within LOH regions and are lost in many cancer cell types include the group comprising replication protein A 70-kDa subunit, replication protein A 32-kD, ribonucleotide reductase, thymidilate synthase, TATA associated factor 2H, ribosomal protein S14, eukaryotic initiation factor 5A, alanyl tRNA synthetase, cysteinyl tRNA synthetase, NaK ATPase, alpha-1 subunit, and transferrin receptor.

Accordingly, the invention features, a method of treating a disorder characterized by LOH, e.g., cancer. The method includes:

optionally, determining the genotype of the allele of a gene in the region of LOH and preferably determining the genotype of both alleles of the gene in a normal cell;

providing an iRNA agent which preferentially cleaves or silences the allele found in the LOH cells;

administering the iRNA to the subject,

thereby treating the disorder.

The invention also includes a iRNA agent disclosed herein, e.g, an iRNA agent which can preferentially silence, e.g., cleave, one allele of a polymorphic gene

In another aspect, the invention provides a method of cleaving or silencing more than one gene with an iRNA agent. In these embodiments the iRNA agent is selected so that it has sufficient homology to a sequence found in more than one gene. For example, the sequence AAGCTGGCCCTGGACATGGAGAT (SEQ ID NO:33) is conserved between mouse lamin B1, lamin B2, keratin complex 2-gene 1 and lamin A/C. Thus an iRNA agent targeted to this sequence would effectively silence the entire collection of genes.

### DEFINITIONS

The term “alkyl” refers to saturated and unsaturated non-aromatic hydrocarbon chains that may be a straight chain or branched chain, containing the indicated number of carbon atoms (these include without limitation propyl, allyl, or propargyl), which may be optionally inserted with N, O, or S. For example, C₁-C₂₀ indicates that the group may have from 1 to 20 (inclusive) carbon atoms in it. The term “alkoxy” refers to an —O-alkyl radical. The term “alkylene” refers to a divalent alkyl (i.e., —R—). The term “alkylenedioxo” refers to a divalent species of the structure —O—R—O—, in which R represents an alkylene. The term “aminoalkyl” refers to an alkyl substituted with an amino. The term “mercapto” refers to an —SH radical. The term “thioalkoxy” refers to an —S-alkyl radical.

The term “aryl” refers to a 6-carbon monocyclic or 10-carbon bicyclic aromatic ring system wherein 0, 1, 2, 3, or 4 atoms of each ring may be substituted by a substituent. Examples of aryl groups include phenyl, naphthyl and the like. The term “arylalkyl” or the term “aralkyl” refers to alkyl substituted with an aryl. The term “arylalkoxy” refers to an alkoxy substituted with aryl.

The term “cycloalkyl” as employed herein includes saturated and partially unsaturated cyclic hydrocarbon groups having 3 to 12 carbons, for example, 3 to 8 carbons, and, for example, 3 to 6 carbons, wherein the cycloalkyl group additionally may be optionally substituted. Cycloalkyl groups include, without limitation, cyclopropyl, cyclobutyl, cyclopentyl, cyclopentenyl, cyclohexyl, cyclohexenyl, cycloheptyl, and cyclooctyl.

The term “heteroaryl” refers to an aromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively), wherein 0, 1, 2, 3, or 4 atoms of each ring may be substituted by a substituent. Examples of heteroaryl groups include pyridyl, furyl or furanyl, imidazolyl, benzimidazolyl, pyrimidinyl, thiophenyl or thienyl, quinolinyl, indolyl, thiazolyl, and the like. The term “heteroarylalkyl” or the term “heteroaralkyl” refers to an alkyl substituted with a heteroaryl. The term “heteroarylalkoxy” refers to an alkoxy substituted with heteroaryl.

The term “heterocyclyl” refers to a nonaromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively), wherein 0, 1, 2 or 3 atoms of each ring may be substituted by a substituent. Examples of heterocyclyl groups include piperazinyl, pyrrolidinyl, dioxanyl, morpholinyl, tetrahydrofuranyl, and the like.

The term “oxo” refers to an oxygen atom, which forms a carbonyl when attached to carbon, an N-oxide when attached to nitrogen, and a sulfoxide or sulfone when attached to sulfur.

The term “acyl” refers to an alkylcarbonyl, cycloalkylcarbonyl, arylcarbonyl, heterocyclylcarbonyl, or heteroarylcarbonyl substituent, any of which may be further substituted by substituents.

The term “substituents” refers to a group “substituted” on an alkyl, cycloalkyl, aryl, heterocyclyl, or heteroaryl group at any atom of that group. Suitable substituents include, without limitation, halo, hydroxy, oxo, nitro, haloalkyl, alkyl, alkaryl, aryl, aralkyl, alkoxy, aryloxy, amino, acylamino, alkylcarbamoyl, arylcarbamoyl, aminoalkyl, alkoxycarbonyl, carboxy, hydroxyalkyl, alkanesulfonyl, arenesulfonyl, alkanesulfonamido, arenesulfonamido, aralkylsulfonamido, alkylcarbonyl, acyloxy, cyano, and ureido groups.

### EXAMPLES

The invention now being generally described, it will be more readily understood by reference to the following examples, which are included merely for purposes of illustration of certain aspects and embodiments of the invention, and are not intended to limit the invention. Thus, the invention should in way be construed as being limited to the following examples, but rather, should be construed to encompass any and all variations which become evident as a result of the teaching provided herein.

### Example 1

### Preparation of the Precursor Phosphorothioate Oligonucleotide as a Carrier for siRNA Targeting PCSK9

TABLE 1
Sequence information for the phosphorothioate
carrier oligonucleotide A-30829.

| Target | Seq. ID | Sequence (5′ to 3′) | MW_{calc} |
|---|---|---|---|
| N/A | A-30829 | HO—(CH₂)₆—S—S—(CH₂)₆-ps- | 6732.69 |
| | | dCsdTsdTsdAsdCsdGsdCsd | |
| | | TsdGsdAsdGsdTsdAsdCsd | |
| | | TsdTsdCsdGsdAsdT | |
| | | (SEQ ID NO: 1) | |

| | | | |
|---|---|---|---|
| N/A | A-30829 | Py-S—S—(CH₂)₆-ps- | 6709.7 |
| | (Py-S—S) | dCsdTsdTsdAsdCsdGsdCsd | |
| | | TsdGsdAsdGsdTsdAsdCsd | |
| | | TsdTsdCsdGsdAsdT | |
| | | (SEQ ID NO: 1) | |

Py: pyridyl;
s: phosphorothioate linkage

The synthesis of the carrier oligo was carried out using an Applied Biosystems DNA/RNA Synthesizer 394 and standard phosphoramidite chemistry in a scale of ca. 0.1 mmol. To introduce the thiol on the 5′-end, the commercially available Thiol-Modifier C6 S-S from Glen Research was used as described in the manufacturer's protocol. After deprotection with aq. ammonia for 5 hours at 55° C. and evaporation of the ammonia solution, the crude product was purified by reverse phase HPLC on a DeltaPak C18 column (19×250 mm) using 0.05 M TEAAc buffer (eluent A) and acetonitrile (eluent B). The pure fractions were combined and diluted to about 100 mL with water before 1.54 g of DTT (dithiothreitol) was added to reduce the disulfide to the free thiol under shaking at about 40° C. The reaction was monitored by RP-HPLC and upon completion, excess salt and DTT was removed by size exclusion chromatography using Sephadex G25. The fractions containing the oligonucleotide were combined and 195 mg dipyridyldisulfide in 5 mL acetonitrile were added. After shaking the reaction mixture for about 1 hour, the solvent was evaporated using a rotary evaporator. The residual solid was re-dissolved in water and extracted 10 times with diethylether. After evaporation of the residual ether from the aqueous phase, the sample was purified by RP-HPLC on a DeltaPak C18 column (19×250 mm) using 0.05 M TEAAc buffer (eluent A) and acetonitrile (eluent B). The pure fractions were combined, lyophilized and stored at −20° C. prior to further use.

### Example 2

### Preparation of the Precursor siRNA for Conjugation to the PTO Carrier

TABLE 2
Sequence information for the
thiol-modified sense strand A-30681.

| Target | Seq. ID | Sequence (5′ to 3′) | MW_{calc} |
|---|---|---|---|
| PCSK9^{a} | 30681 | GccuGGAGuuuAuucGGAAQ51pdT | 6837.5 |
| | | (SEQ ID NO: 34) | |
| PCSK9 | 30681- | GccuGGAGuuuAuucGGAAp- | 6405.5 |
| | SH | (CH2)6—SH | |
| | | (SEQ ID NO: 34) | |

Upper case: RNA;
lower case 2′-0-methyl;
Q51: (CH2)6—S—S—(CH2)6 linker

The synthesis of the A-30681 was carried out using an Aekta Oliopilot100 DNA/RNA Synthesizer and standard phosphoramidite chemistry in a scale of ca. 0.2 mmol. To introduce the thiol on the 3′-end, the commercially available Thiol-Modifier C6 S-S from Glen Research was used as described in the manufacturer's protocol and coupled onto a LCAA-CPG resin loaded with dT succinate. After deprotection with aq. methylamine for 90 hours at 65° C. followed by pyridine-HF treatment, the solution was diluted with eluent A and the crude product was purified by strong anion exchange HPLC using a 5 cm column loaded with SuperQ-5PW (20) (Tosoh Biosciences) and 20 mM Na-phosphate pH 8.5 in water/acetonitrile (9:1) as eluent A and 20 mM Na-phosphate, 1 M NaBr pH 8.5 in water/acetonitrile (9:1) as eluent B. For disulfide reduction, 30681 was dissolved in 13 mL of 0.05 M NH4OAc buffer (pH 7) at a concentration of 1 mM and 4.16 mL of 100 mM Tris(2-carboxyethyl)phosphine hydrochloride (TCEP) in 0.05 M NH4OAc was added. The reaction mixture was shaken at 45° C. for 90 min and the excess TCEP was removed by size exclusion chromatography using Sephadex G25. The fractions containing the oligonucleotide were collected and evaporated to dryness. After re-dissolving the product in 0.05 M TEAAc buffer (pH 7), the sample was further purified by RP-HPLC on a DeltaPak C18 column (19×250 mm) using 0.05 M TEAAc buffer (eluent A) and acetonitrile (eluent B). The pure fractions were combined, evaporated to dryness and stored at −20° C. prior to further use.

### Example 3

### Preparation of the Phosphorothioate Oligonucleotide-siRNA Conjugate

TABLE 3
Sequence information for the
modified sense strand A-30925.

| | Seq. | | |
|---|---|---|---|
| Target | ID | Sequence (5′ to 3′) | MW_{calc} |
| PCSK9^{a} | A- | GccuGGAGuuuAuucGGAAQ51dCsdTsd | 12998.5 |
| | 30925 | TsdAsdCsdGsdCsdTsdGsdAsdGsdTsd | |
| | | AsdCsdTsdTsdCsdGsdAsdT | |
| | | (SEQ ID NOs: 34, 1) | |

Upper case: RNA;
lower case 2′-0-methyl;
s: phosphorothioate linkage;
Q51: (CH2)6—S—S—(CH2)6 linker

8.6 μmol of 30681-SH was dissolved in degassed water at a concentration of 1.025 mM and 8.6 μmol of Py-S-S-30829 dissolved in degassed water at a concentration of 2 mM was added under shaking. 5M NH4OAc buffer (pH 7) was added to a final concentration of 0.1 M and the mixture was shaken at 45° C. for 24 hours. The conjugate was purified by RP-HPLC on a DeltaPak C18 column (19×250 mm) using 0.05 M TEAAc buffer (eluent A) and acetonitrile (eluent B). The pure fractions were combined desalted by ultraflitration using a Hydrosart 2K cut off membrane (Sartorius), lyophilized and stored at −20° C. prior to further use.

The modified sense strand was annealed to its complementary antisense strand in 1×PBS solution by adding equal molar amounts of the two strands and heating the solution to 95° C. for 5 min (Table 4). After the mixture has been allowed to slowly cool to room temperature, it was stored at 4° C. until further use.

TABLE 4
Sequence information for the
modified duplex AD-3682.

| Target | Duplex | Strand | Sequence (5′ to 3′) | MW_{calc} |
|---|---|---|---|---|
| PCSK9^{a} | AD- | Sense | GccuGGAGuuuAuucGGAAp | 12998.5 |
| | 3682 | | Q51dCsdTsdTsdAsdCsd | 6657.14 |
| | | | GsdCsdTsdGsdAsdGsdTs | |
| | | | dAsdCsdTsdTsdCsdGsdAsdT | |
| | | | (SEQ ID NOs: 34, 1) | |
| | | Anti- | UUCCGAAuAAACUCcAGGCdTsdT | |
| | | sense | (SEQ ID NOs: 35) | |

Uppercase: RNA;
lower case 2′-O-methyl.
s: phosphorothioate;
Q51: (CH2)6—S—S—(CH2)6 linker

### Example 4

### In Vivo Evaluation of AD-3682 in Mice

C57/BL6 mice (5/group, 8-10 weeks old, Charles River Laboratories, MA) were dosed with siRNAs formulated in PBS-solution by bolus low volume tail vein injection using a 27 G needle or via interperotineal IP injection. Dosing was carried out two times per week at 50 mg/kg for three weeks. Mice were kept under an infrared lamp for approximately 3 min prior to dosing to ease injection. 48 hour post last dose mice were sacrificed by CO₂-asphyxiation. 0.2 ml blood was collected by retro-orbital bleeding and the liver was harvested and frozen in liquid nitrogen. Serum and livers were stored at −80° C. Total serum cholesterol in mouse serum was measured using the StanBio Cholesterol LiquiColor kit (StanBio Laboratoriy, Boerne, Tex., USA) according to manufacturer's instructions. Measurements were taken on a Victor2 1420 Multilabel Counter (Perkin Elmer) at 495 nm. Message levels of the target gene PCSK9 were measured via bDNA analysis as below.

### Example 5

### bDNA Analysis of Liver

Frozen livers were grinded using 6850 Freezer/Mill Cryogenic Grinder (SPEX CentriPrep, Inc) and powders stored at −80° C. until analysis.

PCSK9 mRNA levels were detected using the branched-DNA technology based kit from QuantiGene Reagent System (Genospectra version 1 or 2) according to the protocol. 10-20 mg of frozen liver powders was lysed in 600 ul of 0.16 ug/ml Proteinase K (Epicentre, #MPRK092) in Tissue and Cell Lysis Solution (Epicentre, #MTC096H) at 65° C. for 3 hours. Then 10 ul of the lysates were added to 90 ul of Lysis Working Reagent (1 volume of stock Lysis Mixture in two volumes of water) and incubated at 52° C. overnight on Genospectra capture plates with probe sets specific to mouse PCSK9 and mouse GAPDH. Nucleic acid sequences for Capture Extender (CE), Label Extender (LE) and blocking (BL) probes were selected from the nucleic acid sequences of PCSK9 and GAPDH with the help of the QuantiGene ProbeDesigner Software 2.0 (Genospectra, Fremont, Calif., USA, cat. No. QG-002-02). Chemo luminescence was read on a Victor2-Light (Perkin Elmer) as Relative light units. The ratio of PCSK9 mRNA to GAPDH mRNA in liver lysates was averaged over each treatment group and compared to a control group treated with PBS.

As shown in Table 5, as compared to the PBS control, compounds AD-3682 resulted in significant (˜72%) lowering of PCSK9 transcript levels in mouse liver, (as indicated by a smaller PCSK9 to GAPDH transcript ratio, when normalized to a PBS control group), indicating that the conjugated siRNA molecules were active in vivo. The silencing activity observed resulted in lowering of total cholesterol in those animals. The results were similar whether the animals were dosed IV or IP.

TABLE 5
Silencing of PCSK9 message and reduction of serum cholesterol by AD-3682 after i.v. or
i.p. injection; values are normalized to GAPDH or PBS-treated animals, respectively.

| | | | Liver | | | | Serum | | | |
|---|---|---|---|---|---|---|---|---|---|---|
| | Sense | Antisense | IV | | IP | | IV | | IP | |
| | strand | strand | PCSK9/GAPDH | SD | PCSK9/GAPDH | SD | Cholesterol | SD | Cholesterol | SD |
| PBS | | | 1.00 | 0.24 | 1.00 | 0.21 | 1.00 | 0.09 | 1.00 | 0.05 |
| AD-3682 | A-30925 | A-16866 | 0.28 | 0.07 | 0.55 | 0.19 | 0.55 | 0.07 | 0.37 | 0.10 |

C57BL6 N = 5/group
2 × week × 50 mg/kg for 3 weeks, Sac 1 day (IV) and 2 days (IP) post last injection
All data normalized to PBS control

### Example 6

### Synthesis of PTO Carrier Oligo Attached to Dicer Substrate siRNA

TABLE 6
Sequence information for the
modified duplex AD-3814.

| Target | Duplex | Strand | Sequence (5′ to 3′) | MW_{calc} |
|---|---|---|---|---|
| PCSK9^{a} | AD- | Sense | GccuGGAGuuuAuucGG | 8873.57 |
| | 3814 | | AAGA\|GucAGsc | |
| | | | (SEQ ID NO: 36) | |
| | | Anti- | dTsdGsdGsdAsdGsdT | 15829.2 |
| | | sense | sdGsdTsdCsdTsdAsd | |
| | | | CsdCsdAsdCsdTsdAs | |
| | | | dTsdCsdT- | |
| | | | GcuGAcuC\|UUCCGAAu | |
| | | | AAACUCcAGGCcsu | |
| | | | (SEQ ID NO: 37) | |

Upper case: RNA;
lower case 2′-O-methyl;
s: phosphorothioate;
dC: 5-methyl-2′-deoxycytidine;
| putative dicer cleavage site

The synthesis of the chimeric antisense strand containing the 20mer phosphorothioate carrier and the 29mer RNA with the putative dicer cleavage site was carried out by solid phase synthesis on a CPG resin pre-loaded with 2′-O-methyl-uridine (Prime Synthesis) using an Applied Biosystems DNA/RNA Synthesizer 394 and commercially available DNA and RNA amidites (ChemGenes) at a scale of ca. 4×0.025 mmol. After deprotection with aq. ammonia/ethanol (3:1) for 5 hours at 55° C. followed by pyridine-HF treatment, the solution was diluted with eluent A and the crude product was purified by strong anion exchange HPLC using a 5 cm column loaded with SuperQ-5PW (20) (Tosoh Biosciences) and 20 mM Na-phosphate pH 8.5 in water/acetonitrile (9:1) as eluent A and 20 mM Na-phosphate, 1 M NaBr pH 8.5 in water/acetonitrile (9:1) as eluent B. Fractions containing the full length antisense strand were further purified by reverse phase HPLC on a DeltaPak C18 column (19×250 mm) using 0.05 M TEAAc buffer (eluent A) and acetonitrile (eluent B). The pure fractions were combined and desalted by ultraflitration using a Hydrosart 2K cut off membrane (Sartorius), lyophilized and stored at −20° C. prior to further use.

The sense strand was synthesized, purified and characterized using standard procedures for RNA oligonucleotides and annealed with the antisense strand in 1×PBS buffer by heating equimolar amounts of both strands in the buffered solution to 90° C. and then allowing the solution to cool down over a period of several hours. The duplex was stored at −20° C. prior to further use.

### Example 7

### 10792-Based PCSK9 siRNAs with Carrier Oligonucleotides

### Experimental Design:

C57Bl/6 mice, N=5/group

50 mg/kg×2 per week for 3 weeks, all siRNAs @ 0.372 mM
Table 7 shows the siRNAs used

inject 10 ul/g, both IV and IP
iv: 7 groups×5/group->35
ip: 7 groups×5/group->35′+10->45

inject on day 0, 3, 6, 9, 13 and 15
for iv sac on day 16
for ip sac on day 17

day 16 and 17: blood draws, liver, jejunum, ileum, colon, kidneys, spleen . . . .

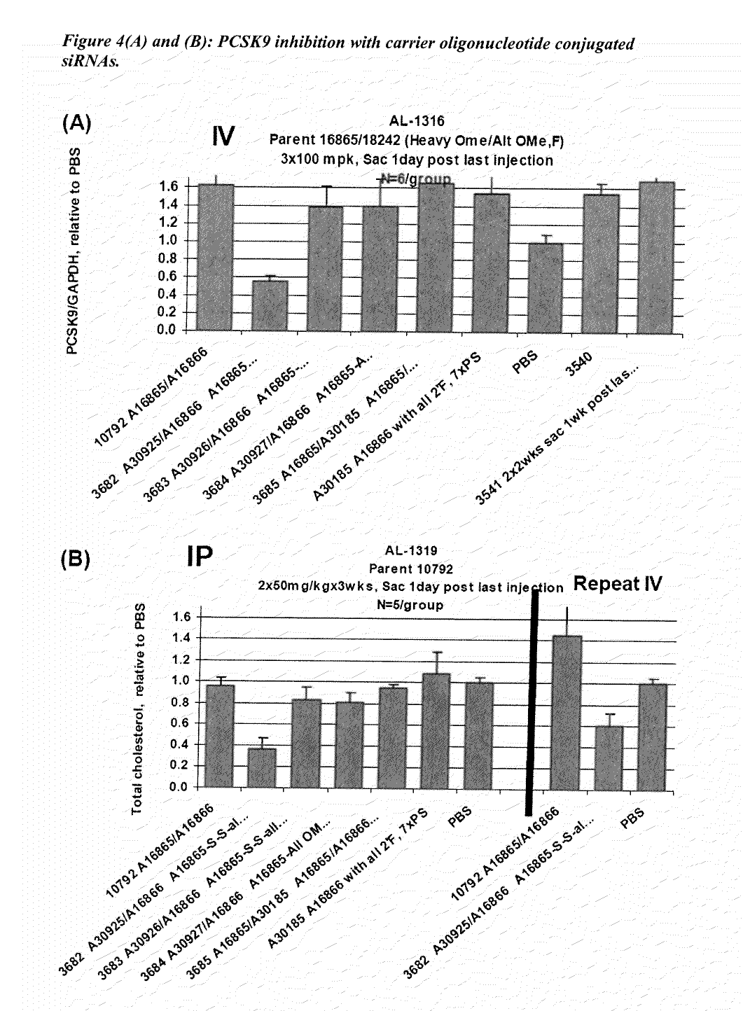

Results are shown in FIGS. 4 (A) and (B).

TABLE 7
Sequence information of 10792-based siRNAs targeting PCSK9.

| Duplex | Strand | S/AS | Sequence (5′->3′)* |
|---|---|---|---|
| AD-10792 | A16865 | S | GccuGGAGuuuAuucGGAAdTsdT (SEQ ID NO: 38) |
| | A16866 | AS | UUCCGAAuAAACUCcAGGCdTsdT (SEQ ID NO: 35) |
| AD-3682 | A30925 | S | GccuGGAGuuuAuucGGAAQ51sdCsdTsdTsdAsdCsdGsd |
| | | | CsdTsdGsdAsdGsdTsdAsdCsdTsdTsdCsdGsdAsdT |
| | | | (SEQ ID NOs: 34, 1) |
| | A16866 | AS | UUCCGAAuAAACUCcAGGCdTsdT (SEQ ID NO: 35) |
| AD-3683 | A30926 | S | GccuGGAGuuuAuucGGAAQ51scsusuacgcugaguacuucs |
| | | | gsasusL10 (SEQ ID NOs: 34, 39) |
| | A16866 | AS | UUCCGAAuAAACUCcAGGCdTsdT (SEQ ID NO: 35) |
| AD-3684 | A30927 | S | GccuGGAGuuuAuucGGAAcsusuacgcugaguacuucsgsa |
| | | | susL10 (SEQ ID NOs: 34, 39) |
| | A16866 | AS | UUCCGAAuAAACUCcAGGCdTsdT (SEQ ID NO: 35) |
| AD-3685 | A16865 | S | GccuGGAGuuuAuucGGAAdTsdT (SEQ ID NO: 38) |
| | A30185 | AS | pUfUfCfCfGfAfAfUfAfAfAfCfsUfsCfsCfsAfsGfs |
| | | | GfsCf (SEQ ID NOs: 40) |

s: phosphorothioate linkage;
lower case: 2′-O-methyl;
upper case: RNA;
Nf: 2′-fluoro nucleoside;
Q51: (CH2)6—S—S—(CH2)6 linker;
L10: cholesterol;
p: 5′-phosphate

### Example 8

### Preparation of an Abasic Precursor Phosphorothioate Oligomer as a Carrier for siRNA Targeting PCSK9

The synthesis of the carrier oligo is carried out using an Applied Biosystems DNA/RNA Synthesizer 394 and the commercially available abasic phosphoramidite (Glen Research) in a scale of ca. 4×0.025 mmol. To introduce the thiol on the 5′-end, the commercially available Thiol-Modifier C6 S-S (Glen Research) is used as described in the manufacturer's protocol. After deprotection with aq. ammonia for 5 hours at 55° C. and evaporation of the ammonia solution, the crude product is purified by reverse phase HPLC on a DeltaPak C18 column (19×250 mm) using 0.05 M TEAAc buffer (eluent A) and acetonitrile (eluent B). The pure fractions are combined and diluted to about 100 mL with water before 1.54 g of DTT (dithiothreitol) and 1.5 mL of 100 mM Tris(2-carboxyethyl)phosphine hydrochloride (TCEP) in 0.05 M NH4OAc are added to reduce the disulfide to the free thiol under shaking at 40° C. The reaction is monitored by RP-HPLC and upon completion, excess salt and DTT are removed by size exclusion chromatography using Sephadex G25. The fractions containing the oligomer are combined and 200 mg dipyridyldisulfide in 5 mL acetonitrile are added. After shaking the reaction mixture for about 1 hour, the solvent is evaporated using a rotary evaporator. The residual solid is re-dissolved in 25 mL water and extracted 10 times with diethylether. After evaporation of the residual ether from the aqueous phase, the sample is purified by RP-HPLC on a DeltaPak C18 column (19×250 mm) using 0.05 M TEAAc buffer (eluent A) and acetonitrile (eluent B). The pure fractions are combined, lyophilized and stored at −20° C. prior to further use.

### Example 9

### Preparation of the Abasic Phosphorothioate Oligomer-siRNA Conjugate

TABLE 8
Sequence information for the modified sense strand.

| Target | Sequence (5′ to 3′) | MW_{calc} |
|---|---|---|
| PCSK9^{a} | GccuGGAGuuuAuucGGAAQ51s(dXs)₁₉dX | 10435.4 |
| | (SEQ ID NOs: 34, 41) | |

Upper case: RNA;
lower case 2′-O-methyl;
s: phosphorothioate linkage,
dX: 1′,2′-Dideoxyribose;
Q51: (CH2)6—S—S—(CH2)6 linker

5 μmol of 30681-SH are dissolved in degassed water at a concentration of 1.0 mM and 5 μmol of the carrier oligo containing the pyridyldisulfide are dissolved in degassed water at a concentration of 2 mM. The two solutions are combined under shaking, 5M NH4OAc buffer (pH 7) is added to a final concentration of 0.1 M and the mixture is shaken at 45° C. for 24 hours. The conjugate is purified by RP-HPLC on a DeltaPak C18 column (19×250 mm) using 0.05 M TEAAc buffer (eluent A) and acetonitrile (eluent B). The pure fractions are combined, desalted by ultraflitration using a Hydrosart 2K cut off membrane (Sartorius), lyophilized and stored at −20° C. prior to further use.

The modified sense strand is annealed to its complementary antisense strand in 1×PBS solution by adding equal molar amounts of the two strands and heating the solution to 95° C. for 5 min (Table 4). The mixture is allowed to slowly cool to room temperature and is stored at 4° C. until further use.

TABLE 9
Sequence information for the
modified duplex AD-3682.

| Target | Duplex | Strand | Sequence (5′ to 3′) | MW_{calc} |
|---|---|---|---|---|
| PCSK9^{a} | AD- | Sense | GccuGGAGuuuAuucGGAA- | 12998.5 |
| | 3682 | | Q51s-(dXs)₁₉dX | 6657.14 |
| | | | (SEQ ID NOs: 34, 41) | |
| | | Anti- | UUCCGAAuAAACUCcAGGCd | |
| | | sense | TsdT (SEQ ID NO: 35) | |

Upper case: RNA;
lower case 2′-O-methyl;
s: phosphorothioate;
dX: 1′,2′-Dideoxyribose;
Q51: (CH2)6—S—S—(CH2)6 linker

### Example 10

### Some Poly(G) Carrier Oligonucleotide siRNAs

The ebola oligonucleotide with polydeoxyguanosine at 3′ end was synthesized on ABI394 using the standard phosphoramidite chemistry. Methylamine (40% Aqueous) was used for cleavage of the RNA from CPG support and base deprotection. Triethylamine trihydrofluoride was used to remove 2′ TBDMS group. It was purified with ion-exchange HPLC and the fractions were analyzed with LC/MS. Pure fractions were then combined and desalted on size-exclusion column. Final siRNA duplex was annealed in PBS buffer.

TABLE 10
Sequence information for poly(G)
modified oligonucleotides

| | | Strand | |
|---|---|---|---|
| Duplex | Strand | ID | Sequence (5′ to 3′) |
| Ad-3938-b1 | Sense | 31004 | cuGGcuGAAuuucAGAGcA(dG)₁₀ |
| | | | (SEQ ID NO: 42) |
| | Anti- | 22826 | UGCUCUGAAAUUcAGCcAGTsT |
| | sense | | (SEQ ID NO: 43) |
| AD-3939-b1 | Sense | 31014 | cuGGcuGAAuuucAGAGcA(dG)₁₀ |
| | | | (SEQ ID NO: 42) |
| | Anti- | 31004 | ugcucugaaauucagccagdTsdT |
| | sense | | (SEQ ID NO: 44) |
| AD-3940-b1 | Sense | 31004 | cuGGcuGAAuuucAGAGcA(dG)₁₀ |
| | | | (SEQ ID NO: 42) |
| | Anti- | 31005 | ugcucugaaauucagccagdTsdT- |
| | sense | | OrG488 (SEQ ID NO: 44) |

Upper case: RNA;
lower case 2′-O-methyl;
s: phosphorothioate;
OrG488: Oregon Green

### Example 11

### Preparation of siRNA-Carrier Oligonucleotide Conjugates by Solid Phase Synthesis

The siRNA sense strands conjugated to all phosphorothioate carrier oligos, which are listed in Table 11, were synthesized by continuous solid phase synthesis as described below.

TABLE 11
Sequence information of siRNA sense strand conjugates synthesized
by continuous solid phase synthesis

| | Strand | | |
|---|---|---|---|
| Target | ID | Strand | Sequence (5′ to 3′) |
| PCSK9 | 33574 | S | GfcCfuGfgAfgUfuUfaUfuCfgGfaAfdTsdTsQ50sds(CTTACGCT |
| | | | GAGTACTTCGAT) (SEQ ID NOs: 45, 46) |
| FVII | 33575 | S | GfgAfuCfaUfcUfcAfaGfuCfuUfaCfdTsdTsQ50sds(CTTACGCT |
| | | | GAGTACTTCGAT) (SEQ ID NOs: 47, 46) |
| PCSK9 | 33591 | S | GfcCfuGfgAfgUfuUfaUfuCfgGfaAfdTsdIsQ50ds(GCCCAAGCT |
| | | | GGCATCCGTCA) (SEQ ID NOs: 45, 48) |
| FVII | 33592 | S | GfgAfuCfaUfcUfcAfaGfuCfuUfaCfdTsdTsQ50ds(GCCCAAGCT |
| | | | GGCATCCGTCA) (SEQ ID NOs: 47, 48) |

s: phosphorothioate linkage;
lower case: 2′-O-methyl;
upper case: RNA;
Nf: 2′-fluoro nucleoside;
C: 5-methylcytidine;
Q50: (CH2)12 linker;
Q51: (CH2)6—S—S—(CH2)6 linker;
L10: cholesterol.

The synthesis of the siRNA sense strands containing alternating 2′-fluoro/2′-O-methyl modifications conjugated to 20mer phosphorothioate carriers was carried out by solid phase synthesis on a CPG resin pre-loaded with 2′-deoxy A or T (Prime Synthesis) using an Applied Biosystems DNA/RNA Synthesizer 394 and commercially available DNA and RNA amidites (ChemGenes) at a scale of ca. 4×0.025 mmol. The DMT-group of the last nucleotide incorporated on the 5′-end was kept on. After deprotection with aq. Ammonia at 55 C for 6 hours, the crude product was evaporated to dryness on a rotavap and the dry product was dissolved in water and filtered into a 50 mL plastic tube prior to purification. The crude product weal purified by preparative HPLC using a DeltaPak C18 column (19×300 mm). 0.1 M NH₄OAc and acetonitrile were used as eluents A and B, respectively and the product was eluted using a gradient of 0-50% B in 35 min. After purification, m the fractions containing the pure product were combined and evaporated and the DMT group was removed by treating the dry sample with 80% aqueous acetic acid for 30 min. Then the sample was evaporated again on the rotavap and redissolved in 0.1 M triethylammonium acetate buffer for 2^{nd} HPLC purification using 50 mM NaOAc buffer in water and in 80% acetonitrile as eluents A and B, respectively. The fractions containing pure detritylated product were combined and desalted by ultrafiltration.

### Example 12

### Immunostimulatory Effect of siRNA Conjugates in hPBMC Assay

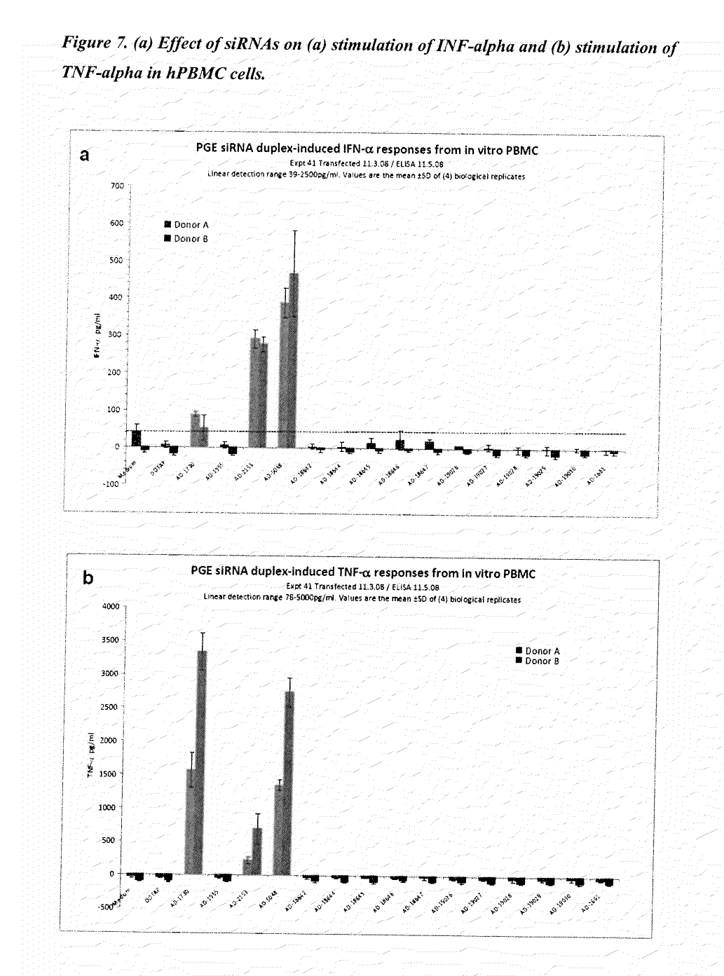

The siRNA conjugates listed in the following table were synthesized as described in the previous examples and evaluated for their immunostimulatory potential in hPBMCs. Therefore, human PBMCs were collected from tow different healthy donors and TNF-alpha and INF-alpha were measured by ELISA 48 hours after transfection of the siRNAs. As controls, the transfection reagent (DOTAP) as well as some siRNAs with known immunostimulatory potential (AD-1730, AD-1955, AD-2153, AD-5048) were used. FIGS. 7a and b show the effect of the siRNAs on stimulation of TNF-alpha and INF-alpha, respectively.

TABLE 12
Sequence information of siRNA conjugates subjected to hPBMC assay.

| Duplex | Strand | | |
|---|---|---|---|
| ID | ID | Strand | Sequence (5′ to 3′) |
| AD-18642 | 16865 | S | GccuGGAGuuuAuucGGAAdTsdT (SEQ ID NO: 38) |
| | 32596 | AS | uUCCGAAuAAACUCcAGGCdTsdT (SEQ ID NO: 35) |
| AD-18644 | 30925 | S | GccuGGAGuuuAuucGGAAQ51sds(CTTACGCTGAGTACTTC |
| | | | GAT) (SEQ ID NOs: 34, 1) |
| | 32596 | AS | uUCCGAAuAAACUCcAGGCdTsdT (SEQ ID NO: 35) |
| AD-18645 | 32814 | S | GccuGGAGuuuAuucGGAAQ51sds(CTTACGCTGAGTACTTC |
| | | | GAT) (SEQ ID NOs: 34, 46) |
| | 32596 | AS | uUCCGAAuAAACUCcAGGCdTsdT (SEQ ID NO: 35) |
| AD-18646 | 32815 | S | GGAUfCfAUfCfUfCfAAGUfCfUfUfACfQ51sds(CTTACGCTGAG |
| | | | TACTTCGAT) (SEQ ID NOs: 49, 1) |
| | 4724 | AS | GUfAAGACfUfUfGAGAUfGAUfCfCfdTsdT (SEQ ID NO: 50) |
| AD-18647 | 32816 | S | GGAUfCfAUfCfUfCfAAGUfCfUfUfACfQ51sds(CTTACGCTGAG |
| | | | TACTTCGAT) (SEQ ID NOs: 49, 46) |
| | 4724 | AS | GUfAAGACfUfUfGAGAUfGAUfCfCfdTsdT (SEQ ID NO: 50) |
| AD-1661 | 4723 | S | GGAUfCfAUfCfUfCfAAGUfCfUfUfACfdTsdT (SEQ ID NO: 51) |
| | 4724 | AS | GUfAAGACfUfUfGAGAUfGAUfCfCfdTsdT (SEQ ID NO: 50) |
| AD-19030 | 18241 | S | GfcCfuGfgAfgUfuUfaUfuCfgGfaAfdTsdT (SEQ ID NO: 45) |
| | 32596 | AS | uUCCGAAuAAACUCcAGGCdTsdT (SEQ ID NO: 35) |
| AD-19026 | 33574 | S | GfcCfuGfgAfgUfuUfaUfuCfgGfaAfdTsdTsQ50sds(CTTACGCT |
| | | | GAGTACTTCGAT) (SEQ ID NOs: 45, 46) |
| | 32596 | AS | uUCCGAAuAAACUCcAGGCdTsdT (SEQ ID NO: 35) |
| AD-19027 | 33575 | S | GfgAfuCfaUfcUfcAfaGfuCfuUfaCfdTsdTsQ50sds(CTTACGCTG |
| | | | AGTACTTCGAT) (SEQ ID NOs: 47, 46) |
| | 4724 | AS | GUfAAGACfUfUfGAGAUfGAUfCfCfdTsdT (SEQ ID NO: 50) |
| AD-19028 | 33591 | S | GfcCfuGfgAfgUfuUfaUfuCfgGfaAfdTsdIsQ50ds(GCCCAAGCT |
| | | | GGCATCCGTCA) (SEQ ID NOs: 45, 48) |
| | 32596 | AS | uUCCGAAuAAACUCcAGGCdTsdT (SEQ ID NO: 35) |
| AD-19029 | 33592 | S | GfgAfuCfaUfcUfcAfaGfuCfuUfaCfdTsdTsQ50ds(GCCCAAGCT |
| | | | GGCATCCGTCA) (SEQ ID NOs: 47, 48) |
| | 4724 | AS | GUfAAGACfUfUfGAGAUfGAUfCfCfdTsdT (SEQ ID NO: 50) |

s: phosphorothioate linkage;
lower case: 2′-O-methyl;
upper case: RNA;
Nf: 2′-fluoro nucleoside;
C: 5-methylcytidine;
Q50: (CH2)12 linker;
Q51: (CH2)6—S—S—(CH2)6 linker;
L10: cholesterol.

### Example 13

### In Vitro Silencing Activity of Modified siRNAs

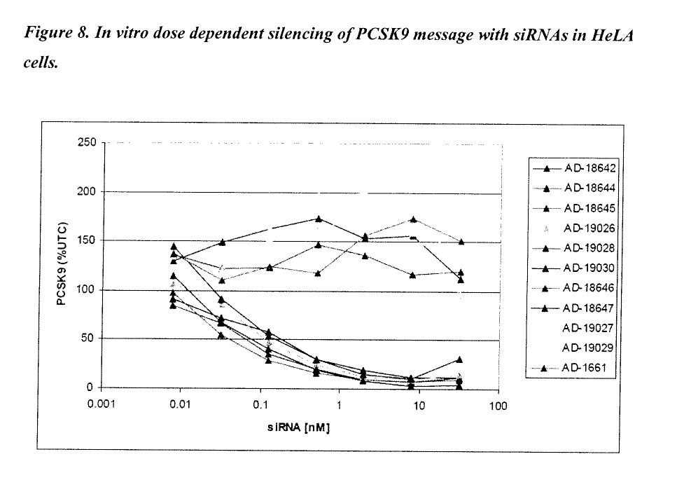

The siRNAs listed in the following table were evaluated for their ability to silence PCSK9 expression in HeLa cells after transfection with Lipofectamine-2000. The sequence information of the siRNA conjugates is listed in the following table and the results of the in vitro study are shown in FIG. 8.

TABLE 13
Sequence information of siltATA conjugates
evaluated for silencing activity in vitro.

| Duplex | Strand | | |
|---|---|---|---|
| ID | ID | Strand | Sequence (5′ to 3′) |
| AD-18642 | 16865 | S | GccuGGAGuuuAuucGGAAdTsdT (SEQ ID NO: 38) |
| | 32596 | AS | uUCCGAAuAAACUCcAGGCdTsdT (SEQ ID NO: 35) |
| AD-18644 | 30925 | S | GccuGGAGuuuAuucGGAAQ51sds(CTTACGCTGAGTACTTC |
| | | | GAT) (SEQ ID NOs: 34, 1) |
| | 32596 | AS | uUCCGAAuAAACUCcAGGCdTsdT (SEQ ID NO: 35) |
| AD-18645 | 32814 | S | GccuGGAGuuuAuucGGAAQ51sds(CTTACGCTGAGTACTTC |
| | | | GAT) (SEQ ID NOs: 34, 46) |
| | 32596 | AS | uUCCGAAuAAACUCcAGGCdTsdT (SEQ ID NO: 35) |
| AD-18646 | 32815 | S | GGAUfCfAUfCfUfCfAAGUfCfUfUfACfQ51sds(CTTACGCTGAG |
| | | | TACTTCGAT) (SEQ ID NOs: 49, 1) |
| | 4724 | AS | GUfAAGACfUfUfGAGAUfGAUfCfCfdTsdT (SEQ ID NO: 50) |
| AD-18647 | 32816 | S | GGAUfCfAUfCfUfCfAAGUfCfUfUfACfQ51sds(CTTACGCTGAG |
| | | | TACTTCGAT) (SEQ ID NOs: 49, 46) |
| | 4724 | AS | GUfAAGACfUfUfGAGAUfGAUfCfCfdTsdT (SEQ ID NO: 50) |
| AD-1661 | 4723 | S | GGAUfCfAUfCfUfCfAAGUfCfUfUfACfdTsdT (SEQ ID NO: 51) |
| | 4724 | AS | GUfAAGACfUfUfGAGAUfGAUfCfCfdTsdT (SEQ ID NO: 50) |
| AD-19030 | 18241 | S | GfcCfuGfgAfgUfuUfaUfuCfgGfaAfdTsdT (SEQ ID NO: 45) |
| | 32596 | AS | uUCCGAAuAAACUCcAGGCdTsdT (SEQ ID NO: 35) |
| AD-19026 | 33574 | S | GfcCfuGfgAfgUfuUfaUfuCfgGfaAfdTsdTsQ50sds(CTTACGCT |
| | | | GAGTACTTCGAT) (SEQ ID NOs: 45, 46) |
| | 32596 | AS | uUCCGAAuAAACUCcAGGCdTsdT (SEQ ID NO: 35) |
| AD-19027 | 33575 | S | GfgAfuCfaUfcUfcAfaGfuCfuUfaCfdTsdTsQ50sds(CTTACGCTG |
| | | | AGTACTTCGAT) (SEQ ID NOs: 47, 46) |
| | 4724 | AS | GUfAAGACfUfUfGAGAUfGAUfCfCfdTsdT (SEQ ID NO: 50) |
| AD-19028 | 33591 | S | GfcCfuGfgAfgUfuUfaUfuCfgGfaAfdTsdTsQ50ds(GCCCAAGCT |
| | | | GGCATCCGTCA) (SEQ ID NOs: 45, 48) |
| | 32596 | AS | uUCCGAAuAAACUCcAGGCdTsdT (SEQ ID NO: 35) |
| AD-19029 | 33592 | S | GfgAfuCfaUfcUfcAfaGfuCfuUfaCfdTsdTsQ50ds(GCCCAAGCT |
| | | | GGCATCCGTCA) (SEQ ID NOs: 47, 48) |
| | 4724 | AS | GUfAAGACfUfUfGAGAUfGAUfCfCfdTsdT (SEQ ID NO: 50) |

s: phosphorothioate linkage;
lower case: 2′-O-methyl;
upper case: RNA;
Nf: 2′-fluoro nucleoside;
C: 5-methylcytidine;
Q50: (CH2)12 linker;
Q51: (CH2)6—S—S—(CH2)6 linker;
L10: cholesterol.

1. An iRNA agent that comprises at least one carrier oligomer, wherein said carrier oligomer is selected from the group consisting of a single-stranded phosphorothioate oligonucleotide, a non-nucleosidic phosphorothioate oligomer, a poly(G) oligonucleotide, a poly(I) oligonucleotide, and an oligonucleotide that can form a quadruplex structure.

2. An iRNA agent of claim 1, wherein said carrier oligomer is a phosphorothioate oligonucleotide.

3. An iRNA agent of claim 2, wherein at least 10%, at least 20%, at least 30%, at least 40%, at least 50%, at least 60%, at least 70%, at least 80%, at least 90% or all backbone linkages are phosphorothioate in the said phosphorothioate oligonucleotide.

4. An iRNA agent of claim 2, wherein said oligonucleotide has the sequence dCsdTsdTdsAsdCsdGsdCsdTsdGsdAsdGsdTsdAsdCsdTsdTsdCsdGsdAsdT (SEQ ID NO: 1).

5. An iRNA agent of claim 1, wherein said carrier oligomer is a non-nucleosidic phosphorothioate oligomer.

6. An iRNA agent of claim 5, wherein said non-nucleosidic phosphorothioate oligomer is chosen from: 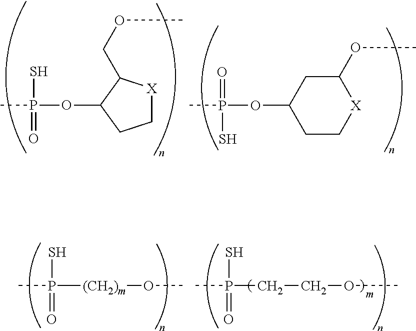
wherein:
x is independently for each occurrence CH₂O, O, NH or S;
n is independently for each occurrence 10-30; and
m is independently for each occurrence 1-2.

7. An iRNA agent of claim 1, wherein said carrier oligomer is a poly(G) oligonucleotide.

8. An iRNA agent of claim 7, wherein said poly(G) oligonucleotide is 3-14 nucleotides in length.

9. An iRNA agent of claim 8, wherein said poly(G) oligonucleotide is 5-10 nucleotides in length.

10. An iRNA agent of claim 9, wherein said poly(G) oligonucleotide is d(G)₅.

11. An iRNA agent of claim 9, wherein said poly(G) oligonucleotide is d(G)₁₀.

12. An iRNA agent of claim 1, wherein said carrier oligomer is an oligonucleotide that can form a quadruplex structure.

13. An iRNA agent of claim 12, wherein said oligonucleotide has the sequence d(GₚN_{q}GₚN_{q}GₚN_{q}Gₚ) (SEQ ID NO: 3);
wherein:
p is independently for each occurrence 3-10;
q is independently for each occurrence 1-7; and
N is independently for each occurrence an optionally modified natural or non-natural nucleobase.

14. An iRNA agent of claim 1, wherein said iRNA agent is linked to said carrier oligomer through a phosphodiester, phosphorothioate, phosphorodithioate, alkylphosphonate, amide, ester, disulfide, thioether, oxime or a hydrazone linkage.

15. An iRNA agent of claim 14, wherein said linkage is a phosphorothioate.

16. An iRNA agent of claim 1, wherein said iRNA agent is linked to said carrier oligomer through a spacer.

17. An iRNA agent of claim 16, wherein said spacer comprises a cleavable linking group.

18. An iRNA agent of claim 17, wherein said cleavable linking group is selected from the group consisting of a phosphodiester, a disulfide, an amide, an ester and a peptide linkage.

19. An iRNA agent of claim 18, wherein said cleavable linking group is a disulfide.

20. An iRNA agent of claim 16, wherein the spacer is —P(Z¹)(Z²)—O—(CH₂)ₙ¹—S—S—(CH₂)ₙ²—O—P(Z³)(Z⁴)—; wherein Z¹ and Z³ are independently O or S; Z² and Z⁴ are independently O, S, alkyl, or aminoalkyl; and n¹ and n² are each independently 1-10.

21. An iRNA agent of claim 20, wherein both n¹ and n² are 6.

22. An iRNA agent of claim 21, wherein Z¹ and Z³ are O; and Z² and Z⁴ are S.
